(19) United States
(12) Patent Application Publication
(10) Pub. No.: US 2009/0222937 A1
(43) Pub. Date: Sep. 3, 2009

(54) MEGANUCLEASE VARIANTS CLEAVING A DNA TARGET SEQUENCE FROM A XERODERMA PIGMENTOSUM GENE AND USES THEREOF

(75) Inventors: Sylvain Arnould, Saint-Denis (FR); Christophe Perez-Michaut, Paris (FR); Julianne Smith, Le Plessis Robinson (FR)

Correspondence Address: OBLON, SPIVAK, MCCLELLAND MAIER & NEUSTADT, P.C., 1940 DUKE STREET, ALEXANDRIA, VA, 22314, US

(73) Assignee: CELLECTIS, ROMAINVILLE (FR)

(21) Appl. No.: 12/279,245

(22) PCT Filed: Feb. 13, 2007
(86) PCT No.: PCT/IB2007/000924

§ 371 Date: Oct. 9, 2008

(30) Foreign Application Priority Data
Feb. 13, 2006 (IB) PCT/IB2006/000589

(51) Int. Cl. A01K 67/027; C12N 9/16; C07H 21/00; C12N 15/63; C12N 5/00; A01H 5/00

(52) U.S. Cl. 800/13; 435/196; 536/23.2; 435/320.1; 435/325; 800/298

(57) ABSTRACT

An I-CreI variant which has at least two substitutions, one in each of the two functional subdomains of the LAGLIDADG (SEQ ID NO: 229) core domain situated from positions 26 to 40 and 44 to 77 of I-CreI, said variant being able to cleave a DNA target sequence from a xeroderma pigmentosum gene. Use of said variant and derived products for the prevention and the treatment of Xeroderma pigmentosum.

The invention relates to a meganuclease variant cleaving a DNA target sequence from a xeroderma pigmentosum gene (XP gene), to a vector encoding said variant, to a cell, an animal or a plant modified by said vector and to the use of said meganuclease variant and derived products for genome therapy, in vivo and ex vivo (gene cell therapy), and genome engineering.

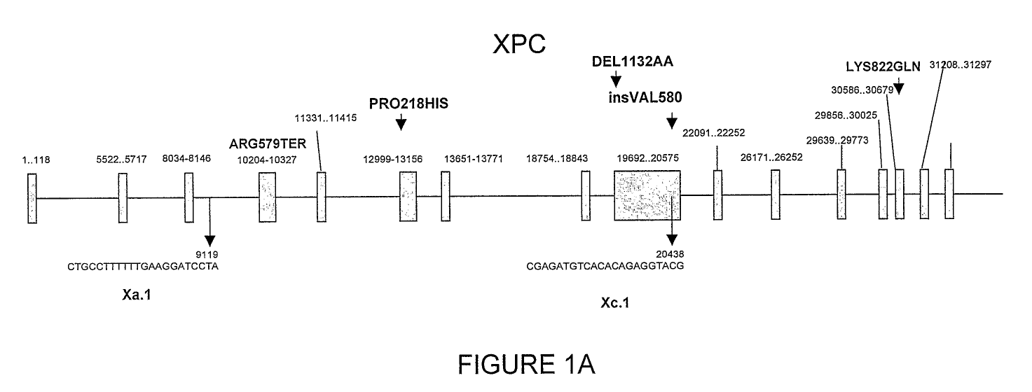

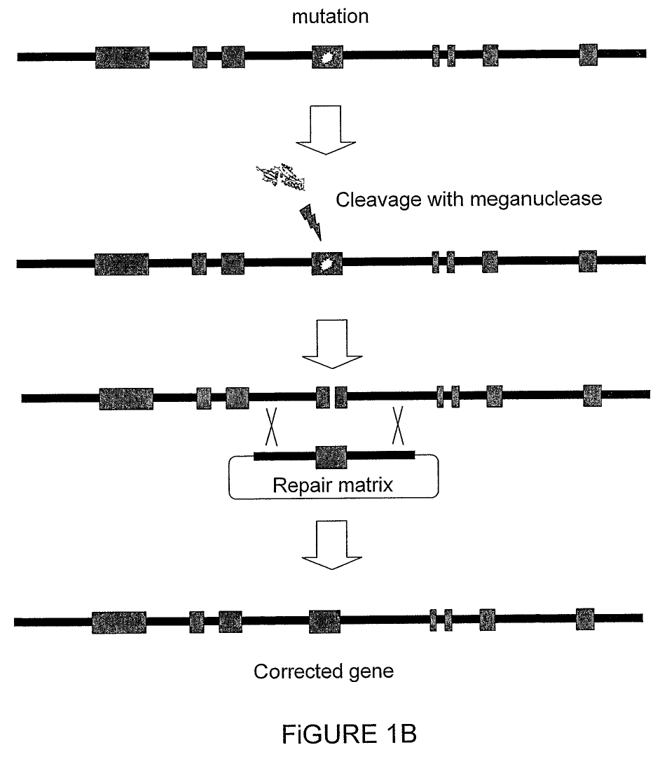

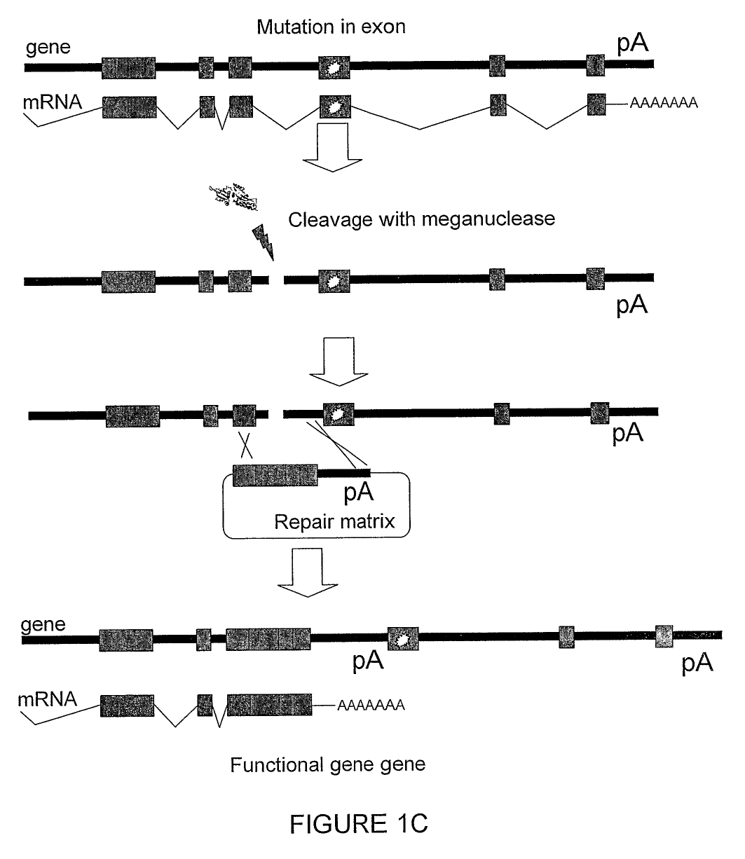

Xeroderma pigmentosum (XP) is a rare autosomal recessive genetic disease characterized by a hypersensitivity to exposure to ultraviolet A (UV) rays, a high predisposition for developing skin cancers on sunlight exposed areas, and in some cases neurological disorders (Hengge, U. R. and W. Bardenheuer, Am. J. Med. Genet. C. Semin. Med. Genet., 2004, 131: 93-100; Magnaldo, T. and A. Sarasin, Cells Tissues Organs, 2004, 177: 189-198; Cleaver, J. E., Nat. Rev. Cancer, 2005, 5: 564-573; Hengge U. R., Clin. Dermatol., 2005, 23: 107-114). Cells of XP patients present a reduced capacity to eliminate UV induced DNA lesions (Cordonnier, A. M. and R. P. Fuchs, Mutat. Res., 1999, 435, 111-119). Such abnormality results from a defect in the Nucleotide Excision Repair (NER) process, a versatile mechanism conserved among eukaryotes and implicated in the correction of the damaged DNA by excision of the damaged nucleotides and re-synthesis. Defect in this process leads to a persistence of UV damage in the DNA, resulting in mutagenesis and tumour development in the UV exposed skin area. The three major types of skin cancers, squamous cell carcinomas, basal cell carcinomas and malignant melanomas already appear in childhood. XP Patients were assigned to 7 complementation groups (XP-A to XP-G) by cell fusion experiments, and each complementation group turned out to result from mutations in a distinct NER gene. The human genes, and the encoded proteins were often named after the complementation group. For example, the XPC gene (FIG. 1A), mutated in the XP-C complementation group, codes for a DNA damage binding protein.

Until now the only treatment available to XP patients is either full protection against sun exposure (as well as against certain common lamps producing long-wavelength UV) or repeated surgery to remove appearing skin cancers. Several attempts of autologous graft have been made to replace such cancerous area with skin from unexposed parts of the patient's body. However, since the grafted cells are also sun-sensitive the benefits for the patients are at best, limited to a few years, and the majority of patients die before reaching adulthood because of metastases. Skin engraftment can be made locally, but with the general limitations of grafts, in term of immunological tolerance.

Thus gene and cell therapy represent a huge hope for this kind of disease. Since cells from the skin lineage can be easily manipulated in vitro, a possibility would be to manipulate patient cells and correct their genetic defect, before grafting them back at the site of the tumour. Compared to other XP complementation groups, XP-C seems to be the best candidate for corrective gene transfer. In Europe and North Africa, XP-C is involved in more than half of the XP patients and although XPC expression is ubiquitous, XP-C patients remain free of neurological problems observed in other XP groups. Preliminary studies aimed at tissue therapy of XP patients have shown that in vitro retroviral transduction of XP fibroblasts from various complementation groups (XP-A, XP-B, XP-C, XP-D) and of XP-C primary keratinocytes with the XP cloned genes result in the recovery of full DNA repair capacity (Arnaudeau-Begard et al., Hum. Gene Ther., 2003, 14, 983-996; Armelini et al., Cancer Gene Ther., 2005, 12, 389-396). Furthermore, cells from the skin lineage can be easily manipulated, and then used to reconstruct functional skin (Amaudeau-Begard et al., Hum. Gene Ther., 2003, 14, 983-996; Armelini et al., Cancer Gene Ther., 2005, 12, 389-396). Thus, a rationale and promising alternative for long term tissular therapy would then consist in an ex vivo gene correction of the XP-C locus in keratinocytes before grafting back a reconstructed skin to the patient.

Homologous recombination is the best way to precisely engineer a given locus. Homologous gene targeting strategies have been used to knock out endogenous genes (Capecchi, M. R., Science, 1989, 244: 1288-1292; Smithies O., Nat. Med., 2001, 7: 1083-1086) or knock-in exogenous sequences in the chromosome.

It can as well be used for gene correction, and in principle, for the correction of mutations linked with monogenic diseases, such as XP. However, this application is in fact difficult, due to the low efficiency of the process (10⁻⁶ to 10⁻⁹ of transfected cells). In the last decade, several methods have been developed to enhance this yield. For example, chimeraplasty (De Semir et al. J. Gene Med., 2003, 5: 625-639) and Small Fragment Homologous Replacement (Goncz et al., Gene Ther, 2001, 8: 961-965; Bruscia et al., Gene Ther., 2002, 9: 683-685; Sangiuolo et al., BMC Med. Genet., 2002, 3: 8-; De Semir and Aran, Oligonucleotides, 2003, 13: 261-269; U.S. Pat. No. 6,010,908) have both been used to try to correct CFTR mutations with various levels of success.

Another strategy to enhance the efficiency of recombination is to deliver a DNA double-strand break in the targeted locus, using meganucleases. Meganucleases are by definition sequence-specific endonucleases recognizing large sequences (12 to 45 bp). They can cleave unique sites in living cells, thereby enhancing gene targeting by 1000-fold or more in the vicinity of the cleavage site (Puchta et al., Nucleic Acids Res., 1993, 21: 5034-5040; Rouet et al., Mol. Cell. Biol., 1994, 14, 8096-8106; Choulika et al., Mol. Cell. Biol., 1995, 15, 1968-1973; Puchta et al., Proc. Natl. Acad. Sci. USA, 1996, 93, 5055-5060; Sargent et al., Mol. Cell. Biol., 1997, 17, 267-277; Donoho et al., Mol. Cell. Biol, 1998, 18, 4070-4078; Elliott et al., Mol. Cell. Biol., 1998, 18, 93-101; Cohen-Tannoudji et al., Mol. Cell. Biol., 1998, 18, 1444-1448). Recently, I-SceI was used to stimulate targeted recombination in mouse hepatocytes in vivo. Recombination could be observed in up to 1% of hepatocytes (Gouble et al., J. Gene Med., 2006, 8, 616-622).

However, the use of this technology is limited by the repertoire of natural meganucleases. For example, there is no cleavage site for a known natural meganuclease in human XP genes. Therefore, the making of meganucleases with tailored specificities is under intense investigation, and several laboratories have tried to alter the specificity of natural meganucleases or to make artificial endonuclease.

Recently, fusion of Cys2-His2 type Zinc-Finger Proteins (ZFP) with the catalytic domain of the Type IIS FokI endonuclease were used to make functional sequence-specific artificial endonucleases (Smith et al., Nucleic Acids Res., 1999, 27: 674-681; Bibikova et al., Science, 2003, 300: 764; Porteus M. H. and D. Baltimore, Science, 2003, 300: 763). The binding specificity of ZFPs is relatively easy to manipulate, and a repertoire of novel artificial ZFPs, able to bind many (g/a)nn(g/a)nn(g/a)nn sequences is now available (Pabo et al., Annu. Rev. Biochem., 2001, 70, 313-340; Segal, D. J. and C. F. Barbas, Curr. Opin. Biotechnol., 2001, 12, 632-637; Isalan et al., Nat. Biotechnol., 2001, 19, 656-660). This last strategy allowed recently for the engineering of the IL2RG gene in vitro (Urnov et al., Nature, 2005, 435, 646-651). Nevertheless, preserving a very narrow specificity is one of the major issues for genome engineering applications, and presently it is unclear whether ZFPs would fulfill the very strict requirements for therapeutic applications.

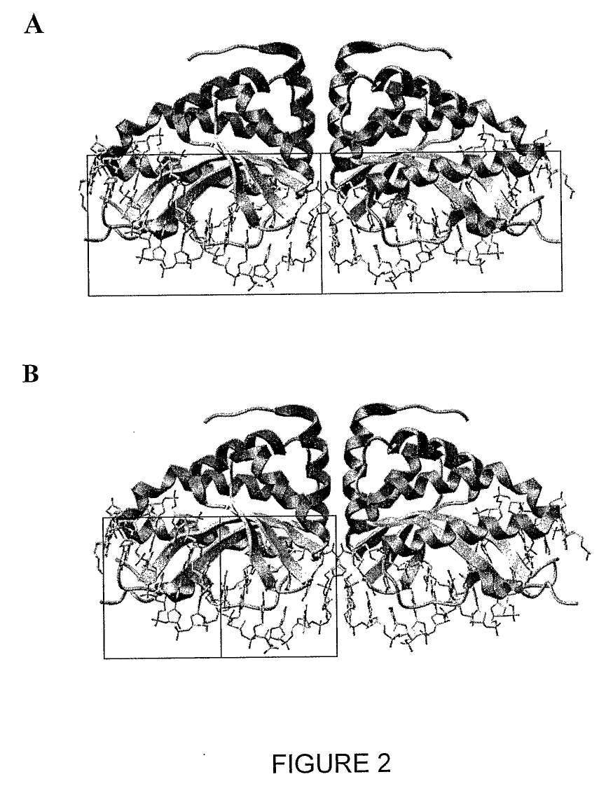

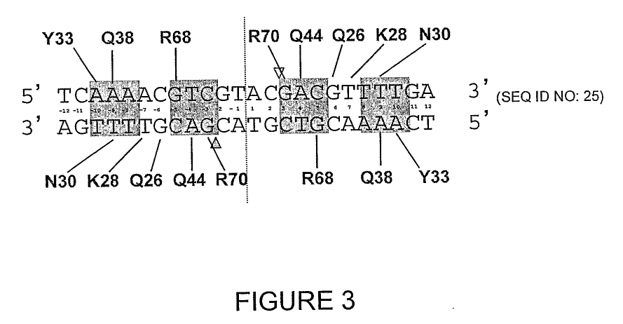

Homing Endonucleases (HEs) are a widespread family of natural meganucleases including hundreds of proteins (Chevalier, B. S, and B. L. Stoddard, Nucleic Acids Res., 2001, 29, 3757-3774). These proteins are encoded by mobile genetic elements which propagate by a process called “homing”: the endonuclease cleaves a cognate allele from which the mobile element is absent, thereby stimulating a homologous recombination event that duplicates the mobile DNA into the recipient locus (Kostriken et al., Cell; 1983, 35, 167-174; Jacquier, A. and B. Dujon, Cell, 1985, 41, 383-394). Given their natural function and their exceptional cleavage properties in terms of efficacy and specificity, HEs provide ideal scaffolds to derive novel endonucleases for genome engineering. Data have been accumulated over the last decade, characterizating the LAGLIDADG family, the largest of the four HE families (Chevalier and Stoddard, precited). LAGLIDADG refers to the only sequence actually conserved throughout the family and is found in one or (more often) two copies in the protein. Proteins with a single motif, such as I-CreI, form homodimers and cleave palindromic or pseudo-palindromic DNA sequences, whereas the larger, double motif proteins, such as I-SceI are monomers and cleave non-palindromic targets. Seven different LAGLIDADG proteins have been crystallized, and they exhibit a very striking conservation of the core structure, that contrasts with the lack of similarity at the primary sequence level (Jurica et al., Mol. Cell., 1998, 2, 469-476; Chevalier et al., Nat. Struct. Biol., 2001, 8, 312-316; Chevalier et al., J. Mol. Biol., 2003, 329, 253-269; Moure et al., J. Mol. Biol, 2003, 334, 685-695; Moure et al., Nat. Struct. Biol., 2002, 9, 764-770; Ichiyanagi et al., J. Mol. Biol., 2000, 300, 889-901; Duan et al., Cell, 1997, 89, 555-564; Bolduc et al., Genes Dev., 2003, 17, 2875-2888; Silva et al., J. Mol. Biol., 1999, 286, 1123-1136). In this core structure, two characteristic αββαββα folds, also called LAGLIDADG Homing Endonuclease Core Domains, contributed by two monomers, or by two domains in double LAGLIDAG proteins, are facing each other with a two-fold symmetry. DNA binding depends on the four β strands from each domain, folded into an antiparallel β-sheet, and forming a saddle on the DNA helix major groove (FIG. 2). Analysis of I-CreI structure bound to its natural target shows that in each monomer, eight residues (Y33, Q38, N30, K28, Q26, Q44, R68 and R70) establish direct interactions with seven bases at positions ±3, 4, 5, 6, 7, 9 and 10 (Jurica et al., 1998, precited; FIG. 3). In addition, some residues establish water-mediated contact with several bases; for example S40 and N30 with the base pair at position +8 and −8 (Chevalier et al., 2003, precited). The catalytic core is central, with a contribution of both symmetric monomers/domains. In addition to this core structure, other domains can be found: for example, PI-SceI, an intein, has a protein splicing domain, and an additional DNA-binding domain (Moure et al., 2002, precited; Grindl et al., Nucleic Acids Res., 1998, 26, 1857-1862).

Two approaches have been used to derive novel endonucleases with new specificities, from Homing Endonucleases:

Protein Variants

Seligman and co-workers used a rational approach to substitute specific individual residues of the I-CreI αββαββα fold (Sussman et al., J. Mol. Biol., 2004, 342, 31-41; Seligman et al., Genetics, 1997, 147, 1653-64); substantial cleavage was observed for few I-CreI variants (Y33C, Y33H, Y33R, Y33L, Y33S, Y33T, S32K, S32R) and only for a target modified in position ±10.

In a similar way, Gimble et al. modified the additional DNA binding domain of PI-SceI (J. Mol. Biol., 2003, 334, 993-1008); they obtained protein variants with altered binding specificity but no altered specificity and most of the variants maintained a lot of affinity for the wild-type target sequence.

The semi-rational approach used in theses studies permits the identification of endonucleases with altered specificity; however, it does not allow the direct production of endonucleases with predicted specificity.

Hybrid or Chimeric Single-Chain Proteins

New meganucleases could be obtained by swapping LAGLIDADG Homing Endonuclease Core Domains of different monomers (Epinat et al., Nucleic Acids Res., 2003, 31, 2952-62; Chevalier et al., Mol. Cell., 2002, 10, 895-905; Steuer et al., Chembiochem., 2004, 5, 206-13; International PCT Applications WO 03/078619 and WO 2004/031346). These single-chain chimeric meganucleases wherein the two LAGLIDADG Homing Endonuclease Core Domains from different meganucleases are linked by a spacer, are able to cleave the hybrid target corresponding to the fusion of the two half parent DNA target sequences.

The construction of chimeric and single chain artificial HEs has suggested that a combinatorial approach could be used to obtain novel meganucleases cleaving novel (non-palindromic) target sequences: different monomers or core domains could be fused in a single protein, to achieve novel specificities. These results mean that the two DNA binding domains of an I-CreI dimer behave independently; each DNA binding domain binds a different half of the DNA target site (FIG. 2A). Recently, a two steps strategy was used to tailor the specificity of a natural HEs such as I-CreI (Arnould et al., J. Mol. Biol., 2006, 355: 443-458). In a first step, residues Q44, R68 and R70 were mutagenized, and a collection of variants with altered specificity in positions ±3 to 5 (5NNN DNA target) were identified by screening. In a second step, two different variants were combined and assembled in a functional heterodimeric endonuclease able to cleave a chimeric target resulting from the fusion of a different half of each variant DNA target sequence.

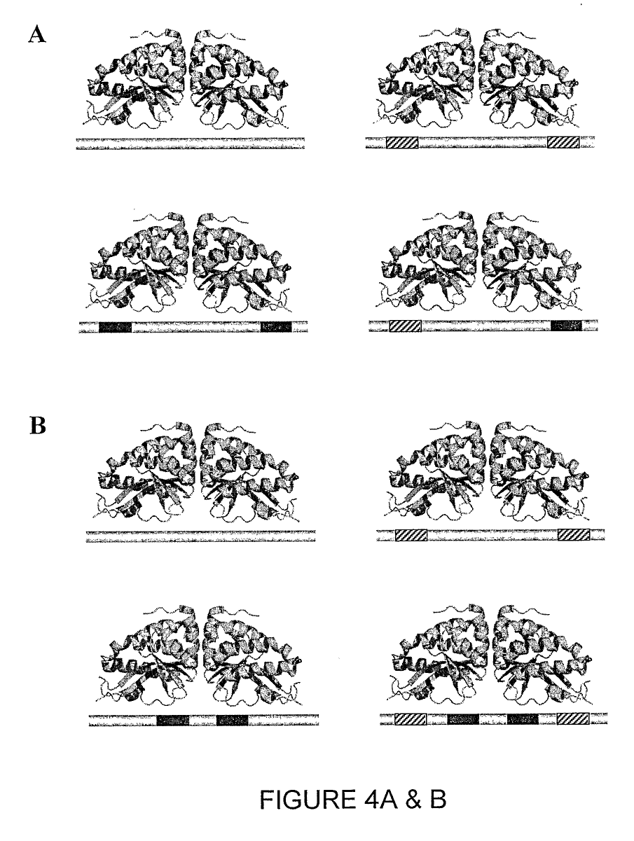

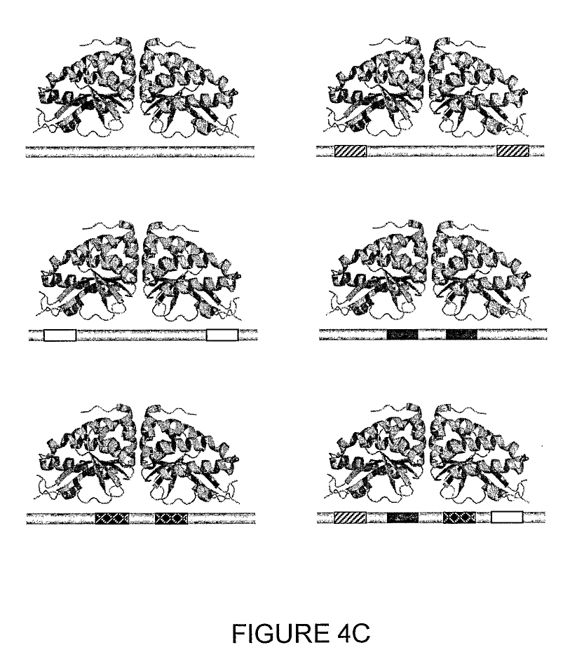

The generation of collections of novel meganucleases, and the ability to combine them by assembling two different monomers/core domains considerably enriches the number of DNA sequences that can be targeted (FIG. 4A), but does not yet saturate all potential sequences.

To reach a larger number of sequences, it would be extremely valuable to be able to identify smaller independent subdomains that could be combined (FIG. 2B).

However, a combinatorial approach is much more difficult to apply within a single monomer or domain than between monomers since the structure of the binding interface is very compact and the two different ββ hairpins which are responsible for virtually all base-specific interactions do not constitute separate subdomains, but are part of a single fold. For example, in the internal part of the DNA binding regions of I-CreI, the gtc triplet is bound by one residue from the first hairpin (Q44), and two residues from the second hairpin (R68 and R70; see FIG. 1B of Chevalier et al., 2003, precited).

A semi rational design assisted by yeast high throughput screening method allowed the Inventors, to identify and isolate thousands of I-CreI variants in positions 28, 30, 33, 38 and 40 with altered specificities in positions ±8 to 10 (10NNN DNA target). These new proteins were designed to cleave one of the 64 targets degenerate at nucleotides ±10, ±9, ±8 (10NNN DNA target) of the I-CreI original target site (FIG. 3). Furthermore, in spite of the lack of apparent modularity at the structural level, residues 28 to 40 binding to positions ±8 to 10 and residues 44 to 77 binding to positions ±3 to 5 of the I-CreI site, were revealed to form two separable functional subdomains, able to bind distinct parts of an I-CreI homing endonuclease half-site (FIG. 3 and FIG. 4B). By assembling two subdomains from different monomers or core domains within the same monomer, the Inventors have engineered functional homing endonuclease (homodimeric) variants, which are able to cleave palindromic chimeric targets (FIG. 4B) having the nucleotides in positions ±3 to 5 and ±8 to 10 of each parent monomer/core domain. Furthermore, a larger combinatorial approach is allowed by assembling four different subdomains (FIG. 4C: top right, middle left and right, bottom left) to form new heterodimeric molecules which are able to cleave non-palindromic chimeric targets (bottom right). The different subdomains can be modified separately and combine in one meganuclease variant (heterodimer or single-chain molecule) which is able to cleave a target from a gene of interest. The engineered variant can be used for gene correction via double-strand break induced recombination (FIGS. 1B and 1C).

The capacity to combine four sub-domains considerably increases the number of DNA sequences that can be targeted (FIG. 4C). However, it is still difficult to fully appreciate the range of sequences that can be reached with this combinatorial approach. One of the most elusive factors is the impact of the four central nucleotides of the I-CreI target site (gtac in the palindromic I-CreI site C1221, FIG. 3). Even though the base-pairs ±1 and ±2 do not display any contact with the protein, it has been shown that these positions are not devoid of content information (Chevalier et al., J. Mol. Biol., 2003, 329, 253-269), especially for the base-pair ±1 and could be a source of additional substrate specificity (Argast et al., J. Mol. Biol., 1998, 280, 345-353; Jurica et al., Mol. Cell., 1998, 2, 469-476; Chevalier, B. S, and B. L. Stoddard, Nucleic Acids Res., 2001, 29, 3757-3774). In vitro selection of cleavable I-CreI target (Argast et al., precited) randomly mutagenized, revealed the importance of these four base-pairs on protein binding and cleavage activity. It has been suggested that the network of ordered water molecules found in the active site was important for positioning the DNA target (Chevalier et al., Biochemistry, 2004, 43, 14015-14026). In addition, the extensive conformational changes that appear in this region upon I-CreI binding suggest that the four central nucleotides could contribute to the substrate specificity, possibly by sequence dependent conformational preferences (Chevalier et al., 2003, precited).

Thus, it was not clear if mutants identified on 10NNN and 5NNN DNA targets as homodimers cleaving a palindromic sequence with the four central nucleotides being gtac, would allow the design of new endonucleases that would cleave targets containing changes in the four central nucleotides.

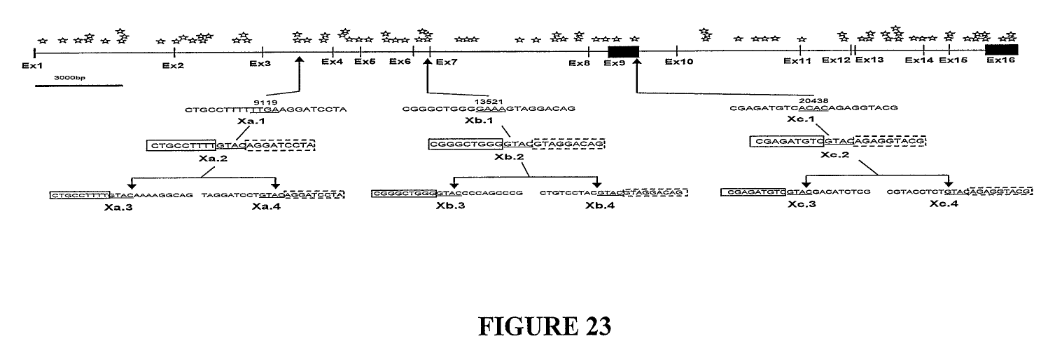

The Inventors have identified hundreds of DNA targets in the XP genes that could be cleaved by I-CreI variants. The combinatorial strategy described in FIG. 4 was used to extensively redesign the DNA binding domain of the I-CreI protein and thereby engineer novel meganucleases with fully engineered specificity, to cleave two DNA targets from the XPC gene (Xa.1 and Xc.1) which differ from the I-CreI C1221 22 bp palindromic site by 17 nucleotides including the four central nucleotides in positions ±1 to 2 (Xa.1, FIGS. 3, 9 and 23) or 11 nucleotides including two (positions −1 and −2) of the four central nucleotides (Xc.1, FIG. 23).

Even though the combined variants were initially identified towards nucleotides 10NNN and 5NNN respectively, and a strong impact of the four central nucleotides of the target on the activity of the engineered meganuclease was observed, functional meganucleases with a profound change in specificity regarding the other base-pairs of the target were selected. Furthermore, the activity of the engineered protein could be significantly improved by two successive rounds of random mutagenesis and screening, to compare with the activity of the I-CreI protein. Finally, the extensive redesign of the DNA binding domain is not made at the expense of the level of specificity, the novel endonucleases keeping a very narrow numbers of cleavable cognate targets.

These I-CreI variants which are able to cleave a DNA target sequence from a XP gene can be used for repairing the mutations associated with Xeroderma pigmentosum. Other potential applications include genome engineering at the XP genes loci.

The invention relates to an I-CreI variant which has at least two substitutions, one in each of the two functional subdomains of the LAGLIDADG core domain situated from positions 26 to 40 and 44 to 77 of I-CreI, and is able to cleave a DNA target sequence from a xeroderma pigmentosum (XP) gene.

The cleavage activity of the variant according to the invention may be measured by any well-known, in vitro or in vivo cleavage assay, such as those described in the International PCT Application WO 2004/067736 or in Arnould et al., J. Mol. Biol., 2006, 355: 443-458. For example, the cleavage activity of the variant of the invention may be measured by a direct repeat recombination assay, in yeast or mammalian cells, using a reporter vector. The reporter vector comprises two truncated, non-functional copies of a reporter gene (direct repeats) and the genomic DNA target sequence within the intervening sequence, cloned in a yeast or a mammalian expression vector. Expression of the variant results in a functional endonuclease which is able to cleave the genomic DNA target sequence. This cleavage induces homologous recombination between the direct repeats, resulting in a functional reporter gene, whose expression can be monitored by appropriate assay.

### DEFINITIONS

Amino acid residues in a polypeptide sequence are designated herein according to the one-letter code, in which, for example, Q means Gln or Glutamine residue, R means Arg or Arginine residue and D means Asp or Aspartic acid residue.

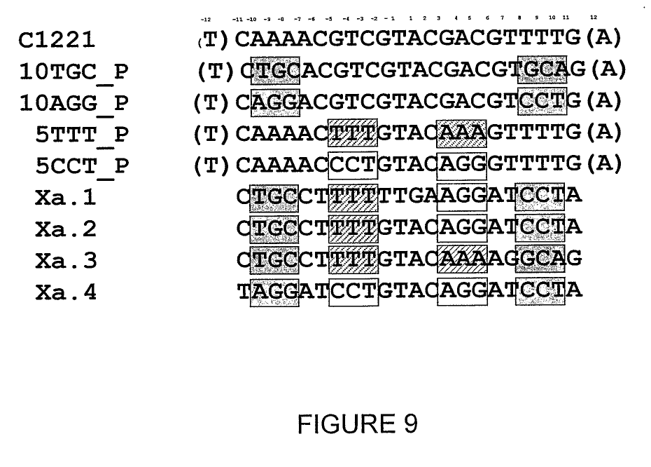

Nucleotides are designated as follows: one-letter code is used for designating the base of a nucleoside: a is adenine, t is thymine, c is cytosine, and g is guanine. For the degenerated nucleotides, r represents g or a (purine nucleotides), k represents g or t, s represents g or c, w represents a or t, m represents a or c, y represents t or c (pyrimidine nucleotides), d represents g, a or t, v represents g, a or c, b represents g, t or c, h represents a, t or c, and n represents g, a, t or c.
by “I-CreI” is intended the wild-type I-CreI having the sequence SWISSPROT P05725, corresponding to SEQ ID NO: 217 in the sequence listing, or pdb accession code 1g9y.
by “I-CreI variant” or “variant” is intended a protein obtained by replacement of at least one amino acid of I-CreI with a different amino acid.
by “functional I-CreI variant” is intended a I-CreI variant which is able to cleave a DNA target, preferably a DNA target which is not cleaved by I-CreI. For example, such variants have amino acid variation at positions contacting the DNA target sequence or interacting directly or indirectly with said DNA target.
by “I-CreI variant with novel specificity” is intended a variant having a pattern of cleaved targets different from that of the parent meganuclease. The terms “novel specificity”, “modified specificity”, “novel cleavage specificity”, “novel substrate specificity” which are equivalent and used indifferently, refer to the specificity of the variant towards the nucleotides of the DNA target sequence.
by “I-CreI site” is intended a 22 to 24 bp double-stranded DNA sequence which is cleaved by I-CreI. I-CreI sites include the wild-type (natural) non-palindromic I-CreI homing site and the derived palindromic sequences such as the sequence 5′-t₋₁₂c₋₁₁a₋₁₀a₋₉a₋₈a₋₇c₋₆g₋₅t₋₄c₋₃g₋₂t₋₁a₊₁c₊₂g₊₃a₊₄c₊₅g₊₆t₊₇t₊₈t₊₉t₊₁₀g₊₁₁a₋₁₂ (SEQ ID NO:25), also called C1221 (FIGS. 3 and 9).
by “domain” or “core domain” is intended the “LAGLIDADG Homing Endonuclease Core Domain” which is the characteristic α₁β₁β₂α₂β₃β₄α₃ fold of the homing endonucleases of the LAGLIDADG family, corresponding to a sequence of about one hundred amino acid residues. Said domain comprises four beta-strands (β₁, β₂, β₃, β₄) folded in an antiparallel beta-sheet which interacts with one half of the DNA target. This domain is able to associate with another LAGLIDADG Homing Endonuclease Core Domain which interacts with the other half of the DNA target to form a functional endonuclease able to cleave said DNA target. For example, in the case of the dimeric homing endonuclease I-CreI (163 amino acids), the LAGLIDADG Homing Endonuclease Core Domain corresponds to the residues 6 to 94.
by “subdomain” is intended the region of a LAGLIDADG Homing Endonuclease Core Domain which interacts with a distinct part of a homing endo-nuclease DNA target half-site. Two different subdomains behave independently and the mutation in one subdomain does not alter the binding and cleavage properties of the other subdomain. Therefore, two subdomains bind distinct part of a homing endonuclease DNA target half-site.
by “beta-hairpin” is intended two consecutive beta-strands of the antiparallel beta-sheet of a LAGLIDADG homing endonuclease core domain (β₁β₂ or, β₃β₄) which are connected by a loop or a turn,
by “single-chain meganuclease”, “single-chain chimeric meganuclease”, “single-chain meganuclease derivative”, “single-chain chimeric meganuclease derivative” or “single-chain derivative”, is intended a meganuclease comprising two LAGLIDADG homing endonuclease domains or core domains linked by a peptidic spacer. The single-chain meganuclease is able to cleave a chimeric DNA target sequence comprising one different half of each parent meganuclease target sequence.
by “DNA target”, “DNA target sequence”, “target sequence”, “target-site”, “target”, “site”; “site of interest”; “recognition site”, “recognition sequence”, “homing recognition site”, “homing site”, “cleavage site” is intended a 20 to 24 bp double-stranded palindromic, partially palindromic (pseudo-palindromic) or non-palindromic polynucleotide sequence that is recognized and cleaved by a LAGLIDADG homing endonuclease. These terms refer to a distinct DNA location, preferably a genomic location, at which a double stranded break (cleavage) is to be induced by the endonuclease. The DNA target is defined by the 5′ to 3′ sequence of one strand of the double-stranded polynucleotide, as indicated above for C1221. Cleavage of the DNA target occurs at the nucleotides in positions +2 and −2, respectively for the sense and the antisense strand. Unless otherwise indicated, the position at which cleavage of the DNA target by an I-Cre I meganuclease variant occurs, corresponds to the cleavage site on the sense strand of the DNA target.
by “DNA target half-site”, “half cleavage site” or half-site” is intended the portion of the DNA target which is bound by each LAGLIDADG homing endonuclease core domain.
by “chimeric DNA target” or “hybrid DNA target” is intended the fusion of a different half of two parent meganucleases target sequences. In addition at least one half of said target may comprise the combination of nucleotides which are bound by at least two separate subdomains (combined DNA target).
by “XP gene” is intended a gene of one the xeroderma pigmentosum complementation groups (XP-A, XP-B, XP-C, XP-D, XP-E, XP-f) of a mammal. For example, the human XP genes are available in the NCBI database, under the indicated accession numbers: XPA: GeneID:7507, ACCESSION NC_{—}000009, REGION: complement (97516747 . . . 97539194); XPB: GeneID:2071, ACCESSION NC_{—}000002, REGION: complement (127731096 . . . 127767982); XPC: GeneID:7508, ACCESSION NC_{—}000003, REGION: complement (14161651 . . . 14195087); XPD: GeneID:2068, ACCESSION NC_{—}000019, REGION: complement(50546686 . . . 50565669); XPE: GeneID:1642, ACCESSION NC_{—}000011, REGION: complement (60823502 . . . 60857125); XPF: GeneID:2072, ACCESSION NC_{—}000016, REGION: 13921524 . . . 13949705; XPG: GeneID:2073, ACCESSION NC_{—}000013, REGION: 102296421 . . . 102326346.
by “DNA target sequence from a XP gene” “genomic DNA target sequence”, “genomic DNA cleavage site”, “genomic DNA target” or “genomic target” is intended a 20 to 24 bp sequence of a XP gene of a mammal which is recognized and cleaved by a meganuclease variant or a single-chain chimeric meganuclease derivative.
by “vector” is intended a nucleic acid molecule capable of transporting another nucleic acid to which it has been linked.
by “homologous” is intended a sequence with enough identity to another one to lead to a homologous recombination between sequences, more particularly having at least 95% identity, preferably 97% identity and more preferably 99%.
“Identity” refers to sequence identity between two nucleic acid molecules or polypeptides. Identity can be determined by comparing a position in each sequence which may be aligned for purposes of comparison. When a position in the compared sequence is occupied by the same base, then the molecules are identical at that position. A degree of similarity or identity between nucleic acid or amino acid sequences is a function of the number of identical or matching nucleotides at positions shared by the nucleic acid sequences. Various alignment algorithms and/or programs may be used to calculate the identity between two sequences, including FASTA, or BLAST which are available as a part of the GCG sequence analysis package (University of Wisconsin, Madison, Wis.), and can be used with, e.g., default settings.
“individual” includes mammals, as well as other vertebrates (e.g., birds, fish and reptiles). The terms “mammal” and “mammalian”, as used herein, refer to any vertebrate animal, including monotremes, marsupials and placental, that suckle their young and either give birth to living young (eutharian or placental mammals) or are egg-laying (metatharian or nonplacental mammals). Examples of mammalian species include humans and other primates (e.g., monkeys, chimpanzees), rodents (e.g., rats, mice, guinea pigs) and ruminants (e.g., cows, pigs, horses).
by mutation is intended the substitution, deletion, addition of one or more nucleotides/amino acids in a polynucleotide (cDNA, gene) or a polypeptide sequence. Said mutation can affect the coding sequence of a gene or its regulatory sequence. It may also affect the structure of the genomic sequence or the structure/stability of the encoded mRNA.

According to the present invention, the positions of the mutations are indicated by reference to the I-CreI amino acid sequence SEQ ID NO: 217.

In a preferred embodiment of said variant, said substitution(s) in the subdomain situated from positions 44 to 77 of I-CreI are in positions 44, 68, 70, 75 and/or 77.

In another preferred embodiment of said variant, said substitution(s) in the subdomain situated from positions 26 to 40 of I-CreI are in positions 28, 30, 32, 33, 38 and/or 40.

In another preferred embodiment of said variant, said substitution(s) are in the subdomains situated from positions 28 to 40 and 44 to 70 of I-CreI, preferably in positions 28, 30, 32, 33, 38, 44, 68 and/or 70.

In another preferred embodiment of said variant, it comprises the substitution of the aspartic acid in position 75 by an uncharged amino acid, preferably an asparagine (D75N) or a valine (D75V).

In another preferred embodiment of said variant, it comprises one or more substitutions at additional positions contacting the DNA target sequence or interacting directly or indirectly with said DNA target. The I-CreI interacting residues are well-known in the art. The residues which are mutated may interact with the DNA backbone or with the nucleotide bases, directly or via water molecule.

In another preferred embodiment of said variant, it comprises one or more additional mutations that improve the binding and/or the cleavage properties of the variant towards the DNA target sequence of the XP gene. The additional residues which are mutated may be on the entire I-CreI sequence. These mutations may be substitutions in positions 19, 24, 42, 69, 80, 85, 87, 87, 109, 133 and 161. These mutations may affect the active site (position 19), the protein-DNA interface (for example, position 69), the hydrophobic core (for example, positions 85, 87 or 109) or the C-terminal part (for example, position 161).

In yet another preferred embodiment of said variant, said substitutions are replacement of the initial amino acids with amino acids selected from the group consisting of: A, D, E, G, H, K, N, P, Q, R, S, T, Y, C, W, L and V.

The variant according to the present invention may be an homodimer which is able to cleave a palindromic or pseudo-palindromic DNA target sequence. Alternatively, said variant is an heterodimer, resulting from the association of a first and a second monomer having different mutations in positions 26 to 40 and 44 to 77 of I-CreI, preferably in positions 28 to 40 and 44 to 70, said heterodimer being able to cleave a non-palindromic DNA target sequence from a XP gene.

The DNA target sequence which is cleaved by said variant may be in an exon or in an intron of the XP gene. Preferably, it is located, either in the vicinity of a mutation, preferably within 500 bp of the mutation, or upstream of a mutation, preferably upstream of all the mutations of said XP gene.

In another preferred embodiment of said variant, said DNA target sequence is from a human XP gene (XPA to XPG genes).

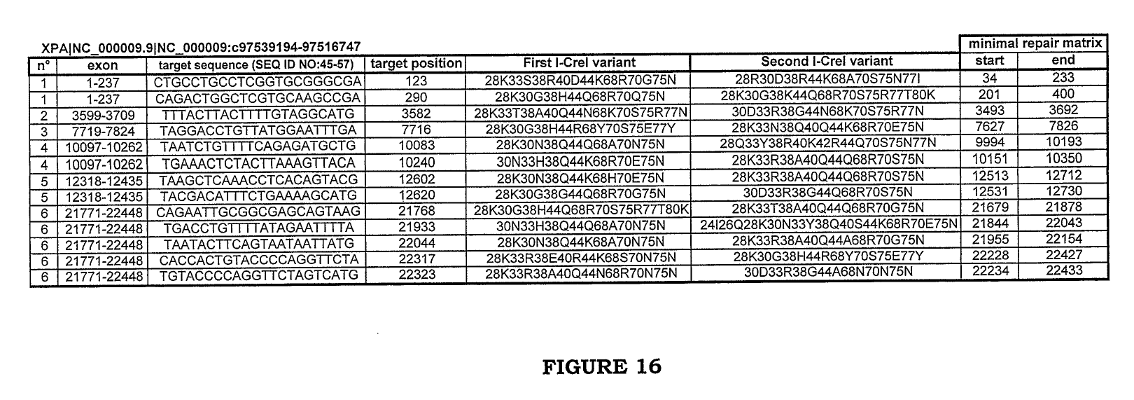

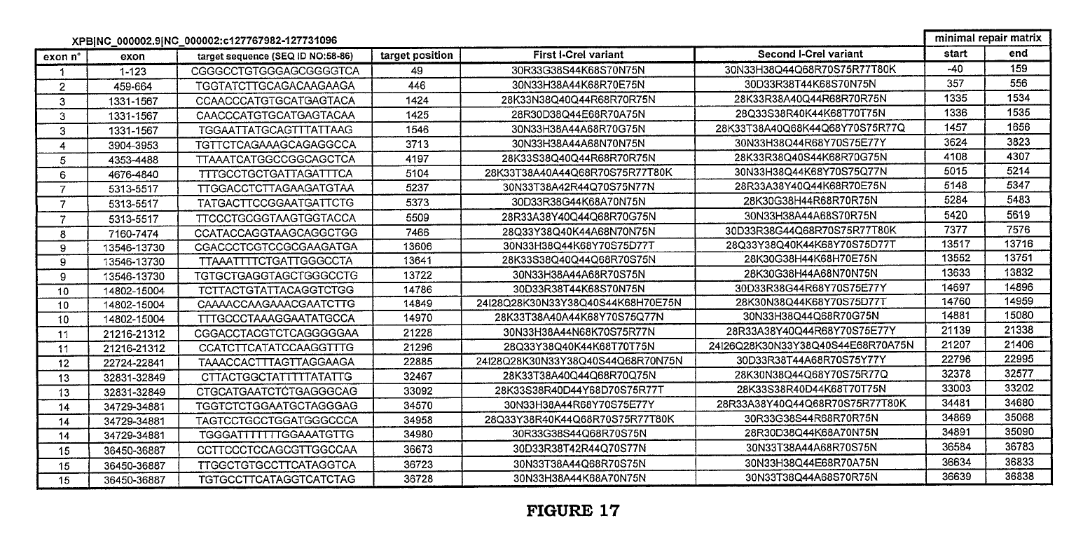

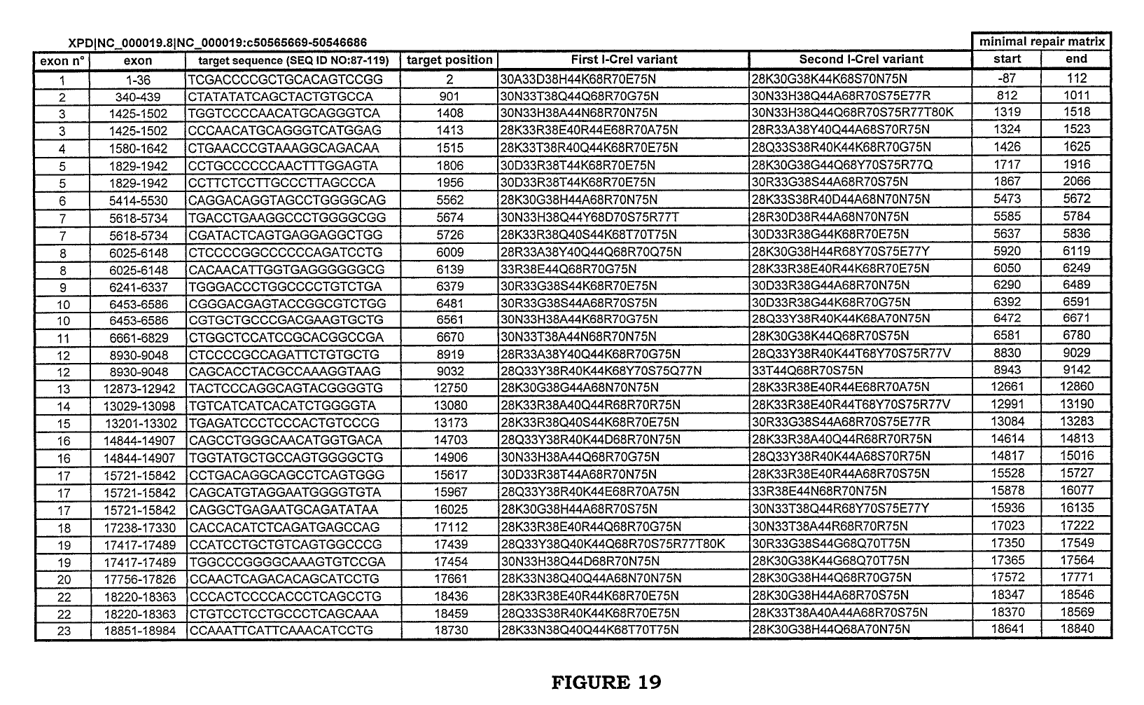

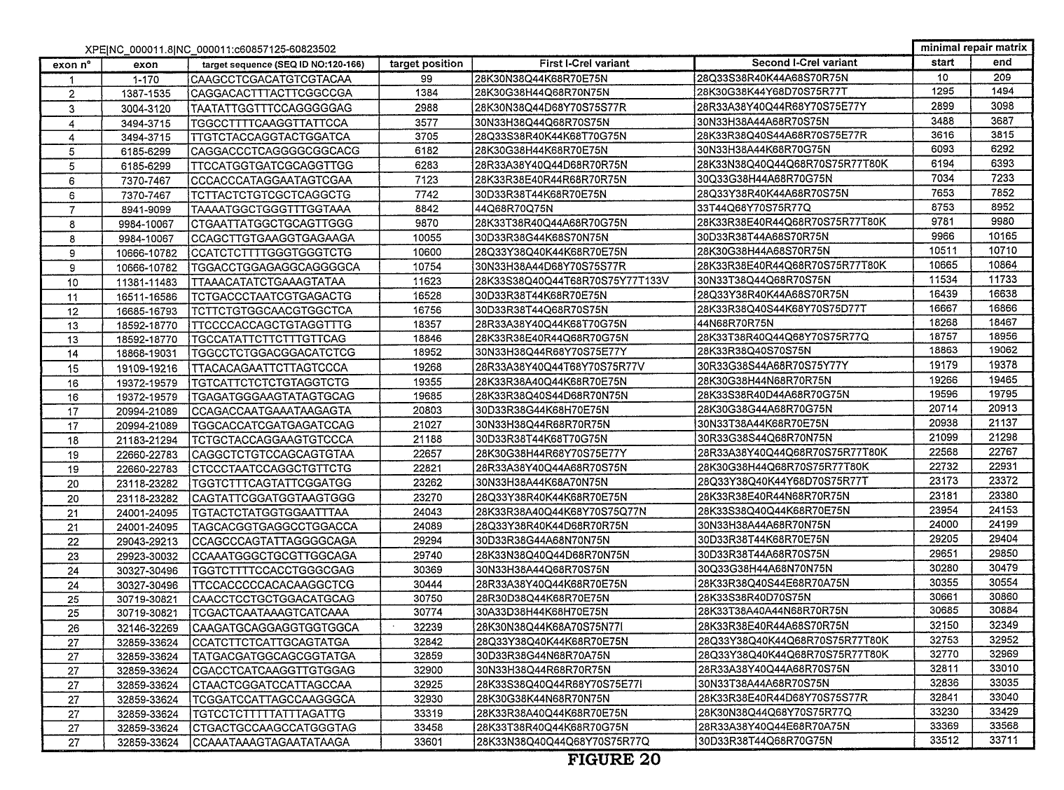

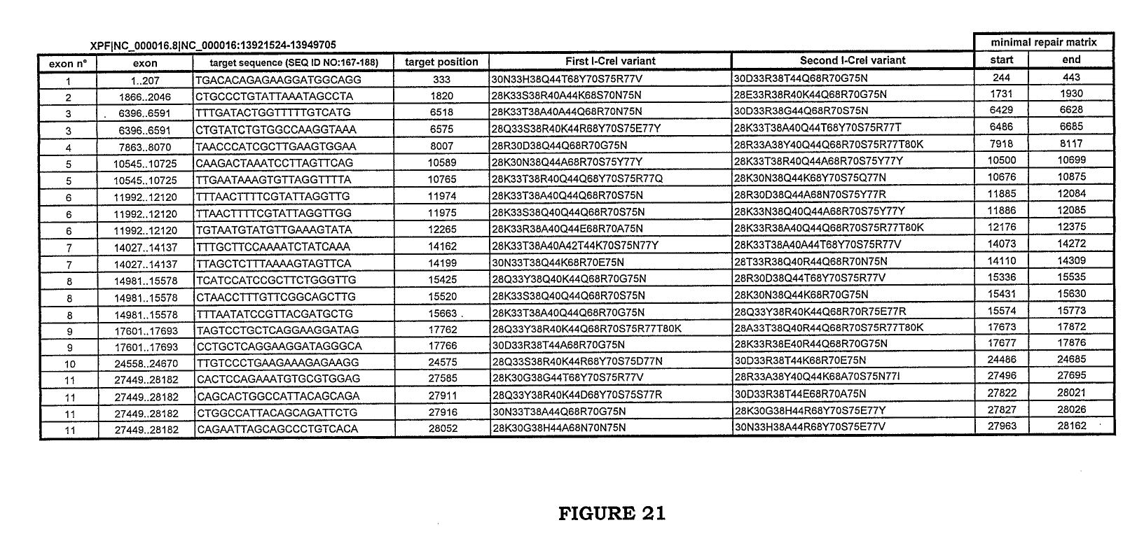

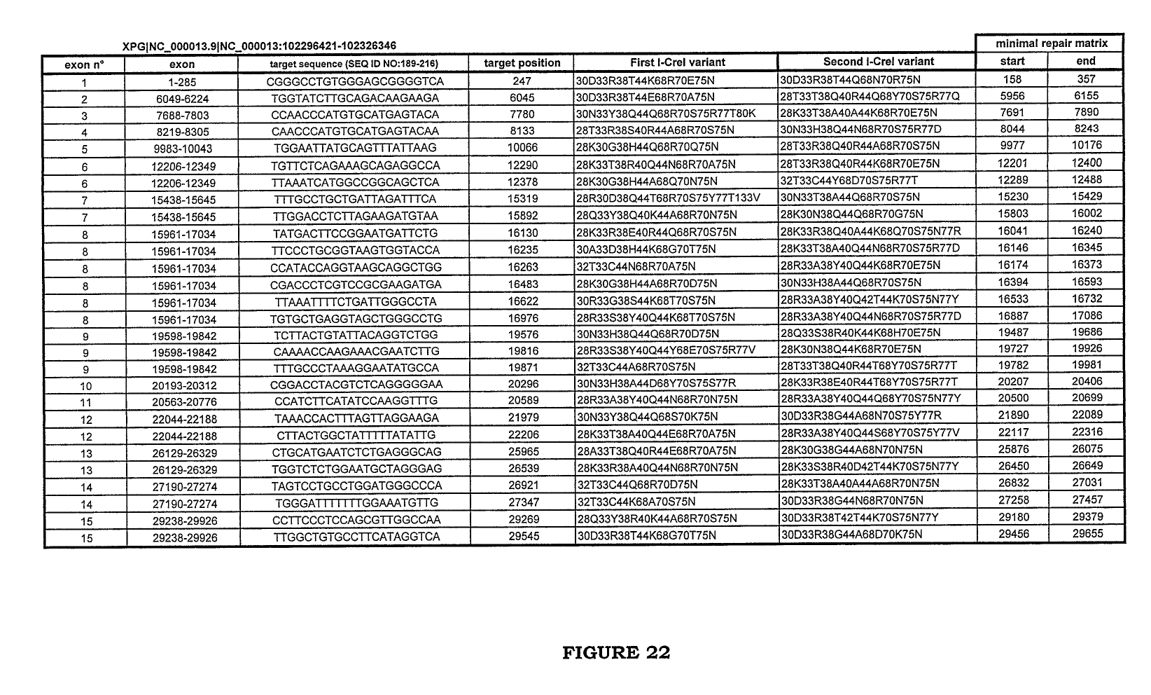

DNA targets from each human XP gene are presented in Tables IX to XV and FIGS. 16 to 22.

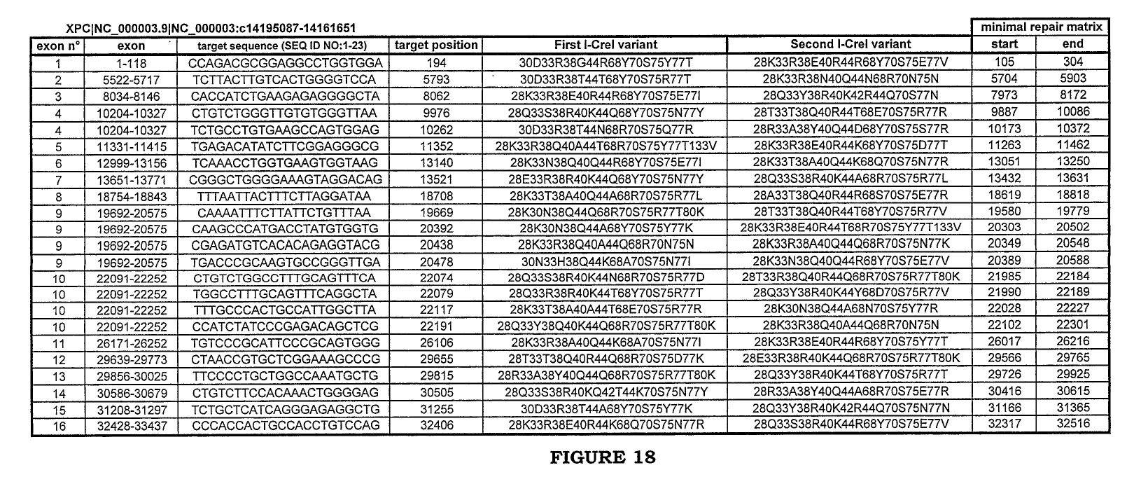

For example, the sequences SEQ ID NO: 1 to 24 are DNA targets from the XPC gene; SEQ ID NO: 1 to 23 are situated in or close to one of the exons and these sequences cover all the exons of the XP gene (Table XI and FIG. 18). The target sequence SEQ ID NO: 24 (Xa.1) is situated in the third intron, upstream of the mutations (FIG. 1A). The target sequence SEQ ID NO: 12 (Xc.1) is situated in Exon 9, in the vicinity of the deletion 1132AA and the insertion insVAL580 (FIG. 1A).

Hererodimeric variants which cleave each DNA target are presented in Tables I to VIII and FIGS. 16 to 22.

The sequence of each variant is defined by its amino acid residues at the indicated positions. For example, the first heterodimeric variant of Table I consists of a first monomer having K, S, R, D, K, R, G and N in positions 28, 33, 38, 40, 44, 68, 70 and 75, respectively and a second monomer having R, D, R, K, A, S, N and I in positions 28, 30, 38, 44, 68, 70, 75 and 77, respectively. The positions are indicated by reference to I-CreI sequence SWISSPROT P05725, SEQ ID NO: 217 or pdb accession code 1 g9y; I-CreI has G, I, Q, K, N, S, Y, Q, S, A, Q, R, D, R, D, I, E, H, F, I, A and S, in positions 19, 24, 26, 28, 30, 32, 33, 38, 40, 42, 44, 68, 69, 70, 75, 77, 80, 85, 87, 109, 133 and 161, respectively. The variant may consist of an I-CreI sequence having the amino acid residues as indicated in the Table. In this case, the positions which are not indicated are not mutated and thus correspond to the wild-type I-CreI sequence. Alternatively, the variant may comprise an I-CreI sequence having the amino acid residues as indicated in the Table. In the latter case, the positions which are not indicated may comprise mutations as defined above, or may not be mutated. For example, the variant may be derived from an I-CreI scaffold protein encoded by SEQ ID NO: 26, said I-CreI scaffold protein (SEQ ID NO: 218) having the insertion of an alanine in position 2, the substitutions A42T, D75N, W110E and R111Q and three additional amino acids (A, A and D) at the C-terminus. In addition, said variant, derived from wild-type I-CreI or an I-CreI scaffold protein, may comprise additional mutations, as defined above.

The target which is cleaved by each heterodimeric variant is indicated in the last column of the Table.

TABLE I
Sequence of heterodimeric I-CreI variants having a DNA
target site in or close to one exon of the XPA gene

| | | Exon closest to the |
|---|---|---|
| | | target sequence |
| First monomer | Second monomer | (SEQ ID NO: 45 to 57) |
| 28K33S38R40D44K68R70G75N | 28R30D38R44K68A70S75N77I | Exon 1 |
| 28K30G38H44Q68R70Q75N | 28K30G38K44Q68R70S75R77T80K | Exon 1 |
| 28K33T38A40Q44N68K70S75R77N | 30D33R38G44N68K70S75R77N | Exon 2 |
| 28K30G38H44R68Y70S75E77Y | 28K33N38Q40Q44K68R70E75N | Exon 3 |
| 28K30N38Q44Q68A70N75N | 28Q33Y38R40K42R44Q70S75N77N | Exon 4 |
| 30N33H38Q44K68R70E75N | 28K33R38A40Q44Q68R70S75N | Exon 4 |
| 28K30N38Q44K68H70E75N | 28K33R38A40Q44Q68R70S75N | Exon 5 |
| 28K30G38G44Q68R70G75N | 30D33R38G44Q68R70S75N | Exon 5 |
| 28K30G38H44Q68R70S75R77T80K | 28K33T38A40Q44Q68R70G75N | Exon 6 |
| 30N33H38Q44Q68A70N75N | 24I26Q28K30N33Y38Q40S44K68R70E75N | Exon 6 |
| 28K30N38Q44K68A70N75N | 28K33R38A40Q44A68R70G75N | Exon 6 |
| 28K33R38E40R44K68S70N75N | 28K30G38H44R68Y70S75E77Y | Exon 6 |
| 28K33R38A40Q44N68R70N75N | 30D33R38G44A68N70N75N | Exon 6 |

TABLE II
Sequence of heterodimeric I-CreI variants having
a DNA target site in or close to one exon of the XPB gene

| | | Exon |
|---|---|---|
| | | closest |
| | | to the |
| | | target |
| | | sequence |
| | | (SEQ ID |
| | | NO: 58 to |
| First monomer | Second monomer | 86) |
| 30R33G38S44K68S70N75N | 30N33H38Q44Q68R70S75R77T80K | exon 1 |
| 30N33H38A44K68R70E75N | 30D33R38T44K68S70N75N | exon 2 |
| 28K33N38Q40Q44R68R70R75N | 28K33R38A40Q44R68R70R75N | exon 3 |
| 28R30D38Q44E68R70A75N | 28Q33S38R40K44K68T70T75N | exon 3 |
| 30N33H38A44A68R70G75N | 28K33T38A40Q68K44Q68Y70S75R77Q | exon 3 |
| 30N33H38A44A68N70N75N | 30N33H38Q44R68Y70S75E77Y | exon 4 |
| 28K33S38Q40Q44R68R70R75N | 28K33R38Q40S44K68R70G75N | exon 5 |
| 28K33T38A40A44Q68R70S75R77T80K | 30N33H38Q44K68Y70S75Q77N | exon 6 |
| 30N33T38A42R44Q70S75N77N | 28R33A38Y40Q44K68R70E75N | exon 7 |
| 30D33R38G44K68A70N75N | 28K30G38H44R68R70R75N | exon 7 |
| 28R33A38Y40Q44Q68R70G75N | 30N33H38A44A68S70R75N | exon 7 |
| 28Q33Y38Q40K44A68N70N75N | 30D33R38G44Q68R70S75R77T80K | exon 8 |
| 30N33H38Q44K68Y70S75D77T | 28Q33Y38Q40K44K68Y70S75D77T | exon 9 |
| 28K33S38Q40Q44Q68R70S75N | 28K30G38H44K68H70E75N | exon 9 |
| 30N33H38A44A68R70S75N | 28K30G38H44A68N70N75N | exon 9 |
| 30D33R38T44K68S70N75N | 30D33R38G44R68Y70S75E77Y | exon 10 |
| 24I28Q28K30N33Y38Q40S44K68H70E75N | 28K30N38Q44K68Y70S75D77T | exon 10 |
| 28K33T38A40A44K68Y70S75Q77N | 30N33H38Q44Q68R70G75N | exon 10 |
| 30N33H38A44N68K70S75R77N | 28R33A38Y40Q44R68Y70S75E77Y | exon 11 |
| 28Q33Y38Q40K44K68T70T75N | 24I26Q28K30N33Y38Q40S44E68R70A75N | exon 11 |
| 24I28Q28K30N33Y38Q40S44Q68R70N75N | 30D33R38T44A68R70S75Y77Y | exon 12 |
| 28K33T38A40Q44Q68R70Q75N | 28K30N38Q44Q68Y70S75R77Q | exon 13 |
| 28K33S38R40D44Y68D70S75R77T | 28K33S38R40D44K68T70T75N | exon 13 |
| 30N33H38A44R68Y70S75E77Y | 28R33A38Y40Q44Q68R70S75R77T80K | exon 14 |
| 28Q33Y38R40K44Q68R70S75R77T80K | 30R33G38S44R68R70R75N | exon 14 |
| 30R33G38S44Q68R70S75N | 28R30D38Q44K68A70N75N | exon 14 |
| 30D33R38T42R44Q70S77N | 30N33T38A44A68R70S75N | exon 15 |
| 30N33T38A44Q68R70S75N | 30N33H38Q44E68R70A75N | exon 15 |
| 30N33H38A44K68A70N75N | 30N33T38Q44A68S70R75N | exon 15 |

TABLE III
Sequence of heterodimeric I-CreI variants having
a DNA target site in or close to one exon of the XPC gene

| | | Exon |
|---|---|---|
| | | closest to |
| | | the target |
| | | sequence |
| | | (SEQ ID |
| First monomer | Second monomer | NO: 1 to 23) |
| 30D33R38G44R68Y70S75Y77T | 28K33R38E40R44R68Y70S75E77V | exon 1 |
| 30D33R38T44T68Y70S75R77T | 28K33R38N40Q44N68R70N75N | exon 2 |
| 28K33R38E40R44R68Y70S75E77I | 28Q33Y38R40K42R44Q70S77N | exon 3 |
| 28Q33S38R40K44Q68Y70S75N77Y | 28T33T38Q40R44T68E70S75R77R | exon 4 |
| 30D33R38T44N68R70S75Q77R | 28R33A38Y40Q44D68Y70S75S77R | exon 4 |
| 28K33R38Q40A44T68R70S75Y77T133V | 28K33R38E40R44K68Y70S75D77T | exon 5 |
| 28K33N38Q40Q44R68Y70S75E77I | 28K33T38A40Q44K68Q70S75N77R | exon 6 |
| 28E33R38R40K44Q68Y70S75N77Y | 28Q33S38R40K44A68R70S75R77L | exon 7 |
| 28K33T38A40Q44A68R70S75R77L | 28A33T38Q40R44R68S70S75E77R | exon 8 |
| 28K30N38Q44Q68R70S75R77T80K | 28T33T38Q40R44T68Y70S75R77V | exon 9 |
| 28K30N38Q44A68Y70S75Y77K | 28K33R38E40R44T68R70S75Y77T133V | exon 9 |
| 28K33R38Q40A44Q68R70N75N | 28K33R38A40Q44Q68R70S75N77K | exon 9 |
| 33H75N | 33R38A40Q44K70N75N | exon 9 |
| 30N33H38Q44K68A70S75N77I | 28K33N38Q40Q44R68Y70S75E77V | exon 9 |
| 28Q33S38R40K44N68R70S75R77D | 28T33R38Q40R44Q68R70S75R77T80K | exon 10 |
| 28Q33R38R40K44T68Y70S75R77T | 28Q33Y38R40K44Y68D70S75R77V | exon 10 |
| 28K33T38A40A44T68E70S75R77R | 28K30N38Q44A68N70S75Y77R | exon 10 |
| 28Q33Y38Q40K44Q68R70S75R77T80K | 28K33R38Q40A44Q68R70N75N | exon 10 |
| 28K33R38A40Q44K68A70S75N77I | 28K33R38E40R44R68Y70S75Y77T | exon 11 |
| 28T33T38Q40R44Q68R70S75D77K | 28E33R38R40K44Q68R70S75R77T80K | exon 12 |
| 28R33A38Y40Q44Q68R70S75R77T80K | 28Q33Y38R40K44T68Y70S75R77T | exon 13 |
| 28Q33S38R40K42T44K70S75N77Y | 28R33A38Y40Q44A68R70S75E77R | exon 14 |
| 30D33R38T44A68Y70S75Y77K | 28Q33Y38R40K42R44Q70S75N77N | exon 15 |
| 28K33R38E40R44K68Q70S75N77R | 28Q33S38R40K44R68Y70S75E77V | exon 16 |

TABLE IV
Sequence of heterodimeric I-CreI variants having
a DNA target site in or close to one exon of the XPD gene

| | | Exon closest |
|---|---|---|
| | | to the target |
| | | sequence |
| | | (SEQ ID NO: |
| First monomer | Second monomer | 87 to 119) |
| 30A33D38H44K68R70E75N | 28K30G38K44K68S70N75N | exon 1 |
| 30N33T38Q44Q68R70G75N | 30N33H38Q44A68R70S75E77R | exon 2 |
| 30N33H38A44N68R70N75N | 30N33H38Q44Q68R70S75R77T80K | exon 3 |
| 28K33R38E40R44E68R70A75N | 28R33A38Y40Q44A68S70R75N | exon 3 |
| 28K33T38R40Q44K68R70E75N | 28Q33S38R40K44K68R70G75N | exon 4 |
| 30D33R38T44K68R70E75N | 28K30G38G44Q68Y70S75R77Q | exon 5 |
| 30D33R38T44K68R70E75N | 30R33G38S44A68R70S75N | exon 5 |
| 28K30G38H44A68R70N75N | 28K33S38R40D44A68N70N75N | exon 6 |
| 30N33H38Q44Y68D70S75R77T | 28R30D38R44A68N70N75N | exon 7 |
| 28K33R38Q40S44K68T70T75N | 30D33R38G44K68R70E75N | exon 7 |
| 28R33A38Y40Q44Q68R70Q75N | 28K30G38H44R68Y70S75E77Y | exon 8 |
| 33R38E44Q68R70G75N | 28K33R38E40R44K68R70E75N | exon 8 |
| 30R33G38S44K68R70E75N | 30D33R38G44A68R70N75N | exon 9 |
| 30R33G38S44A68R70S75N | 30D33R38G44K68R70G75N | exon 10 |
| 30N33H38A44K68R70G75N | 28Q33Y38R40K44K68A70N75N | exon 10 |
| 30N33T38A44N68R70N75N | 28K30G38K44Q68R70S75N | exon 11 |
| 28R33A38Y40Q44K68R70G75N | 28Q33Y38R40K44T68Y70S75R77V | exon 12 |
| 28Q33Y38R40K44K68Y70S75Q77N | 33T44Q68R70S75N | exon 12 |
| 28K30G38G44A68N70N75N | 28K33R38E40R44E68R70A75N | exon 13 |
| 28K33R38A40Q44R68R70R75N | 28K33R38E40R44T68Y70S75R77V | exon 14 |
| 28K33R38Q40S44K68R70E75N | 30R33G38S44A68R70S75E77R | exon 15 |
| 28Q33Y38R40K44D68R70N75N | 28K33R38A40Q44R68R70R75N | exon 16 |
| 30N33H38A44Q68R70G75N | 28Q33Y38R40K44A68S70R75N | exon 16 |
| 30D33R38T44A68R70N75N | 28K33R38E40R44A68R70S75N | exon 17 |
| 28Q33Y38R40K44E68R70A75N | 33R38E44N68R70N75N | exon 17 |
| 28K30G38H44A68R70S75N | 30N33T38Q44R68Y70S75E77Y | exon 17 |
| 28K33R38E40R44Q68R70G75N | 30N33T38A44R68R70R75N | exon 18 |
| 28Q33Y38Q40K44Q68R70S75R77T80K | 30R33G38S44G68Q70T75N | exon 19 |
| 30N33H38Q44D68R70N75N | 28K30G38K44G68Q70T75N | exon 19 |
| 28K33N38Q40Q44A68N70N75N | 28K30G38H44Q68R70G75N | exon 20 |
| 28K33R38E40R44K68R70E75N | 28K30G38H44A68R70S75N | exon 22 |
| 28Q33S38R40K44K68R70E75N | 28K33T38A40A44A68R70S75N | exon 22 |
| 28K33N38Q40Q44K68T70T75N | 28K30G38H44Q68A70N75N | exon 23 |

TABLE V
Sequence of heterodimeric I-CreI variants having a DNA target site in
or close to one exon of the XPE gene

| | | Exon closest to the target |
|---|---|---|
| First monomer | Second monomer | sequence (SEQ ID NO: 120 to 166) |
| 28K30N38Q44K68R70E75N | 28Q33S38R40K44A68S70R75N | exon 1 |
| 28K30G38H44Q68R70N75N | 28K30G38K44Y68D70S75R77T | exon 2 |
| 28K30N38Q44D68Y70S75S77R | 28R33A38Y40Q44R68Y70S75E77Y | exon 3 |
| 30N33H38Q44Q68R70S75N | 30N33H38A44A68R70S75N | exon 4 |
| 28Q33S38R40K44K68T70G75N | 28K33R38Q40S44A68R70S75E77R | exon 4 |
| 28K30G38H44K68R70E75N | 30N33H38A44K68R70G75N | exon 5 |
| 28R33A38Y40Q44D68R70R75N | 28K33N38Q40Q44Q68R70S75R77T80K | exon 5 |
| 28K33R38E40R44R68R70R75N | 30Q33G38H44A68R70G75N | exon 6 |
| 30D33R38T44K68R70E75N | 28Q33Y38R40K44A68R70S75N | exon 6 |
| 44Q68R70Q75N | 33T44Q68Y70S75R77Q | exon 7 |
| 28K33T38R40Q44A68R70G75N | 28K33R38E40R44Q68R70S75R77T80K | exon 8 |
| 30D33R38G44K68S70N75N | 30D33R38T44A68S70R75N | exon 8 |
| 28Q33Y38Q40K44K68R70E75N | 28K30G38H44A68S70R75N | exon 9 |
| 30N33H38A44D68Y70S75S77R | 28K33R38E40R44Q68R70S75R77T80K | exon 9 |
| 28K33S38Q40Q44T68R70S75Y77T133V | 30N33T38Q44Q68R70S75N | exon 10 |
| 30D33R38T44K68R70E75N | 28Q33Y38R40K44A68S70R75N | exon 11 |
| 30D33R38T44Q68R70S75N | 28K33R38Q40S44K68Y70S75D77T | exon 12 |
| 28R33A38Y40Q44K68T70G75N | 44N68R70R75N | exon 13 |
| 28K33R38E40R44Q68R70G75N | 28K33T38R40Q44Q68Y70S75R77Q | exon 13 |
| 30N33H38Q44R68Y70S75E77Y | 28K33R38Q40S70S75N | exon 14 |
| 28R33A38Y40Q44T68Y70S75R77V | 30R33G38S44A68R70S75Y77Y | exon 15 |
| 28K33R38A40Q44K68R70E75N | 28K30G38H44N68R70R75N | exon 16 |
| 28K33R38Q40S44D68R70N75N | 28K33S38R40D44A68R70G75N | exon 16 |
| 30D33R38G44K68H70E75N | 28K30G38G44A68R70G75N | exon 17 |
| 30N33H38Q44R68R70R75N | 30N33T38A44K68R70E75N | exon 17 |
| 30D33R38T44K68T70G75N | 30R33G38S44Q68R70N75N | exon 18 |
| 28K30G38H44R68Y70S75E77Y | 28R33A38Y40Q44Q68R70S75R77T80K | exon 19 |
| 28R33A38Y40Q44A68R70S75N | 28K30G38H44Q68R70S75R77T80K | exon 19 |
| 30N33H38A44K68A70N75N | 28Q33Y38Q40K44Y68D70S75R77T | exon 20 |
| 28Q33Y38R40K44K68R70E75N | 28K33R38E40R44N68R70R75N | exon 20 |
| 28K33R38A40Q44K68Y70S75Q77N | 28K33S38Q40Q44K68R70E75N | exon 21 |
| 28Q33Y38R40K44D68R70R75N | 30N33H38A44A68R70N75N | exon 21 |
| 30D33R38G44A68N70N75N | 30D33R38T44K68R70E75N | exon 22 |
| 28K33N38Q40Q44D68R70N75N | 30D33R38T44A68R70S75N | exon 23 |
| 30N33H38A44Q68R70S75N | 30Q33G38H44A68N70N75N | exon 24 |
| 28R33A38Y40Q44K68R70E75N | 28K33R38Q40S44E68R70A75N | exon 24 |
| 28R30D38Q44K68R70E75N | 28K33S38R40D70S75N | exon 25 |
| 30A33D38H44K68H70E75N | 28K33T38A40A44N68R70R75N | exon 25 |
| 28K30N38Q44K68A70S75N77I | 28K33R38E40R44A68S70R75N | exon 26 |
| 28Q33Y38Q40K44K68R70E75N | 28Q33Y38Q40K44Q68R70S75R77T80K | exon 27 |
| 30D33R38G44N68R70A75N | 28Q33Y38Q40K44Q68R70S75R77T80K | exon 27 |
| 30N33H38Q44R68R70R75N | 28R33A38Y40Q44A68R70S75N | exon 27 |
| 28K33S38Q40Q44R68Y70S75E77I | 30N33T38A44A68R70S75N | exon 27 |
| 28K30G38K44N68R70N75N | 28K33R38E40R44D68Y70S75S77R | exon 27 |
| 28K33R38A40Q44K68R70E75N | 28K30N38Q44Q68Y70S75R77Q | exon 27 |
| 28K33T38R40Q44K68R70G75N | 28R33A38Y40Q44E68R70A75N | exon 27 |
| 28K33N38Q40Q44Q68Y70S75R77Q | 30D33R38T44Q68R70G75N | exon 27 |

TABLE VI
Sequence of heterodimeric I-CreI variants having
a DNA target site in or close to one exon of the XPF gene

| | | Exon closest |
|---|---|---|
| | | to the target |
| | | sequence |
| | | (SEQ ID NO: |
| First monomer | Second monomer | 167 to 188) |
| 30N33H38Q44T68Y70S75R77V | 30D33R38T44Q68R70G75N | exon 1 |
| 28K33S38R40A44K68S70N75N | 28E33R38R40K44Q68R70G75N | exon 2 |
| 28K33T38A40A44Q68R70N75N | 30D33R38G44Q68R70S75N | exon 3 |
| 28Q33S38R40K44R68Y70S75E77Y | 28K33T38A40Q44T68Y70S75R77T | exon 3 |
| 28R30D38Q44Q68R70G75N | 28R33A38Y40Q44Q68R70S75R77T80K | exon 4 |
| 28K30N38Q44A68R70S75Y77Y | 28K33T38R40Q44A68R70S75Y77Y | exon 5 |
| 28K33T38R40Q44Q68Y70S75R77Q | 28K30N38Q44K68Y70S75Q77N | exon 5 |
| 28K33T38A40Q44Q68R70S75N | 28R30D38Q44A68N70S75Y77R | exon 6 |
| 28K33S38Q40Q44Q68R70S75N | 28K33N38Q40Q44A68R70S75Y77Y | exon 6 |
| 28K33R38A40Q44E68R70A75N | 28K33R38A40Q44Q68R70S75R77T80K | exon 6 |
| 28K33T38A40A42T44K70S75N77Y | 28K33T38A40A44T68Y70S75R77V | exon 7 |
| 30N33T38Q44K68R70E75N | 28T33R38Q40R44Q68R70N75N | exon 7 |
| 28Q33Y38Q40K44Q68R70G75N | 28R30D38Q44T68Y70S75R77V | exon 8 |
| 28K33S38Q40Q44Q68R70S75N | 28K30N38Q44K68R70G75N | exon 8 |
| 28K33T38A40Q44Q68R70G75N | 28Q33Y38R40K44Q68R70R75E77R | exon 8 |
| 28Q33Y38R40K44Q68R70S75R77T80K | 28A33T38Q40R44Q68R70S75R77T80K | exon 9 |
| 30D33R38T44A68R70G75N | 28K33R38E40R44Q68R70G75N | exon 9 |
| 28Q33S38R40K44R68Y70S75D77N | 30D33R38T44K68R70E75N | exon 10 |
| 28K30G38G44T68Y70S75R77V | 28R33A38Y40Q44K68A70S75N77I | exon 11 |
| 28Q33Y38R40K44D68Y70S75S77R | 30D33R38T44E68R70A75N | exon 11 |
| 30N33T38A44Q68R70G75N | 28K30G38H44R68Y70S75E77Y | exon 11 |
| 28K30G38H44A68N70N75N | 30N33H38A44R68Y70S75E77V | exon 11 |

TABLE VII
Sequence of heterodimeric I-CreI variants having
a DNA target site in or close to one exon of the XPG gene

| | | Exon closest to |
|---|---|---|
| | | the target |
| | | sequence |
| | | (SEQ ID NO: |
| First monomer | Second monomer | 189 to 216) |
| 30D33R38T44K68R70E75N | 30D33R38T44Q68N70R75N | exon 1 |
| 30D33R38T44E68R70A75N | 28T33T38Q40R44Q68Y70S75R77Q | exon 2 |
| 30N33Y38Q44Q68R70S75R77T80K | 28K33T38A40A44K68R70E75N | exon 3 |
| 28T33R38S40R44A68R70S75N | 30N33H38Q44N68R70S75R77D | exon 4 |
| 28K30G38H44Q68R70Q75N | 28T33R38Q40R44A68R70S75N | exon 5 |
| 28K33T38R40Q44N68R70A75N | 28T33R38Q40R44K68R70E75N | exon 6 |
| 28K30G38H44A68Q70N75N | 32T33C44Y68D70S75R77T | exon 6 |
| 28R30D38Q44T68R70S75Y77T133V | 30N33T38A44Q68R70S75N | exon 7 |
| 28Q33Y38Q40K44A68R70N75N | 28K30N38Q44Q68R70G75N | exon 7 |
| 28K33R38E40R44Q68R70S75N | 28K33R38Q40A44K68Q70S75N77R | exon 8 |
| 30A33D38H44K68G70T75N | 28K33T38A40Q44N68R70S75R77D | exon 8 |
| 32T33C44N68R70A75N | 28R33A38Y40Q44K68R70E75N | exon 8 |
| 28K30G38H44A68R70D75N | 30N33H38A44Q68R70S75N | exon 8 |
| 30R33G38S44K68T70S75N | 28R33A38Y40Q42T44K70S75N77Y | exon 8 |
| 28R33S38Y40Q44K68T70S75N | 28R33A38Y40Q44N68R70S75R77D | exon 8 |
| 30N33H38Q44Q68R70D75N | 28Q33S38R40K44K68H70E75N | exon 9 |
| 28R33S38Y40Q44Y68E70S75R77V | 28K30N38Q44K68R70E75N | exon 9 |
| 32T33C44A68R70S75N | 28T33T38Q40R44T68Y70S75R77T | exon 9 |
| 30N33H38A44D68Y70S75S77R | 28K33R38E40R44T68Y70S75R77T | exon 10 |
| 28R33A38Y40Q44N68R70N75N | 28R33A38Y40Q44Q68Y70S75N77Y | exon 11 |
| 30N33Y38Q44Q68S70K75N | 30D33R38G44A68N70S75Y77R | exon 12 |
| 28K33T38A40Q44E68R70A75N | 28R33A38Y40Q44S68Y70S75Y77V | exon 12 |
| 28A33T38Q40R44E68R70A75N | 28K30G38G44A68N70N75N | exon 13 |
| 28K33R38A40Q44N68R70N75N | 28K33S38R40D42T44K70S75N77Y | exon 13 |
| 32T33C44Q68R70D75N | 28K33T38A40A44A68R70N75N | exon 14 |
| 32T33C44K68A70S75N | 30D33R38G44N68R70N75N | exon 14 |
| 28Q33Y38R40K44A68R70S75N | 30D33R38T42T44K70S75N77Y | exon 15 |
| 30D33R38T44K68G70T75N | 30D33R38G44A68D70K75N | exon 15 |

TABLE VIII
Sequence of heterodimeric I-CreI variants having a DNA target site
(SEQ ID NO: 24) situated in the third intron of the XPC gene

| First monomer | Second monomer |
|---|---|
| 28K30N33S38R40S70S75N | 28E30N33Y38R40K44K68S70S75N |
| 28A30N33S38R40K70S75N | 28K30G33Y38R40S44K68R70E75N |
| 19A28A30N33S38R40K70S75N | 28E30N33Y38R40K44K68R70E75N85R109T |
| 19A28A30N33Y38R40K70S75N87L | 28E30N33Y38R40K44K68R70E75N85R109T161F |
| 19A28A30N33S38R40K69G70S75N | 28E30N32R33Y38Q40K44K68R70E75N85R109T |
| | 28S30N33Y38R40K44K68S70S75N |
| | 28S30N33Y38R40K44K68R70D75N |
| | 28S30N33Y38R40K44K68A70S75N |
| | 28K30G33Y38H40S44K68R70E75N |
| | 28K30G33Y38H40S44K68A70G75N |
| | 28K30G33Y38R40S44K68R70E75N |
| | 28K30G33Y38R40S44K68T70H75N |
| | 28K30G33Y38R40S44K68S70S75N |
| | 28K30G33Y38R40S44K68T70S75N |

In addition, the variants of the invention may include one or more residues inserted at the NH₂ terminus and/or COOH terminus of the sequence. For example, a tag (epitope or polyhistidine sequence) is introduced at the NH₂ terminus and/or COOH terminus; said tag is useful for the detection and/or the purification of said variant.

The subject-matter of the present invention is also a single-chain chimeric meganuclease derived from an I-CreI variant as defined above. The single-chain chimeric meganuclease is a fusion protein comprising two I-CreI monomers, two I-CreI core domains (positions 6 to 94 of I-CreI) or a combination of both. Preferably, the two monomers/core domains or the combination of both are connected by a peptidic linker.

The subject-matter of the present invention is also a polynucleotide fragment encoding a variant or a single-chain chimeric meganuclease as defined above; said polynucleotide may encode one monomer of an homodimeric or heterodimeric variant, or two domains/monomers of a single-chain chimeric meganuclease.

The subject-matter of the present invention is also a recombinant vector for the expression of a variant or a single-chain meganuclease according to the invention. The recombinant vector comprises at least one polynucleotide fragment encoding a variant or a single-chain meganuclease, as defined above. In a preferred embodiment, said vector comprises two different polynucleotide fragments, each encoding one of the monomers of an heterodimeric variant.

A vector which can be used in the present invention includes, but is not limited to, a viral vector, a plasmid, a RNA vector or a linear or circular DNA or RNA molecule which may consists of a chromosomal, non chromosomal, semi-synthetic or synthetic DNA. Preferred vectors are those capable of autonomous repli-cation (episomal vector) and/or expression of nucleic acids to which they are linked (expression vectors). Large numbers of suitable vectors are known to those of skill in the art and commercially available.

Viral vectors include retrovirus, adenovirus, parvovirus (e.g. adeno-associated viruses), coronavirus, negative strand RNA viruses such as orthomyxovirus (e.g., influenza virus), rhabdovirus (e.g., rabies and vesicular stomatitis virus), para-myxovirus (e.g. measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields, et al., Eds., Lippincott-Raven Publishers, Philadelphia, 1996).

Vectors can comprise selectable markers, for example: neomycin phosphotransferase, histidinol dehydrogenase, dihydrofolate reductase, hygromycin phosphotransferase, herpes simplex virus thymidine kinase, adenosine deaminase, glutamine synthetase, and hypoxanthine-guanine phosphoribosyl transferase for eukaryotic cell culture; TRP1 for S. cerevisiae; tetracycline, rifampicin or ampicillin resistance in E. coli.

Preferably said vectors are expression vectors, wherein the sequence(s) encoding the variant/single-chain meganuclease of the invention is placed under control of appropriate transcriptional and translational control elements to permit production or synthesis of said variant. Therefore, said polynucleotide is comprised in an expression cassette. More particularly, the vector comprises a replication origin, a promoter operatively linked to said encoding polynucleotide, a ribosome-binding site, an RNA-splicing site (when genomic DNA is used), a polyadenylation site and a transcription termination site. It also can comprise an enhancer. Selection of the promoter will depend upon the cell in which the poly-peptide is expressed. Preferably, when said variant is an heterodimer, the two poly-nucleotides encoding each of the monomers are included in one vector which is able to drive the expression of both polynucleotides, simultaneously. Suitable promoters include tissue specific and/or inducible promoters. Examples of inducible promoters are: eukaryotic metallothionine promoter which is induced by increased levels of heavy metals, prokaryotic lacZ promoter which is induced in response to isopropyl-β-D-thiogalacto-pyranoside (IPTG) and eukaryotic heat shock promoter which is induced by increased temperature. Examples of tissue specific promoters are skeletal muscle creatine kinase, prostate-specific antigen (PSA), α-antitrypsin protease, human surfactant (SP) A and B proteins, β-casein and acidic whey protein genes.

According to another advantageous embodiment of said vector, it includes a targeting construct comprising sequences sharing homologies with the region surrounding the genomic DNA target cleavage site as defined above.

Alternatively, the vector coding for a I-CreI variant and the vector comprising the targeting construct are different vectors.

In both cases, the targeting construct comprises a sequence to be introduced flanked by sequences sharing homologies with the regions surrounding the genomic DNA cleavage sites of the variant as defined here after.

More preferably, said targeting DNA construct comprises:

a) sequences sharing homologies with the region surrounding the genomic DNA cleavage site as defined above, and

b) a sequence to be introduced flanked by sequences as in a).

Preferably, homologous sequences of at least 50 bp, preferably more than 100 bp and more preferably more than 200 bp are used. Indeed, shared DNA homologies are located in regions flanking upstream and downstream the site of the break and the DNA sequence to be introduced should be located between the two arms. The sequence to be introduced is preferably a sequence which repairs a mutation in the gene of interest (gene correction or recovery of a functional gene), for the purpose of genome therapy. Alternatively, it can be any other sequence used to alter the chromosomal DNA in some specific way including a sequence used to modify a specific sequence, to attenuate or activate the endogenous gene of interest, to inactivate or delete the endogenous gene of interest or part thereof, to introduce a mutation into a site of interest or to introduce an exogenous gene or part thereof. Such chromosomal DNA alterations are used for genome engineering (animal models).

For correcting the XP gene, cleavage of the gene occurs in the vicinity of the mutation, preferably, within 500 bp of the mutation (FIG. 1B). The targeting construct comprises a XP gene fragment which has at least 200 bp of homologous sequence flanking the target site (minimal repair matrix) for repairing the cleavage, and includes the correct sequence of the XP gene for repairing the mutation (FIG. 1B). Consequently, the targeting construct for gene correction comprises or consists of the minimal repair matrix; it is preferably from 200 pb to 6000 pb, preferably from 1000 pb to 2000 pb.

For example, the target which is cleaved by each of the variant (Tables I to VIII) and the minimal matrix for repairing the cleavage with each variant are indicated in Tables IX to XV and in FIGS. 16 to 22.

TABLE IX
XPA gene targets cleaved by I-CreI variants

| | | | |
|---|---|---|---|
| Exon closest | Target | | minimal repair |
| to the target | SEQ ID | target | matrix |

| | | | | | | |
|---|---|---|---|---|---|---|
| sequence | Exon position | NO: | target sequence | position | start | end |

| | | | | | | |
|---|---|---|---|---|---|---|
| Exon 1 | 1-237 | 45 | ctgcctgcctcggtgcgggcga | 123 | 34 | 233 |
| Exon 1 | 1-237 | 46 | cagactggctcgtgcaagccga | 290 | 201 | 400 |
| Exon 2 | 3599-3709 | 47 | tttacttacttttgtaggcatg | 3582 | 3493 | 3692 |
| Exon 3 | 7719-7824 | 48 | taggacctgttatggaatttga | 7716 | 7627 | 7826 |
| Exon 4 | 10097-10262 | 49 | taatctgttttcagagatgctg | 10083 | 9994 | 10193 |
| Exon 4 | 10097-10262 | 50 | tgaaactctacttaaagttaca | 10240 | 10151 | 10350 |
| Exon 5 | 12318-12435 | 51 | taagctcaaacctcacagtacg | 12602 | 12513 | 12712 |
| Exon 5 | 12318-12435 | 52 | tacgacatttctgaaaagcatg | 12620 | 12531 | 12730 |
| Exon 6 | 21771-22448 | 53 | cagaattgcggcgagcagtaag | 21768 | 21679 | 21878 |
| Exon 6 | 21771-22448 | 54 | tgacctgttttatagaatttta | 21933 | 21844 | 22043 |
| Exon 6 | 21771-22448 | 55 | taatacttcagtaataattatg | 22044 | 21955 | 22154 |
| Exon 6 | 21771-22448 | 56 | caccactgtaccccaggttcta | 22317 | 22228 | 22427 |
| Exon 6 | 21771-22448 | 57 | tgtaccccaggttctagtcatg | 22323 | 22234 | 22433 |

TABLE X
XPB gene targets cleaved by I-CreI variants

| | | | | |
|---|---|---|---|---|
| Exon | | | | |
| closest to | | | | minimal repair |
| the target | Exon | Target | target | matrix |

| | | | | | | |
|---|---|---|---|---|---|---|
| sequence | Position | SEQ ID NO | target sequence | position | start | end |

| | | | | | | |
|---|---|---|---|---|---|---|
| Exon 1 | 1-123 | 58 | cgggcctgtgggagcggggtca | 49 | −40 | 159 |
| Exon 2 | 459-664 | 59 | tggtatcttgcagacaagaaga | 446 | 357 | 556 |
| Exon 3 | 1331-1567 | 60 | ccaacccatgtgcatgagtaca | 1424 | 1335 | 1534 |
| Exon 3 | 1331-1567 | 61 | caacccatgtgcatgagtacaa | 1425 | 1336 | 1535 |
| Exon 3 | 1331-1567 | 62 | tggaattatgcagtttattaag | 1546 | 1457 | 1656 |
| Exon 4 | 3904-3953 | 63 | tgttctcagaaagcagaggcca | 3713 | 3624 | 3823 |
| Exon 5 | 4353-4488 | 64 | ttaaatcatggccggcagctca | 4197 | 4108 | 4307 |
| Exon 6 | 4676-4840 | 65 | tttgcctgctgattagatttca | 5104 | 5015 | 5214 |
| Exon 7 | 5313-5517 | 66 | ttggacctcttagaagatgtaa | 5237 | 5148 | 5347 |
| Exon 7 | 5313-5517 | 67 | tatgacttccggaatgattctg | 5373 | 5284 | 5483 |
| Exon 7 | 5313-5517 | 68 | ttccctgcggtaagtggtacca | 5509 | 5420 | 5619 |
| Exon 8 | 7160-7474 | 69 | ccataccaggtaagcaggctgg | 7466 | 7377 | 7576 |
| Exon 9 | 13546-13730 | 70 | cgaccctcgtccgcgaagatga | 13606 | 13517 | 13716 |
| Exon 9 | 13546-13730 | 71 | ttaaattttctgattgggccta | 13641 | 13552 | 13751 |
| Exon 9 | 13546-13730 | 72 | tgtgctgaggtagctgggcctg | 13722 | 13633 | 13832 |
| Exon 10 | 14802-15004 | 73 | tcttactgtattacaggtctgg | 14786 | 14697 | 14896 |
| Exon 10 | 14802-15004 | 74 | caaaaccaagaaacgaatcttg | 14849 | 14760 | 14959 |
| Exon 10 | 14802-15004 | 75 | tttgccctaaaggaatatgcca | 14970 | 14881 | 15080 |
| Exon 11 | 21216-21312 | 76 | cggacctacgtctcagggggaa | 21228 | 21139 | 21338 |
| Exon 11 | 21216-21312 | 77 | ccatcttcatatccaaggtttg | 21296 | 21207 | 21406 |
| Exon 12 | 22724-22841 | 78 | taaaccactttagttaggaaga | 22885 | 22796 | 22995 |
| Exon 13 | 32831-32849 | 79 | cttactggctatttttatattg | 32467 | 32378 | 32577 |
| Exon 13 | 32831-32849 | 80 | ctgcatgaatctctgagggcag | 33092 | 33003 | 33202 |
| Exon 14 | 34729-34881 | 81 | tggtctctggaatgctagggag | 34570 | 34481 | 34680 |
| Exon 14 | 34729-34881 | 82 | tagtcctgcctggatgggccca | 34958 | 34869 | 35068 |
| Exon 14 | 34729-34881 | 83 | tgggatttttttggaaatgttg | 34980 | 34891 | 35090 |
| Exon 15 | 36450-36887 | 84 | ccttccctccagcgttggccaa | 36673 | 36584 | 36783 |
| Exon 15 | 36450-36887 | 85 | ttggctgtgccttcataggtca | 36723 | 36634 | 36833 |
| Exon 15 | 36450-36887 | 86 | tgtgccttcataggtcatctag | 36728 | 36639 | 36838 |

TABLE XI
XPC gene targets cleaved by I-CreI variants

| | | | | |
|---|---|---|---|---|
| Exon | | | | |
| closest to | | Target | | minimal repair |
| the target | Exon | SEQ ID | target | matrix |

| | | | | | | |
|---|---|---|---|---|---|---|
| sequence | position | NO | target sequence | position | start | end |

| | | | | | | |
|---|---|---|---|---|---|---|
| Exon 1 | 1-118 | 1 | ccagacgcggaggcctggtgga | 194 | 105 | 304 |
| Exon 2 | 5522-5717 | 2 | tcttacttgtcactggggtcca | 5793 | 5704 | 5903 |
| Exon 3 | 8034-8146 | 3 | caccatctgaagagaggggcta | 8062 | 7973 | 8172 |
| Exon 4 | 10204-10327 | 4 | ctgtctgggttgtgtgggttaa | 9976 | 9887 | 10086 |
| Exon 4 | 10204-10327 | 5 | tctgcctgtgaagccagtggag | 10262 | 10173 | 10372 |
| Exon 5 | 11331-11415 | 6 | tgagacatatcttcggagggcg | 11352 | 11263 | 11462 |
| Exon 6 | 12999-13156 | 7 | tcaaacctggtgaagtggtaag | 13140 | 13051 | 13250 |
| Exon 7 | 13651-13771 | 8 | cgggctggggaaagtaggacag | 13521 | 13432 | 13631 |
| Exon 8 | 18754-18843 | 9 | tttaattactttcttaggataa | 18708 | 18619 | 18818 |
| Exon 9 | 19692-20575 | 10 | caaaatttcttattctgtttaa | 19669 | 19580 | 19779 |
| Exon 9 | 19692-20575 | 11 | caagcccatgacctatgtggtg | 20392 | 20303 | 20502 |
| Exon 9 | 19692-20575 | 12 | cgagatgtcacacagaggtacg | 20438 | 20349 | 20548 |
| Exon 9 | 19692-20575 | 13 | tgacccgcaagtgccgggttga | 20478 | 20389 | 20588 |
| Exon 10 | 22091-22252 | 14 | ctgtctggcctttgcagtttca | 22074 | 21985 | 22184 |
| Exon 10 | 22091-22252 | 15 | tggcctttgcagtttcaggcta | 22079 | 21990 | 22189 |
| Exon 10 | 22091-22252 | 16 | tttgcccactgccattggctta | 22117 | 22028 | 22227 |
| Exon 10 | 22091-22252 | 17 | ccatctatcccgagacagctcg | 22191 | 22102 | 22301 |
| Exon 11 | 26171-26252 | 18 | tgtcccgcattcccgcagtggg | 26106 | 26017 | 26216 |
| Exon 12 | 29639-29773 | 19 | ctaaccgtgctcggaaagcccg | 29655 | 29566 | 29765 |
| Exon 13 | 29856-30025 | 20 | ttcccctgctggccaaatgctg | 29815 | 29726 | 29925 |
| Exon 14 | 30586-30679 | 21 | ctgtcttccacaaactggggag | 30505 | 30416 | 30615 |
| Exon 15 | 31208-31297 | 22 | tctgctcatcagggagaggctg | 31255 | 31166 | 31365 |
| Exon 16 | 32428-33437 | 23 | cccaccactgccacctgtccag | 32406 | 32317 | 32516 |

TABLE XII
XPD gene targets cleaved by I-CreI variants

| | | | | |
|---|---|---|---|---|
| Exon | | | | |
| closest to | | Target | | Minimal |
| the target | Exon | SEQ ID | target | repair matrix |

| | | | | | | |
|---|---|---|---|---|---|---|
| sequence | Position | NO | target sequence | position | start | end |

| | | | | | | |
|---|---|---|---|---|---|---|
| Exon 1 | 1-36 | 87 | tcgaccccgctgcacagtccgg | 2 | −87 | 112 |
| Exon 2 | 340-439 | 88 | ctatatatcagctactgtgcca | 901 | 812 | 1011 |
| Exon 3 | 1425-1502 | 89 | tggtccccaacatgcagggtca | 1408 | 1319 | 1518 |
| Exon 3 | 1425-1502 | 90 | cccaacatgcagggtcatggag | 1413 | 1324 | 1523 |
| Exon 4 | 1580-1642 | 91 | ctgaacccgtaaaggcagacaa | 1515 | 1426 | 1625 |
| Exon 5 | 1829-1942 | 92 | cctgccccccaactttggagta | 1806 | 1717 | 1916 |
| Exon 5 | 1829-1942 | 93 | ccttctccttgcccttagccca | 1956 | 1867 | 2066 |
| Exon 6 | 5414-5530 | 94 | caggacaggtagcctggggcag | 5562 | 5473 | 5672 |
| Exon 7 | 5618-5734 | 95 | tgacctgaaggccctggggcgg | 5674 | 5585 | 5784 |
| Exon 7 | 5618-5734 | 96 | cgatactoagtgaggaggctgg | 5726 | 5637 | 5836 |
| Exon 8 | 6025-6148 | 97 | otccccggccccccagatcctg | 6009 | 5920 | 6119 |
| Exon 8 | 6025-6148 | 98 | cacaacattggtgaggggggcg | 6139 | 6050 | 6249 |
| Exon 9 | 6241-6337 | 99 | tgggaccctggcccctgtctga | 6379 | 6290 | 6489 |
| Exon 10 | 6453-6586 | 100 | cgggacgagtaccggcgtctgg | 6481 | 6392 | 6591 |
| Exon 10 | 6453-6586 | 101 | cgtgctgcccgacgaagtgctg | 6561 | 6472 | 6671 |
| Exon 11 | 6661-6829 | 102 | ctggctccatccgcacggccga | 6670 | 6581 | 6780 |
| Exon 12 | 8930-9048 | 103 | ctccccgccagattctgtgctg | 8919 | 8830 | 9029 |
| Exon 12 | 8930-9048 | 104 | cagcacctacgccaaaggtaag | 9032 | 8943 | 9142 |
| Exon 13 | 12873-12942 | 105 | tactcccaggcagtacggggtg | 12750 | 12661 | 12860 |
| Exon 14 | 13029-13098 | 106 | tgtcatcatcacatctggggta | 13080 | 12991 | 13190 |
| Exon 15 | 13201-13302 | 107 | tgagatccctcccactgtcccg | 13173 | 13084 | 13283 |
| Exon 16 | 14844-14907 | 108 | cagcctgggcaacatggtgaca | 14703 | 14614 | 14813 |
| Exon 16 | 14844-14907 | 109 | tggtatgctgccagtggggctg | 14906 | 14817 | 15016 |
| Exon 17 | 15721-15842 | 110 | cctgacaggcagcctcagtggg | 15617 | 15528 | 15727 |
| Exon 17 | 15721-15842 | 111 | cagcatgtaggaatggggtgta | 15967 | 15878 | 16077 |
| Exon 17 | 15721-15842 | 112 | caggctgagaatgcagatataa | 16025 | 15936 | 16135 |
| Exon 18 | 17238-17330 | 113 | caccacatctcagatgagccag | 17112 | 17023 | 17222 |
| Exon 19 | 17417-17489 | 114 | ccatcctgctgtcagtggcccg | 17439 | 17350 | 17549 |
| Exon 19 | 17417-17489 | 115 | tggcccggggcaaagtgtccga | 17454 | 17365 | 17564 |
| Exon 20 | 17756-17826 | 116 | ccaactcagacacagcatcctg | 17661 | 17572 | 17771 |
| Exon 22 | 18220-18363 | 117 | cccactccccaccctcagcctg | 18436 | 18347 | 18546 |
| Exon 22 | 18220-18363 | 118 | ctgtcctcctgccctcagcaaa | 18459 | 18370 | 18569 |
| Exon 23 | 18851-18984 | 119 | ccaaattcattcaaacatcctg | 18730 | 18641 | 18840 |

TABLE XIII
XPE gene targets cleaved by I-CreI variants

| | | | |
|---|---|---|---|
| Exon closest | | | minimal repair |
| to the target | Target | target | matrix |

| | | | | | | |
|---|---|---|---|---|---|---|
| sequence | Exon position | SEQ ID NO | target sequence | position | start | end |

| | | | | | | |
|---|---|---|---|---|---|---|
| Exon 1 | 1-170 | 120 | caagcctcgacatgtcgtacaa | 99 | 10 | 209 |
| Exon 2 | 1387-1535 | 121 | caggacactttacttcggccga | 1384 | 1295 | 1494 |
| Exon 3 | 3004-3120 | 122 | taatattggtttccagggggag | 2988 | 2899 | 3098 |
| Exon 4 | 3494-3715 | 123 | tggccttttcaaggttattcca | 3577 | 3488 | 3687 |
| Exon 4 | 3494-3715 | 124 | ttgtctaccaggtactggatca | 3705 | 3616 | 3815 |
| Exon 5 | 6185-6299 | 125 | caggaccctcaggggcggcacg | 6182 | 6093 | 6292 |
| Exon 5 | 6185-6299 | 126 | ttccatggtgatcgcaggttgg | 6283 | 6194 | 6393 |
| Exon 6 | 7370-7467 | 127 | cccacccataggaatagtcgaa | 7123 | 7034 | 7233 |
| Exon 6 | 7370-7467 | 128 | tcttactctgtcgctcaggctg | 7742 | 7653 | 7852 |
| Exon 7 | 8941-9099 | 129 | taaaatggctgggtttggtaaa | 8842 | 8753 | 8952 |
| Exon 8 | 9984-10067 | 130 | ctgaattatggctgcagttggg | 9870 | 9781 | 9980 |
| Exon 8 | 9984-10067 | 131 | ccatcttgtgaaggtgagaaga | 10055 | 9966 | 10165 |
| Exon 9 | 10666-10782 | 132 | ccatctcttttgggtgggtctg | 10600 | 10511 | 10710 |
| Exon 9 | 10666-10782 | 133 | tggacctggagaggcaggggca | 10754 | 10665 | 10864 |
| Exon 10 | 11381-11483 | 134 | ttaaacatatctgaaagtataa | 11623 | 11534 | 11733 |
| Exon 11 | 16511-16586 | 135 | tctgaccctaatcgtgagactg | 16528 | 16439 | 16638 |
| Exon 12 | 16685-16793 | 136 | tcttctgtggcaacgtggctca | 16756 | 16667 | 16866 |
| Exon 13 | 18592-18770 | 137 | ttccccaccagctgtaggtttg | 18357 | 18268 | 18467 |
| Exon 13 | 18592-18770 | 138 | tgccatattcttctttgttcag | 18846 | 18757 | 18956 |
| Exon 14 | 18868-19031 | 139 | tggcctctggacggacatctcg | 18952 | 18863 | 19062 |
| Exon 15 | 19109-19216 | 140 | ttacacagaattcttagtccca | 19268 | 19179 | 19378 |
| Exon 16 | 19372-19579 | 141 | tgtcattctctctgtaggtctg | 19355 | 19266 | 19465 |
| Exon 16 | 19372-19579 | 142 | tgagatgggaagtatagtgcag | 19685 | 19596 | 19795 |
| Exon 17 | 20994-21089 | 143 | ccagaccaatgaaataagagta | 20803 | 20714 | 20913 |
| Exon 17 | 20994-21089 | 144 | tggcaccatcgatgagatccag | 21027 | 20938 | 21137 |
| Exon 18 | 21183-21294 | 145 | tctgctaccaggaagtgtccca | 21188 | 21099 | 21298 |
| Exon 19 | 22660-22783 | 146 | caggctctgtccagcagtgtaa | 22657 | 22568 | 22767 |
| Exon 19 | 22660-22783 | 147 | ctccctaatccaggctgttctg | 22821 | 22732 | 22931 |
| Exon 20 | 23118-23282 | 148 | tggtctttcagtattcggatgg | 23262 | 23173 | 23372 |
| Exon 20 | 23118-23282 | 149 | cagtattcggatggtaagtggg | 23270 | 23181 | 23380 |
| Exon 21 | 24001-24095 | 150 | tgtactctatggtggaatttaa | 24043 | 23954 | 24153 |
| Exon 21 | 24001-24095 | 151 | tagcacggtgaggcctggacca | 24089 | 24000 | 24199 |
| Exon 22 | 29043-29213 | 152 | ccagcccagtattaggggcaga | 29294 | 29205 | 29404 |
| Exon 23 | 29923-30032 | 153 | ccaaatgggctgcgttggcaga | 29740 | 29651 | 29850 |
| Exon 24 | 30327-30496 | 154 | tggtcttttccacctgggcgag | 30369 | 30280 | 30479 |
| Exon 24 | 30327-30496 | 155 | ttccacccccacacaaggctcg | 30444 | 30355 | 30554 |
| Exon 25 | 30719-30821 | 156 | caacctcctgctggacatgcag | 30750 | 30661 | 30860 |
| Exon 25 | 30719-30821 | 157 | tcgactcaataaagtcatcaaa | 30774 | 30685 | 30884 |
| Exon 26 | 32146-32269 | 158 | caagatgcaggaggtggtggca | 32239 | 32150 | 32349 |
| Exon 27 | 32859-33624 | 159 | ccatcttctcattgcagtatga | 32842 | 32753 | 32952 |
| Exon 27 | 32859-33624 | 160 | tatgacgatggcagcggtatga | 32859 | 32770 | 32969 |
| Exon 27 | 32859-33624 | 161 | cgacctcatcaaggttgtggag | 32900 | 32811 | 33010 |
| Exon 27 | 32859-33624 | 162 | ctaactcggatccattagccaa | 32925 | 32836 | 33035 |
| Exon 27 | 32859-33624 | 163 | tcggatccattagccaagggca | 32930 | 32841 | 33040 |
| Exon 27 | 32859-33624 | 164 | tgtcctctttttatttagattg | 33319 | 33230 | 33429 |
| Exon 27 | 32859-33624 | 165 | ctgactgccaagccatgggtag | 33458 | 33369 | 33568 |
| Exon 27 | 32859-33624 | 166 | ccaaataaagtagaatataaga | 33601 | 33512 | 33711 |

TABLE XIV
XPF gene targets cleaved by I-CreI variants

| | | | | |
|---|---|---|---|---|
| Exon | | | | |
| closest to | | Target | | minimal repair |
| the target | Exon | SEQ ID | target | matrix |

| | | | | | | |
|---|---|---|---|---|---|---|
| sequence | position | NO | target sequence | position | start | end |

| | | | | | | |
|---|---|---|---|---|---|---|
| Exon 1 | 1-207 | 167 | tgacacagagaaggatggcagg | 333 | 244 | 443 |
| Exon 2 | 1866-2046 | 168 | ctgccctgtattaaatagccta | 1820 | 1731 | 1930 |
| Exon 3 | 6396-6591 | 169 | tttgatactggtttttgtcatg | 6518 | 6429 | 6628 |
| Exon 3 | 6396-6591 | 170 | ctgtatctgtggccaaggtaaa | 6575 | 6486 | 6685 |
| Exon 4 | 7863-8070 | 171 | taacccatcgcttgaagtggaa | 8007 | 7918 | 8117 |
| Exon 5 | 10545-10725 | 172 | caagactaaatccttagttcag | 10589 | 10500 | 10699 |
| Exon 5 | 10545-10725 | 173 | ttgaataaagtgttaggtttta | 10765 | 10676 | 10875 |
| Exon 6 | 11992-12120 | 174 | tttaacttttcgtattaggttg | 11974 | 11885 | 12084 |
| Exon 6 | 11992-12120 | 175 | ttaacttffcgtattaggttgg | 11975 | 11886 | 12085 |
| Exon 6 | 11992-12120 | 176 | tgtaatgtatgttgaaagtata | 12265 | 12176 | 12375 |
| Exon 7 | 14027-14137 | 177 | tttgcttccaaaatctatcaaa | 14162 | 14073 | 14272 |
| Exon 7 | 14027-14137 | 178 | ttagctctttaaaagtagttca | 14199 | 14110 | 14309 |
| Exon 8 | 14981-15578 | 179 | tcatccatccgcttctgggttg | 15425 | 15336 | 15535 |
| Exon 8 | 14981-15578 | 180 | ctaacctttgttcggcagcttg | 15520 | 15431 | 15630 |
| Exon 8 | 14981-15578 | 181 | tttaatatccgttacgatgctg | 15663 | 15574 | 15773 |
| Exon 9 | 17601-17693 | 182 | tagtcctgctcaggaaggatag | 17762 | 17673 | 17872 |
| Exon 9 | 17601-17693 | 183 | cctgctcaggaaggatagggca | 17766 | 17677 | 17876 |
| Exon 10 | 24558-24670 | 184 | ttgtccctgaagaaagagaagg | 24575 | 24486 | 24685 |
| Exon 11 | 27449-28182 | 185 | cactccagaaatgtgcgtggag | 27585 | 27496 | 27695 |
| Exon 11 | 27449-28182 | 186 | cagcactggccattacagcaga | 27911 | 27822 | 28021 |
| Exon 11 | 27449-28182 | 187 | ctggccattacagcagattctg | 27916 | 27827 | 28026 |
| Exon 11 | 27449-28182 | 188 | cagaattagcagccctgtcaca | 28052 | 27963 | 28162 |

TABLE XV
XPG gene targets cleaved by I-CreI variants

| | | | | |
|---|---|---|---|---|
| Exon | | | | |
| closest to | | Target | | minimal repair |
| the target | Exon | SEQ ID | target | matrix |

| | | | | | | |
|---|---|---|---|---|---|---|
| sequence | position | NO | target sequence | position | start | end |

| | | | | | | |
|---|---|---|---|---|---|---|
| Exon 1 | 1-285 | 189 | cgggcctgtgggagcggggtca | 247 | 158 | 357 |
| Exon 2 | 6049-6224 | 190 | tggtatcttgcagacaagaaga | 6045 | 5956 | 6155 |
| Exon 3 | 7688-7803 | 191 | ccaacccatgtgcatgagtaca | 7780 | 7691 | 7890 |
| Exon 4 | 8219-8305 | 192 | caacccatgtgcatgagtacaa | 8133 | 8044 | 8243 |
| Exon 5 | 9983-10043 | 193 | tggaattatgcagtttattaag | 10066 | 9977 | 10176 |
| Exon 6 | 12206-12349 | 194 | tgttctcagaaagoagaggcca | 12290 | 12201 | 12400 |
| Exon 6 | 12206-12349 | 195 | ttaaatcatggccggcagctca | 12378 | 12289 | 12488 |
| Exon 7 | 15438-15645 | 196 | tttgcctgctgattagatttca | 15319 | 15230 | 15429 |
| Exon 7 | 15438-15645 | 197 | ttggacctcttagaagatgtaa | 15892 | 15803 | 16002 |
| Exon 8 | 15961-17034 | 198 | tatgacttccggaatgattctg | 16130 | 16041 | 16240 |
| Exon 8 | 15961-17034 | 199 | ttccctgcggtaagtggtacca | 16235 | 16146 | 16345 |
| Exon 8 | 15961-17034 | 200 | ccataccaggtaagcaggctgg | 16263 | 16174 | 16373 |
| Exon 8 | 15961-17034 | 201 | cgaccctcgtccgcgaagatga | 16483 | 16394 | 16593 |
| Exon 8 | 15961-17034 | 202 | ttaaattttctgattgggccta | 16622 | 16533 | 16732 |
| Exon 8 | 15961-17034 | 203 | tgtgctgaggtagctgggcctg | 16976 | 16887 | 17086 |
| Exon 9 | 19598-19842 | 204 | tcttactgtattacaggtctgg | 19576 | 19487 | 19686 |
| Exon 9 | 19598-19842 | 205 | caaaaccaagaaacgaatcttg | 19816 | 19727 | 19926 |
| Exon 9 | 19598-19842 | 206 | tttgccctaaaggaatatgcca | 19871 | 19782 | 19981 |
| Exon 10 | 20193-20312 | 207 | cggacctacgtctcagggggaa | 20296 | 20207 | 20406 |
| Exon 11 | 20563-20776 | 208 | ccatcttcatatccaaggtttg | 20589 | 20500 | 20699 |
| Exon 12 | 22044-22188 | 209 | taaaccactttagttaggaaga | 21979 | 21890 | 22089 |
| Exon 12 | 22044-22188 | 210 | cttactggctatttttatattg | 22206 | 22117 | 22316 |
| Exon 13 | 26129-26329 | 211 | ctgcatgaatctctgagggcag | 25965 | 25876 | 26075 |
| Exon 13 | 26129-26329 | 212 | tggtctctggaatgctagggag | 26539 | 26450 | 26649 |
| Exon 14 | 27190-27274 | 213 | tagtcctgcctggatgggccoa | 26921 | 26832 | 27031 |
| Exon 14 | 27190-27274 | 214 | tgggatttttttggaaatgttg | 27347 | 27258 | 27457 |
| Exon 15 | 29238-29926 | 215 | ccttccctccagcgttggccaa | 29269 | 29180 | 29379 |
| Exon 15 | 29238-29926 | 216 | ttggctgtgccttcataggtca | 29545 | 29456 | 29655 |

For example, for correcting some of the mutations in the XPC gene found in Xeroderma pigmentosum (XP), as indicated in FIG. 1A, the following combinations of variants/targeting constructs may be used:

ARG579TER (Exon 4; Premature Stop Codon):

variant: 28Q,33S,38R,40K,44Q,68Y,70S,75N,77Y (first monomer)/28T,33T,38Q,40R,44T,68E,70S,75R,77R (second monomer), and a targeting construct comprising at least positions 9887 to 10086 of the XPC gene, for efficient repair of the DNA double-strand break, and all sequences between the meganuclease cleavage site and the mutation site, for efficient repair of the mutation.

variant 30D,33R,38T,44N,68R,70S,75Q,77R (first monomer)/28R,33A,38Y,40Q,44D,68Y,70S,75S,77R (second monomer) and a targeting construct comprising at least positions 10173 to 10372 of the XPC gene, for efficient repair of the DNA double-strand break, and all sequences between the meganuclease cleavage site and the mutation site, for efficient repair of the mutation.

Exon 6: Substitution PRO218HIS:

variant: 28K,33N,38Q,40Q,44R,68Y,70S,75E,771 (first monomer)/28K,33T,38A,40Q,44K,68Q,70S,75N,77R (second monomer), and a targeting construct comprising at least positions 13051 to 13250 of the XPC gene, for efficient repair of the DNA double-strand break, and all sequences between the meganuclease cleavage site and the mutation site, for efficient repair of the mutation.

Exon 9: deletion DEL1132AA or insertion insVAL580:

*variant: 28K,30N,38Q,44Q,68R,70S,75R,77T,80K (first monomer)/28T,33T,38Q,40R,44T,68Y,70S,75R,77V (second monomer) and a targeting construct comprising at least positions 19580 to 19779 of the XPC gene, for efficient repair of the DNA double-strand break, and all sequences between the meganuclease cleavage site and the mutation site, for efficient repair of the mutation.

variant: 28K,30N,38Q,44A,68Y,70S,75Y,77K (first monomer)/28K,33R,38E,40R,44T,68R,70S,75Y,77T,133V (second monomer) and a targeting construct comprising at least positions 20303 to 20502 of the XPC gene, for efficient repair of the DNA double-strand break, and all sequences between the meganuclease cleavage site and the mutation site, for efficient repair of the mutation.

variants: 28K,33R,38Q,40A,44Q,68R,70N,75N (first monomer)/28K,33R,38A,40Q,44Q,68R,70S,75N,77K (second monomer) or 33H,75N (first monomer) and 33R,38A,40Q,44K,70N,75N (second monomer), and a targeting construct comprising at least positions 20349 to 20548 of the XPC gene, for efficient repair of the DNA double-strand break, and all sequences between the meganuclease cleavage site and the mutation site, for efficient repair of the mutation.

variant: 30N,33H,38Q,44K,68A,70S,75N,771 (first monomer)/28K,33N,38Q,40Q,44R,68Y,70S,75E,77V (second monomer), and a targeting construct comprising at least positions 20389 to 20588 of the XPC gene, for efficient repair of the DNA double-strand break, and all sequences between the meganuclease cleavage site and the mutation site, for efficient repair of the mutation.

Exon 14: Substitution LYS822GLN:

variant: 28Q,33S,38R,40K, 42T,44K,70S,75N,77Y (first monomer)/28R,33A,38Y,40Q,44A,68R,70S,75E,77R (second monomer), and a targeting construct comprising at least positions 30416 to 30615 of the XPC gene, for efficient repair of the DNA double-strand break, and all sequences between the meganuclease cleavage site and the mutation site, for efficient repair of the mutation.

Alternatively, for restoring a functional gene (FIG. 1C), cleavage of the gene occurs upstream of a mutation, for example at position 9119 (target SEQ ID NO: 24). Preferably said mutation is the first known mutation in the sequence of the gene, so that all the downstream mutations of the gene can be corrected simultaneously. The targeting construct comprises the exons downstream of the cleavage site fused in frame (as in the cDNA) and with a polyadenylation site to stop transcription in 3′. The sequence to be introduced (exon knock-in construct) is flanked by introns or exons sequences surrounding the cleavage site, so as to allow the transcription of the engineered gene (exon knock-in gene) into a mRNA able to code for a functional protein (FIG. 1C). For example, when cleavage occurs in an exon, the exon knock-in construct is flanked by sequences upstream and downstream of the cleavage site, from a minimal repair matrix as defined above.

The subject-matter of the present invention is also a composition characterized in that it comprises at least one variant, one single-chain chimeric endonuclease and/or at least one expression vector encoding said variant/single-chain molecule, as defined above.

In a preferred embodiment of said composition, it comprises a targeting DNA construct comprising a sequence which repairs a mutation in the XP gene, flanked by sequences sharing homologies with the genomic DNA cleavage site of said variant, as defined above. The sequence which repairs the mutation is either a fragment of the gene with the correct sequence or an exon knock-in construct, as defined above.

Preferably, said targeting DNA construct is either included in a recombinant vector or it is included in an expression vector comprising the polynucleotide(s) encoding the variant/single-chain chimeric endonuclease according to the invention.

In the case where two vectors may be used, the subject-matter of the present invention is also products containing an I-CreI variant or single-chain chimeric meganuclease expression vector as defined above and a vector which includes a targeting construct as defined above, as a combined preparation for simultaneous, separate or sequential use in Xeroderma pigmentosum.

The subject-matter of the present invention is also the use of at least one meganuclease variant/single-chain chimeric meganuclease and/or one expression vector, as defined above, for the preparation of a medicament for preventing, improving or curing Xeroderma pigmentosum in an individual in need thereof, said medicament being administrated by any means to said individual.

In this case, the use of the meganuclease (variant/single-chain derivative) comprises at least the step of (a) inducing in somatic tissue(s) of the individual a double stranded cleavage at a site of interest comprising at least one recognition and cleavage site of said meganuclease by contacting said cleavage site with said meganuclease, and (b) introducing into the individual a targeting DNA, wherein said targeting DNA comprises (1) DNA sharing homologies to the region surrounding the cleavage site and (2) DNA which repairs the site of interest upon recombination between the targeting DNA and the chromosomal DNA. The targeting DNA is introduced into the individual under conditions appropriate for introduction of the targeting DNA into the site of interest.

According to the present invention, said double-stranded cleavage is induced, either in toto by administration of said meganuclease to an individual, or ex vivo by introduction of said meganuclease into somatic cells (skin cells) removed from an individual and returned into the individual after modification.

The subject-matter of the present invention is also a method for preventing, improving or curing Xeroderma pigmentosum in an individual in need thereof, said method comprising at least the step of administering to said individual a composition as defined above, by any means.

The meganuclease (variant/single-chain derivative) can be used either as a polypeptide or as a polynucleotide construct encoding said polypeptide. It is introduced into somatic cells of an individual, by any convenient mean well-known to those in the art, which is appropriate for the particular cell type, alone or in association with either at least an appropriate vehicle or carrier and/or with the targeting DNA.

According to an advantageous embodiment of the uses according to the invention, the meganuclease (polypeptide) is associated with:
liposomes, polyethyleneimine (PEI); in such a case said association is administered and therefore introduced into somatic target cells.
membrane translocating peptides (Bonetta, 2002, The Scientist, 16, 38; Ford et al, Gene Ther, 2001, 8, 1-4; Wadia & Dowdy, 2002, Curr Opin Biotechnol, 13, 52-56); in such a case, the sequence of the variant/single-chain derivative is fused with the sequence of a membrane translocating peptide (fusion protein).

According to another advantageous embodiment of the uses according to the invention, the meganuclease (polynucleotide encoding said meganuclease) and/or the targeting DNA is inserted in a vector. Vectors comprising targeting DNA and/or nucleic acid encoding a meganuclease can be introduced into a cell by a variety of methods (e.g., injection, direct uptake, projectile bombardment, liposomes). Meganucleases can be stably or transiently expressed into cells using expression vectors. Techniques of expression in eukaryotic cells are well known to those in the art. (See Current Protocols in Human Genetics: Chapter 12 “Vectors For Gene Therapy” & Chapter 13 “Delivery Systems for Gene Therapy”). Optionally, it may be preferable to incorporate a nuclear localization signal into the recombinant protein to be sure that it is expressed within the nucleus.

Once in a cell, the meganuclease and if present, the vector comprising targeting DNA and/or nucleic acid encoding a meganuclease are imported or translocated by the cell from the cytoplasm to the site of action in the nucleus.

For purposes of therapy, the meganucleases and a pharmaceutically acceptable excipient are administered in a therapeutically effective amount. Such a combination is said to be administered in a “therapeutically effective amount” if the amount administered is physiologically significant. An agent is physiologically significant if its presence results in a detectable change in the physiology of the recipient. In the present context, an agent is physiologically significant if its presence results in a decrease in the severity of one or more symptoms of the targeted disease and in a genome correction of the lesion or abnormality.

In one embodiment of the uses according to the present invention, the meganuclease is substantially non-immunogenic, i.e., engender little or no adverse immunological response. A variety of methods for ameliorating or eliminating deleterious immunological reactions of this sort can be used in accordance with the invention. In a preferred embodiment, the meganuclease is substantially free of N-formyl methionine. Another way to avoid unwanted immunological reactions is to conjugate meganucleases to polyethylene glycol (“PEG”) or polypropylene glycol (“PPG”) (preferably of 500 to 20,000 daltons average molecular weight (MW)). Conjugation with PEG or PPG, as described by Davis et al., (U.S. Pat. No. 4,179,337) for example, can provide non-immunogenic, physiologically active, water soluble endonuclease conjugates with anti-viral activity. Similar methods also using a polyethylene-poly-propylene glycol copolymer are described in Saifer et al. (U.S. Pat. No. 5,006,333).

The invention also concerns a prokaryotic or eukaryotic host cell which is modified by a polynucleotide or a vector as defined above, preferably an expression vector.

The invention also concerns a non-human transgenic animal or a transgenic plant, characterized in that all or part of their cells are modified by a polynucleotide or a vector as defined above.

As used herein, a cell refers to a prokaryotic cell, such as a bacterial cell, or an eukaryotic cell, such as an animal, plant or yeast cell.

The subject-matter of the present invention is further the use of a meganuclease (variant or single-chain derivative) as defined above, one or two polynucleotide(s), preferably included in expression vector(s), for genome engineering (animal models generation: knock-in or knock-out), for non-therapeutic purposes.

According to an advantageous embodiment of said use, it is for inducing a double-strand break in the gene of interest, thereby inducing a DNA recombination event, a DNA loss or cell death.

According to the invention, said double-strand break is for: repairing a specific sequence, modifying a specific sequence, restoring a functional gene in place of a mutated one, attenuating or activating an endogenous gene of interest, introducing a mutation into a site of interest, introducing an exogenous gene or a part thereof, inactivating or deleting an endogenous gene or a part thereof, translocating a chromosomal aim, or leaving the DNA unrepaired and degraded.

According to another advantageous embodiment of said use, said variant, polynucleotide(s), vector are associated with a targeting DNA construct as defined above.

In a first embodiment of the use of the meganuclease (variant/single-chain derivative) according to the present invention, it comprises at least the following steps: 1) introducing a double-strand break at the genomic locus comprising at least one recognition and cleavage site of said meganuclease by contacting said cleavage site with said meganuclease; 2) providing a targeting DNA construct comprising the sequence to be introduced flanked by sequences sharing homologies to the targeted locus. Said meganuclease variant can be provided directly to the cell or through an expression vector comprising the polynucleotide sequence encoding said meganuclease and suitable for its expression in the used cell. This strategy is used to introduce a DNA sequence at the target site, for example to generate knock-in or knock-out animal models or cell lines that can be used for drug testing.

The subject-matter of the present invention is also the use of at least one meganuclease variant, as defined above, as a scaffold for making other meganucleases. For example a third round of mutagenesis and selection/screening can be performed on said variants, for the purpose of making novel, third generation homing endonucleases.

The different uses of the I-CreI variant and the methods of using said I-CreI variant according to the present invention include also the use of the single-chain chimeric meganuclease derived from said variant, the polynucleotide(s), vector, cell, transgenic plant or non-human transgenic mammal encoding said variant or single-chain chimeric endonuclease, as defined above.

The I-CreI variant according to the invention may be obtained by a method for engineering I-CreI variants able to cleave a genomic DNA target sequence from a gene of interest, for example a mammalian gene, comprising at least the steps of:

(a) constructing a first series of I-CreI variants having at least one substitution in a first functional subdomain of the LAGLIDADG core domain situated from positions 26 to 40 of I-CreI, preferably from positions 28 to 40 of I-CreI,

(b) constructing a second series of I-CreI variants having at least one substitution in a second functional subdomain of the LAGLIDADG core domain situated from positions 44 to 77 of I-CreI, preferably from positions 44 to 70 of I-CreI,

(c) selecting and/or screening the variants from the first series of step (a) which are able to cleave a mutant I-CreI site wherein (i) the nucleotide triplet in positions −10 to −8 of the I-CreI site has been replaced with the nucleotide triplet which is present in positions −10 to −8 of said genomic target and (ii) the nucleotide triplet in positions +8 to +10 has been replaced with the reverse complementary sequence of the nucleotide triplet which is present in positions −10 to −8 of said genomic target

(d) selecting and/or screening the variants from the second series of step (b) which are able to cleave a mutant I-CreI site wherein (i) the nucleotide triplet in positions −5 to −3 of the I-CreI site has been replaced with the nucleotide triplet which is present in positions −5 to −3 of said genomic target and (ii) the nucleotide triplet in positions +3 to +5 has been replaced with the reverse complementary sequence of the nucleotide triplet which is present in positions −5 to −3 of said genomic target (e) selecting and/or screening the variants from the first series of step (a) which are able to cleave a mutant I-CreI site wherein (i) the nucleotide triplet in positions +8 to +10 of the I-CreI site has been replaced with the nucleotide triplet which is present in positions +8 to +10 of said genomic target and (ii) the nucleotide triplet in positions −10 to −8 has been replaced with the reverse complementary sequence of the nucleotide triplet which is present in positions +8 to +10 of said genomic target

(f) selecting and/or screening the variants from the second series of step (b) which are able to cleave a mutant I-CreI site wherein (i) the nucleotide triplet in positions +3 to +5 of the I-CreI site has been replaced with the nucleotide triplet which is present in positions +3 to +5 of said genomic target and (ii) the nucleotide triplet in positions −5 to −3 has been replaced with the reverse complementary sequence of the nucleotide triplet which is present in positions +3 to +5 of said genomic target

(g) combining in a single variant, the mutation(s) in positions 28 to 40 and 44 to 70 of two variants from step (c) and step (d), to obtain a novel homodimeric I-CreI variant which cleaves a sequence wherein (i) the nucleotide triplet in positions −10 to −8 is identical to the nucleotide triplet which is present in positions −10 to −8 of said genomic target, (ii) the nucleotide triplet in positions +8 to +10 is identical to the reverse complementary sequence of the nucleotide triplet which is present in positions −10 to −8 of said genomic target, (iii) the nucleotide triplet in positions −5 to −3 is identical to the nucleotide triplet which is present in positions −5 to −3 of said genomic target and (iv) the nucleotide triplet in positions +3 to +5 is identi-cal to the reverse complementary sequence of the nucleotide triplet which is present in positions −5 to −3 of said genomic target

(h) combining in a single variant, the mutation(s) in positions 28 to 40 and 44 to 70 of two variants from step (e) and step (f), to obtain a novel homodimeric I-CreI variant which cleaves a sequence wherein (i) the nucleotide triplet in positions +3 to +5 is identical to the nucleotide triplet which is present in positions +3 to +5 of said genomic target, (ii) the nucleotide triplet in positions −5 to −3 is identical to the reverse complementary sequence of the nucleotide triplet which is present in positions +3 to +5 of said genomic target, (iii) the nucleotide triplet in posi-tions+8 to +10 of the I-CreI site has been replaced with the nucleotide triplet which is present in positions +8 to +10 of said genomic target and (iv) the nucleotide triplet in positions −10 to −8 is identical to the reverse complementary sequence of the nucleo-tide triplet in positions +8 to +10 of said genomic target.

(i) combining the variants obtained in steps (g) and (h) to form heterodimers.

(j) selecting and/or screening the heterodimers from step (i) which are able to cleave said genomic DNA target situated in a mammalian gene.

Steps (a), (b), (g), (h) and (i) may further comprise the introduction of additional mutations in order to improve the binding and/or cleavage properties of the mutants. Additional mutations may be introduced at other positions contacting the DNA target sequence or interacting directly or indirectly with said DNA target. This additional step may be performed by generating a library of variants as described in the International PCT Application WO 2004/067736.

The method for engineering I-CreI variants of the invention advantageously comprise the introduction of random mutations on the whole variant or in a part of the variant, in particular the C-terminal half of the variant (positions 80 to 163) to improve the binding and/or cleavage properties of the mutants towards the DNA target from the gene of interest. The mutagenesis may be performed by generating random mutagenesis libraries on a pool of variants, according to standard mutagenesis methods which are well-known in the art and commercially available. Preferably, the mutagenesis is performed on the entire sequence of one monomer of the heterodimer formed in step (i) or obtained in step (j), advantageously on a pool of monomers, preferably on both monomers of the heterodimer of step (i) or (j).

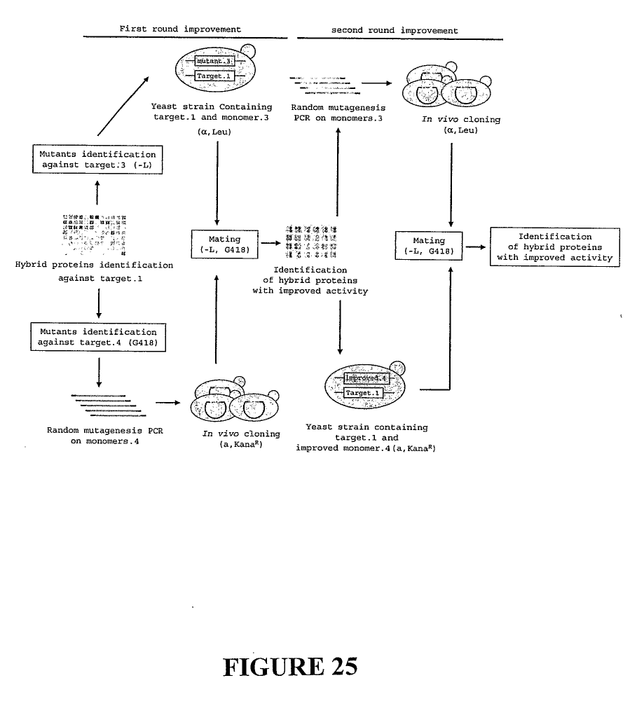

Preferably, two rounds of selection/screening are performed according to the process illustrated by FIG. 25. In the first round, one of the monomers of the heterodimer is mutagenised (monomer.4 in FIG. 25), co-expressed with the other monomer (monomer.3 in FIG. 25) to form heterodimers, and the improved monomers.4 are selected against the target from the gene of interest. In the second round, the other monomer (monomer.3) is mutagenised, co-expressed with the improved monomers.4 to form heterodimers, and selected against the target from the gene of interest to obtain meganucleases with improved activity.

The combination of mutations in steps (g) and (h) may be performed by amplifying overlapping fragments comprising each of the two subdomains, according to well-known overlapping PCR techniques.

The combination of the variants in step (i) is performed by co-expressing one variant from step (g) with one variant from step (h), so as to allow the formation of heterodimers. For example, host cells may be modified by one or two recombinant expression vector(s) encoding said variant(s). The cells are then cultured under conditions allowing the expression of the variant(s), so that heterodimers are formed in the host cells.

The selection and/or screening in steps (c), (d), (e), (f) and/or G) may be performed by using a cleavage assay in vitro or in vivo, as described in the International PCT Application WO 2004/067736 or in Arnould et al., J. Mol. Biol., 2006, 355(3): 443-58.

According to another advantageous embodiment of said method, steps (c), (d), (e), (f) and/or (j) are performed in vivo, under conditions where the double-strand break in the mutated DNA target sequence which is generated by said variant leads to the activation of a positive selection marker or a reporter gene, or the inactivation of a negative selection marker or a reporter gene, by recombination-mediated repair of said DNA double-strand break.

The polynucleotide sequence(s) encoding the variant as defined in the present invention may be prepared by any method known by the man skilled in the art. For example, they are amplified from a cDNA template, by polymerase chain reaction with specific primers. Preferably the codons of said cDNA are chosen to favour the expression of said protein in the desired expression system.

The recombinant vector comprising said polynucleotides may be obtained and introduced in a host cell by the well-known recombinant DNA and genetic engineering techniques.

The variant of the invention is produced by expressing the poly-peptide(s) as defined above; preferably said polypeptide(s) are expressed or co-expressed in a host cell modified by one or two expression vector(s), under conditions suitable for the expression or co-expression of the polypeptides, and the variant is recovered from the host cell culture.

Single-chain chimeric meganucleases able to cleave a DNA target from the gene of interest are derived from the variants according to the invention by methods well-known in the art (Epinat et al., Nucleic Acids Res., 2003, 31, 2952-62; Chevalier et al., Mol. Cell., 2002, 10, 895-905; Steuer et al., Chembiochem., 2004, 5, 206-13; International PCT Applications WO 03/078619 and WO 2004/031346). Any of such methods, may be applied for constructing single-chain chimeric meganucleases derived from the variants as defined in the present invention.

In addition to the preceding features, the invention further comprises other features which will emerge from the description which follows, which refers to examples illustrating the I-CreI meganuclease variants and their uses according to the invention, as well as to the appended drawings in which:

FIG. 1 represents the human XPC gene, and two different strate-gies for restoring a functional gene by meganuclease-induced recombination. A. The XPC gene CDS junctions are indicated; the mutations found in the XP-C complementation group are featured by an arrow. The Xa.1 sequence (position 9119, SEQ ID NO: 24) is found in an intronic sequence. The Xc.1 sequence (position 20438, SEQ ID NO: 12) is found in Exon 9. B. Gene correction. A mutation occurs within a known gene. Upon cleavage by a meganuclease and recombination with a repair matrix the deleterious mutation is corrected. C. Exonic sequences knock-in. A mutation occurs within a known gene. The mutated mRNA transcript is featured below the gene. In the repair matrix, exons located downstream of the cleavage site are fused in frame (as in a cDNA), with a polyadenylation site to stop transcription in 3′. Introns and exons sequences can be used as homologous regions. Exonic sequences knock-in results into an engineered gene, transcribed into a mRNA able to code for a functional protein.

FIG. 2 illustrates the principle of the invention. A: Structure of 1-CreI bound to its target. Experimental data have shown that two independent subdomains (squares) could be identified in the DNA binding domain; each subdomain of the core domain binds a different half of the DNA target. B. One would like to identify smaller independent subdomains (squares), each binding a distinct part of a half DNA target. However, there is no structural or experimental data in favour of this hypothesis.

FIG. 3 represents the map of the base specific interactions of 1-CreI with its DNA target C1221 (SEQ ID NO: 25) (Chevalier and Stoddard, Nucleic Acids Res., 2001, 29, 3757-74; Chevalier et al. J. Mol. Biol., 2003, 329, 253-69). The inventors have identified novel I-CreI derived endonucleases able to bind DNA targets modified in regions −−10 to −8 and +8 to +10, or −5 to −3 and +3 to +5. These DNA regions are indicated in grey boxes.

FIG. 4 illustrates the rationale of the combinatorial approach. A. Given the separability of the two DNA binding subdomain (top left), one can combine different I-CreI monomers binding different sequences derived from the I-CreI target sequence (top right and bottom left) to obtain heterodimers or single chain fusion molecules cleaving non-palindromic chimeric targets (bottom right). B. The identifi-cation of smaller independent subunit, i.e., subunit within a single monomer or αββαββα fold (top right and bottom left) would allow for the design of novel chimeric molecules (bottom right), by combination of mutations within the same monomer. Such molecules would cleave palindromic chimeric targets (bottom right). C. The combination of the two former steps would allow a larger combinatorial approach, involving four different subdomains (top right, middle left and right, bottom left) that could be combined in new molecules (bottom right). Thus, the identification of a small number of new cleavers for each subdomain would allow for the design of a very large number of novel endonucleases.

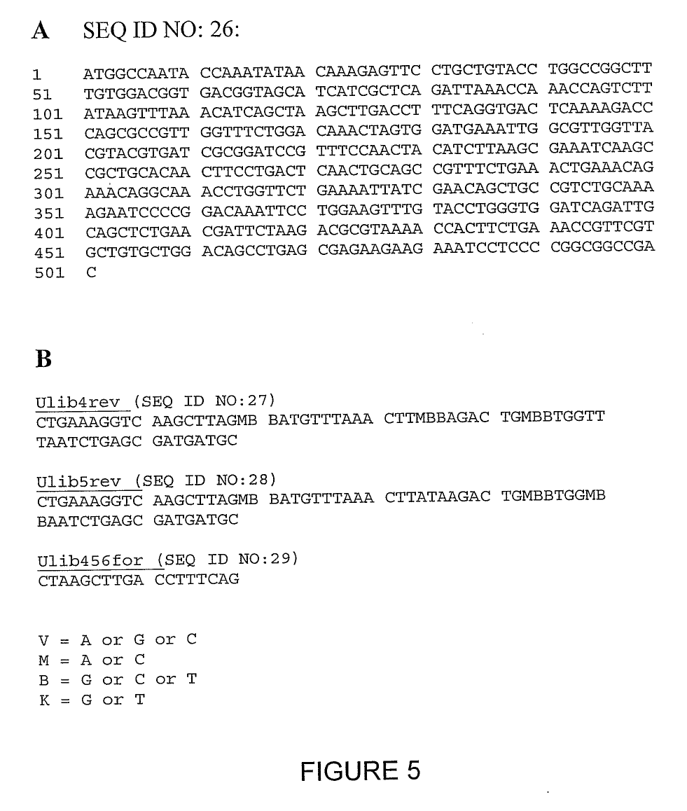

FIG. 5 represents the sequences of the cDNA encoding I-CreI N75 scaffold protein and degenerated primers used for the Ulib4 and Ulib5 libraries construction. A. The scaffold (SEQ ID NO: 26) encodes an I-CreI ORF (SEQ ID NO: 218) including the insertion of an alanine codon in position 2, the A42T, D75N, W110E and R111Q codons substitutions and three additional codons (AAD) at the 3′ end. B. Primers (SEQ ID NO: 27, 28, 29),

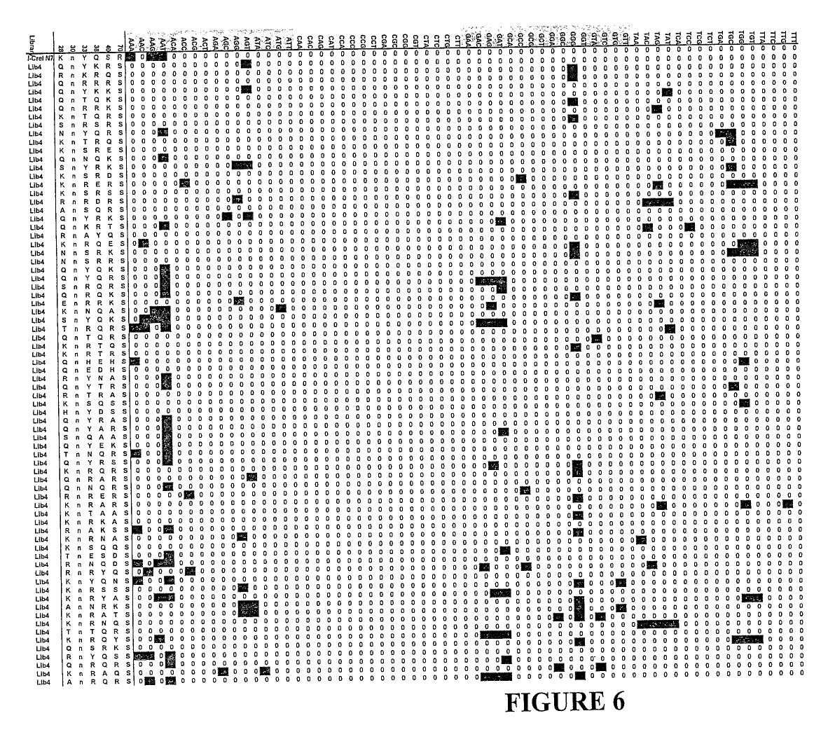

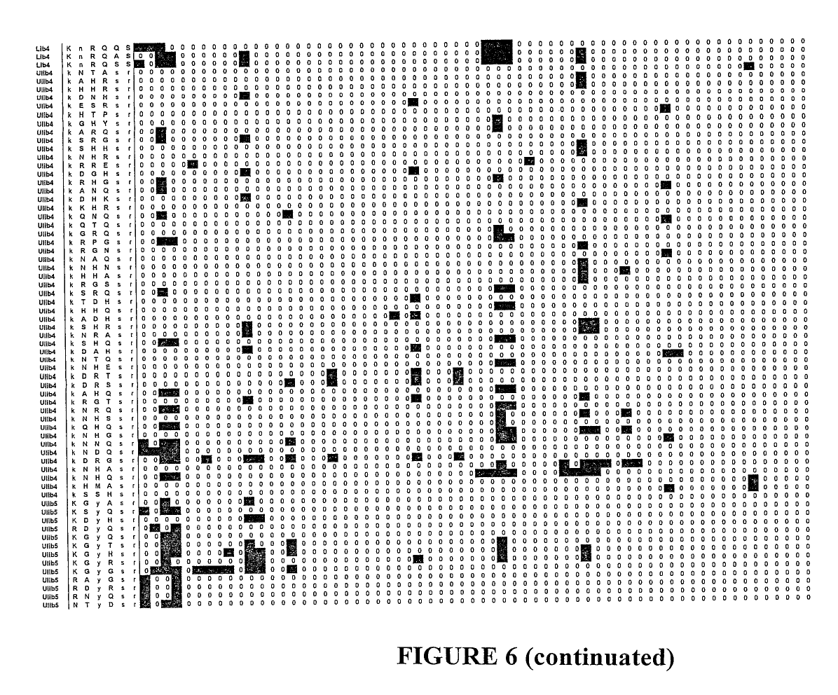

FIG. 6 represents the cleavage patterns of the I-CreI variants in positions 28, 30, 33, 38 and/or 40. For each of the 141 I-CreI variants obtained after screening, and defined by residues in position 28, 30, 33, 38, 40, 70 and 75, cleavage was monitored in yeast with the 64 targets derived from the C1221 palindromic target cleaved by I-CreI, by substitution of the nucleotides in positions ±8 to 10.Targets are designated by three letters, corresponding to the nucleotides in position −10, −9 and −8. For example GGG corresponds to the tcgggacgtcgtacgacgtcccga target (SEQ ID NO: 30). Values correspond to the intensity of the cleavage, evaluated by an appropriate software after scanning of the filter. For each protein, observed cleavage (black box) or non observed cleavage (0) is shown for each one of the 64 targets. All the variants are mutated in position 75: D75N.

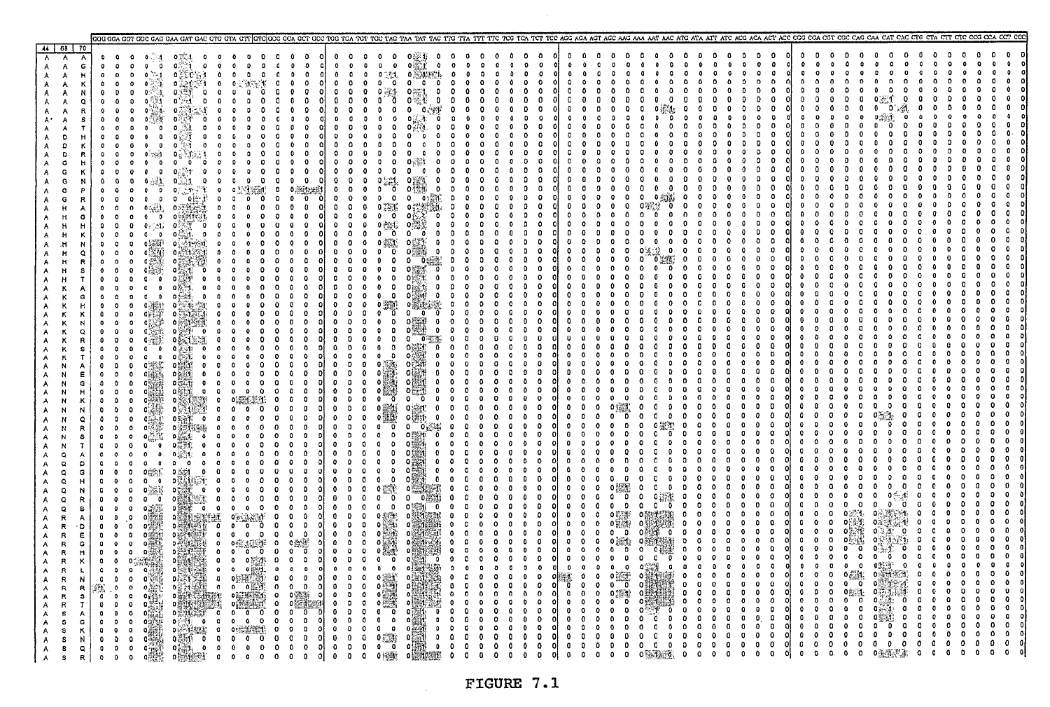

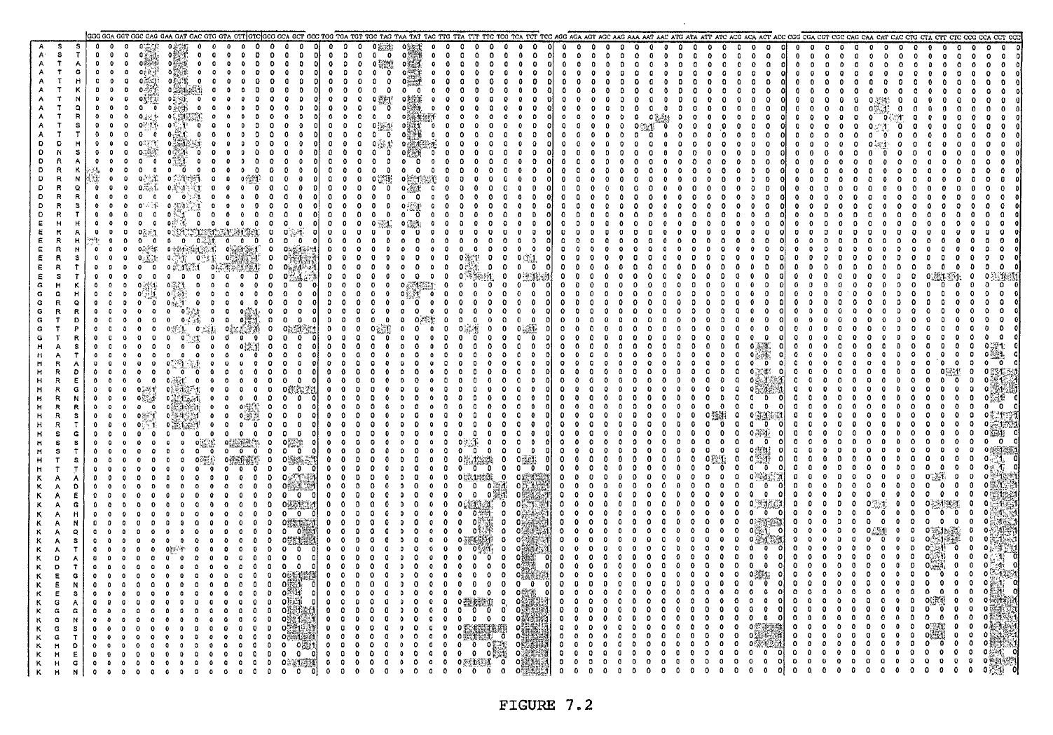

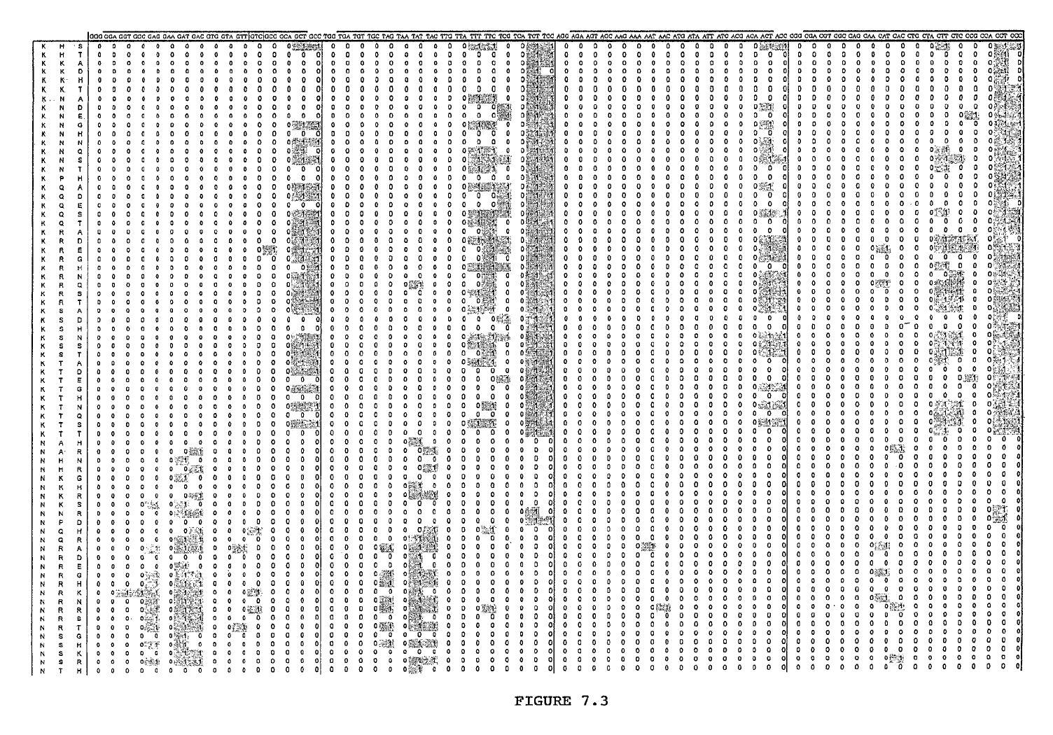

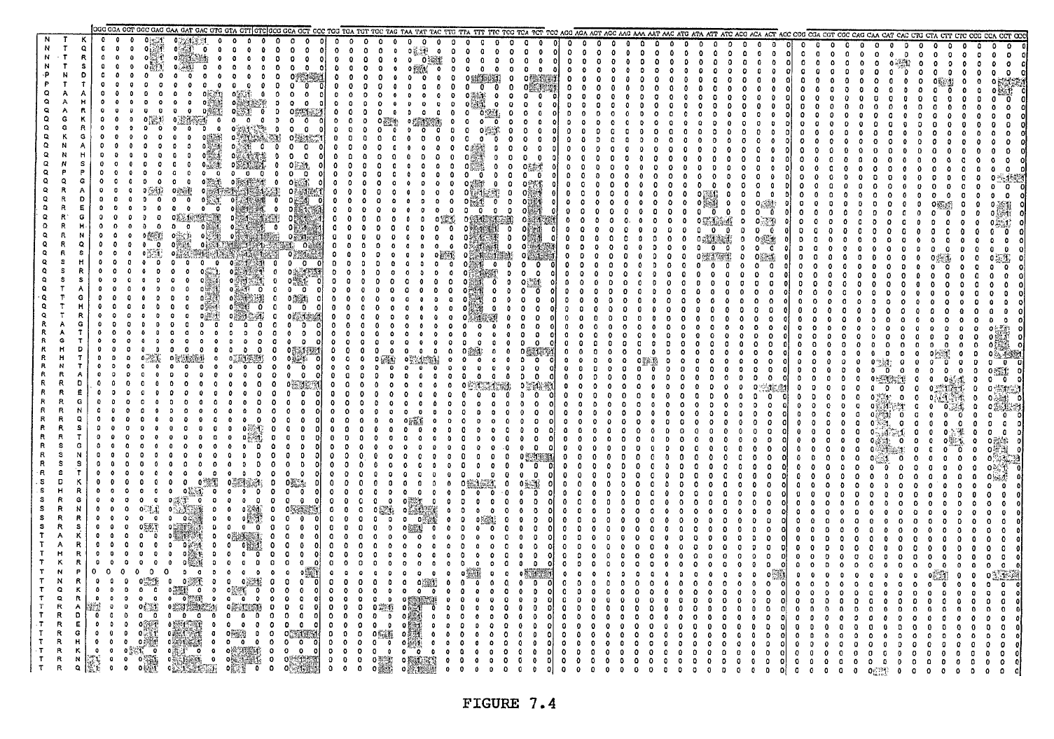

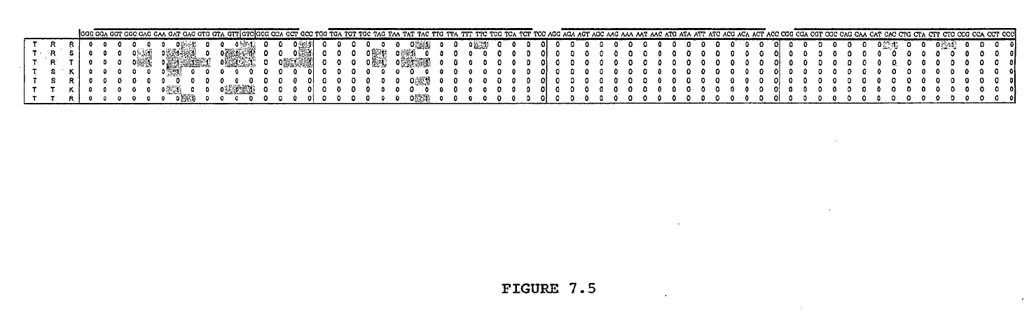

FIG. 7 represents the cleavage patterns of the I-CreI variants in position 44, 68 and/or 70. For each of the 292 I-CreI variants obtained after screening, and defined by residues in position 44, 68 and 70 (first three columns) cleavage was monitored in yeast with the 64 targets derived from the C1221 palindromic target cleaved by I-CreI, by substitution of the nucleotides in positions ±3 to 5. Targets are designated by three letters, corresponding to the nucleotides in position −5, −4 and −3. For each protein, observed cleavage (1) or non observed cleavage (0) is shown for each one of the 64 targets. All the variants are mutated in position 75: D75N.

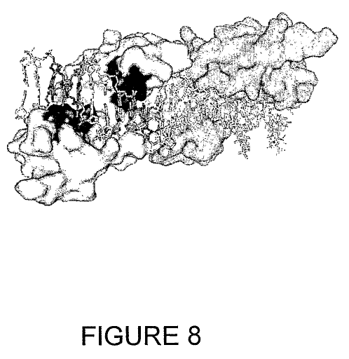

FIG. 8 represents the localisation of the mutations in the protein and DNA target, on a I-CreI homodimer bound to its target. The two set of mutations (residues 44, 68 and 70; residues 28, 30, 33, 38 and 40 are shown in black on the monomer on the left. The two sets of mutations are clearly distinct spatially. However, there is no structural evidence for distinct subdomains. Cognate regions in the DNA target site (region −5 to −3; region −10 to −8) are shown in grey on one half site.

FIG. 9 represents the Xa series of targets and close derivatives. C1221 (SEQ ID NO: 25) is one of the I-CreI palindromic target sequences. 10TGC_P, 10AGG_P, 5CCT_P and 5TTT (SEQ ID NO: 31, 32, 33, 34) are close derivatives found to be cleaved by I-CreI mutants. They differ from C1221 by the boxed motives. C1221, 10TGC_P, 10AGG_P, 5CCT_P and 5TTT were first described as 24 bp sequences, but structural data suggest that only the 22 bp are relevant for protein/DNA interaction. However, positions ±12 are indicated in parenthesis.Xa.1 (SEQ ID NO:24) is the DNA sequence located in the human XPC gene at position 9119. In the Xa.2 target (SEQ ID NO:35), the TTGA sequence in the middle of the target is replaced with GTAC, the bases found in C1221. Xa.3 (SEQ ID NO:36) is the palindromic sequence derived from the left part of Xa.2, and Xa.4 (SEQ ID NO:37) is the palindromic sequence derived from the right part of Xa.2. The boxed motives from 10TGC_P, 10AGG_P, 5CCT_P and 5TTT are found in the Xa series of targets.

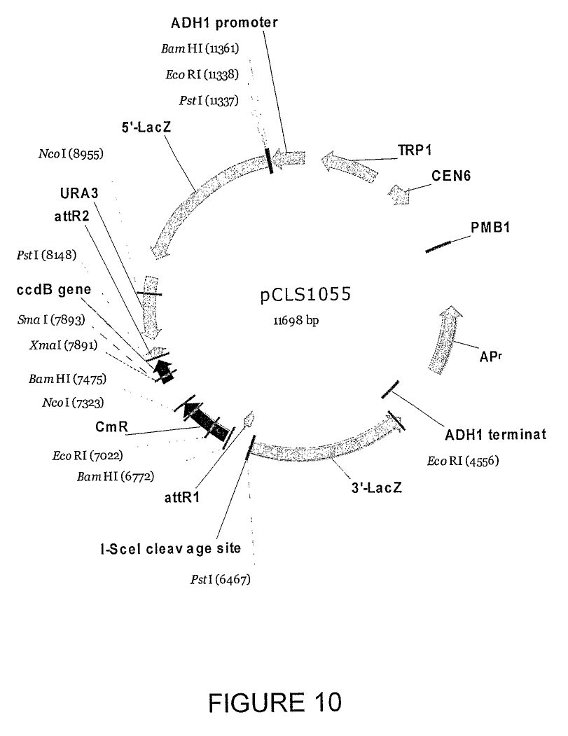

FIG. 10 represents the pCLS1055 plasmid vector map.

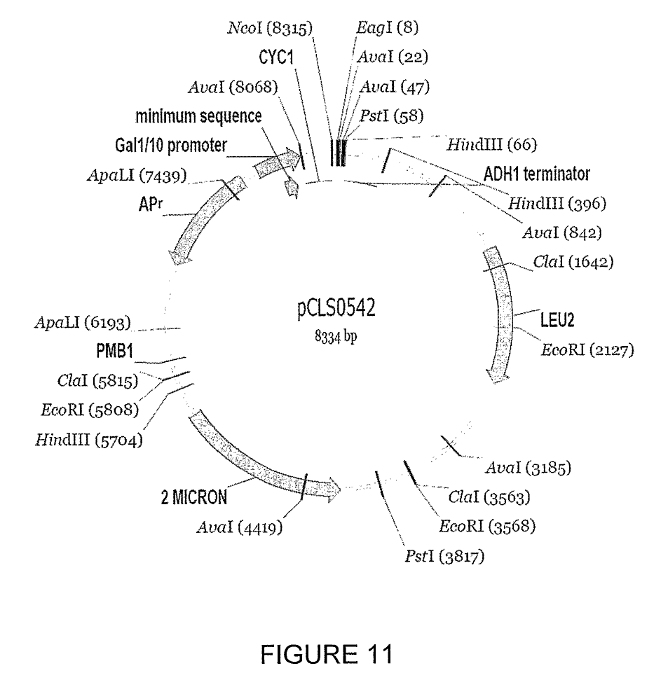

FIG. 11 represents the pCLS10542 plasmid vector map.

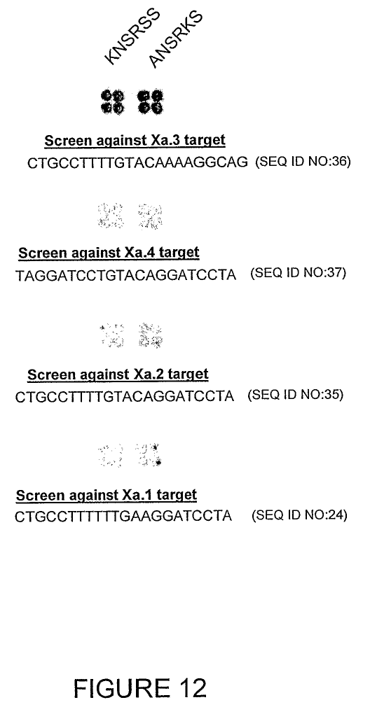

FIG. 12 illustrates the cleavage of the Xa.3 target. The figure displays secondary screening of the I-CreI K28, N30, S33, R38, S40, S70 N75 (KNSRSSN) and I-CreI A28, N30, S33, R38, K40, S70 N75 (ANSRKSN) mutants with Xa.1, Xa.2, Xa.3 and Xa.4 targets (SEQ ID NO:24, 35, 36, 37).

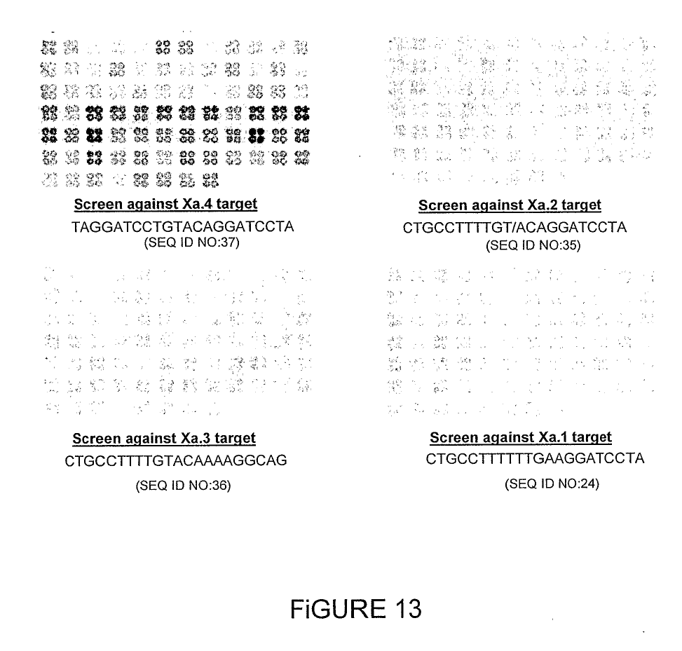

FIG. 13 illustrates the cleavage of the Xa.4 target. The figure displays secondary screening of a series of combinatorial mutants among those described in Table XVI with Xa.1, Xa.2, Xa.3 and Xa.4 targets (SEQ ID NO:24, 35, 36, 37).

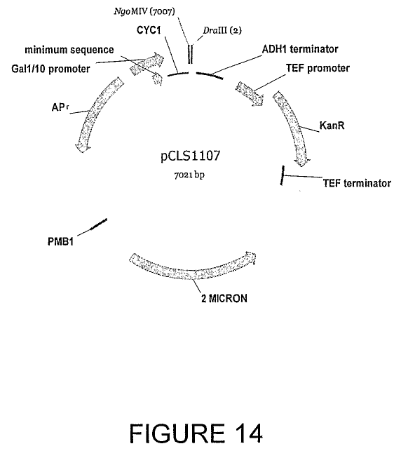

FIG. 14 represents the pCLS1107 vector map.

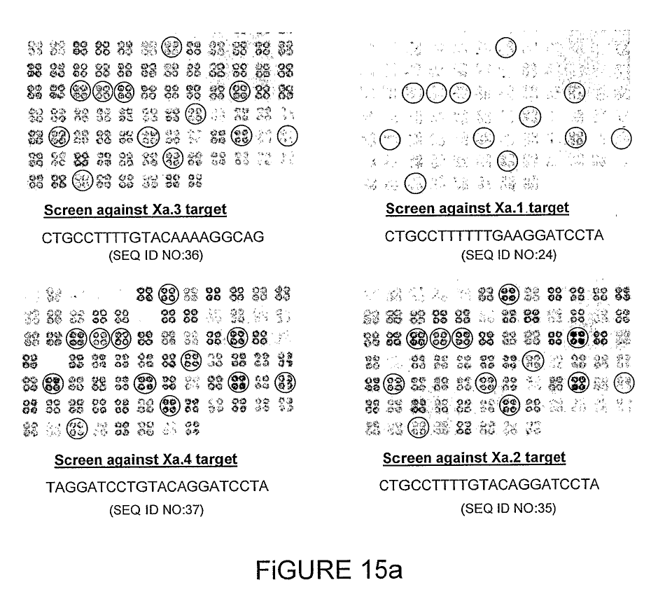

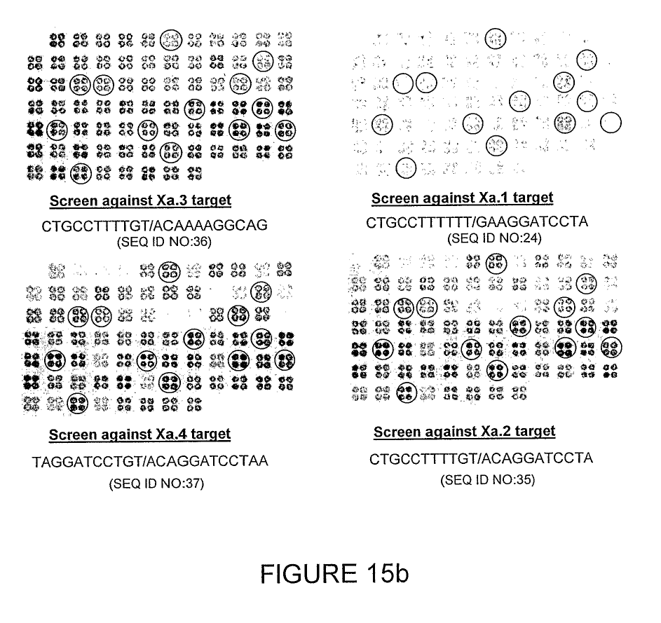

FIG. 15 illustrates the cleavage of the Xa.1 and Xa.2 targets. A series of I-CreI N75 mutants cutting Xa.4 are co-expressed with either KNSRSS (a) or ANSRKS (b). Cleavage is tested with the Xa.1, Xa.2, Xa.3 and Xa.4 targets (SEQ ID NO:24, 35, 36, 37). Mutants cleaving Xa.1 are circled.

FIGS. 16 to 22 illustrate the DNA target sequences found in each exon of the human XP genes (XPA to XPG gene) and the corresponding I-CreI variant which is able to cleave said DNA target. The exons closest to the target sequences, and the exons junctions are indicated (columns 1 and 2), the sequence of the DNA target is presented (column 3), with its position (column 4). The minimum repair matrix for repairing the cleavage at the target site is indicated by its first nucleotide (start, column 7) and last nucleotide (end, column 8). The sequence of each variant is defined by its amino acid residues at the indicated positions. For example, the first heterodimeric variant of FIG. 16 consists of a first monomer having K, S, R, D, K, R, G and N in positions 28, 33, 38, 40, 44, 68, 70 and 75, respectively and a second monomer having R, D, R, K, A, S, N and I in positions 28, 30, 38, 44, 68, 70, 75 and 77, respectively. The positions are indicated by reference to I-CreI sequence SWISSPROT P05725 or pdb accession code 1 g9y; I-CreI has I, Q, K, N, S, Y, Q, S, A, Q, R, R, D, I, E and A, in positions 24, 26, 28, 30, 32, 33, 38, 40, 42, 44, 68, 70, 75, 77, 80 and 133, respectively. The positions which are not indicated may be mutated or not mutated. In the latter case, the positions which are not indicated correspond to the wild-type I-CreI sequence.

FIG. 23 represents three targets, derivated from C1221: Xa.1, Xb.1 and Xc. 1 (SEQ ID NO: 24, 8, 12), identified in the human XPC gene. Each initial target was transformed in a Xx.2 target (SEQ ID NO: 35, 219, 222) more favourable for I-CreI cleavage, then in two palindromics targets Xx.3 (SEQ ID NO: 36, 220, 223) and Xx.4 (SEQ ID NO: 37, 221, 224). Stars represent potential targets found in the gene. Black squares or vertical lines represent the XPC exons.

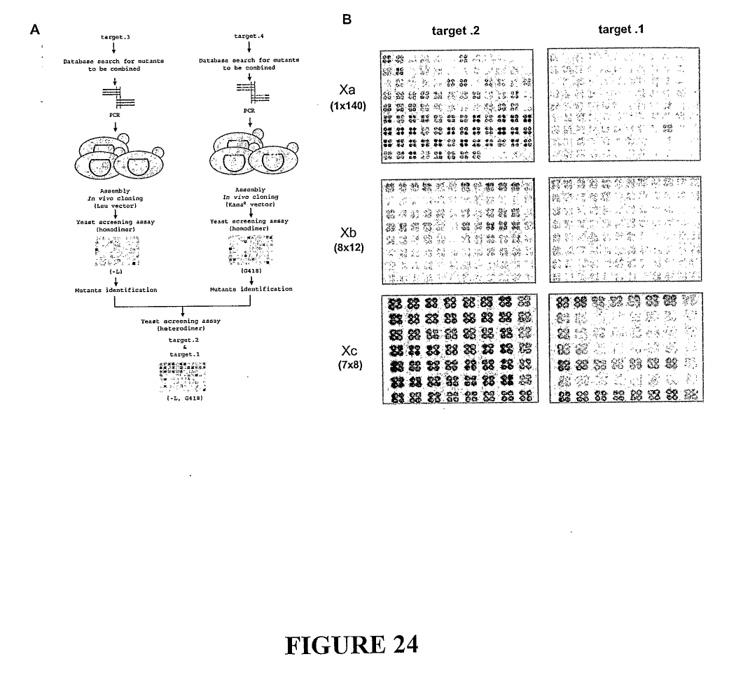

FIG. 24 illustrates the strategy for the making and screening of custom-designed Homing Endonucleases. A. General strategy of primary screen. Appropriate I-CreI derivatives with locally altered specificities are identified in the database. Then, a combinatorial approach is used to assemble these mutants by in vivo cloning. Active combinatorial mutants are identified as homodimers using a yeast screening assay, on either Xx.3 either Xx.4 targets. Heterodimers were screened by co-expression against both non palindromic targets: Xx.2 and Xx.1. B. Heterodimer Screening Examples. Each new endonuclease is screened on both non palindromic targets: Xx.2 and Xx.1, differing at positions ±2 and ±1. The screening is performed as described previously (International PCT Application WO 2004/067736; Arnould et al., J. Mol. Biol., 2006, 355, 443-458); blue staining indicates cleavage.

FIG. 25 illustrates the strategy of activity improvement of custom-designed Homing Endonucleases. A pool of 4 monomers active against Xx.4 is mutagenized by error-prone PCR while its counterpart (monomers active against Xx.3) is used to generate yeast expression strain containing the target Xx.1. The mutagenized library is transformed in a second yeast strain and cloned by in vivo cloning. Screening of heterodimer activity is performed by mating of the two yeast strains. The same procedure is then repeated on the I-CreI variants active against the Xx.3 target.

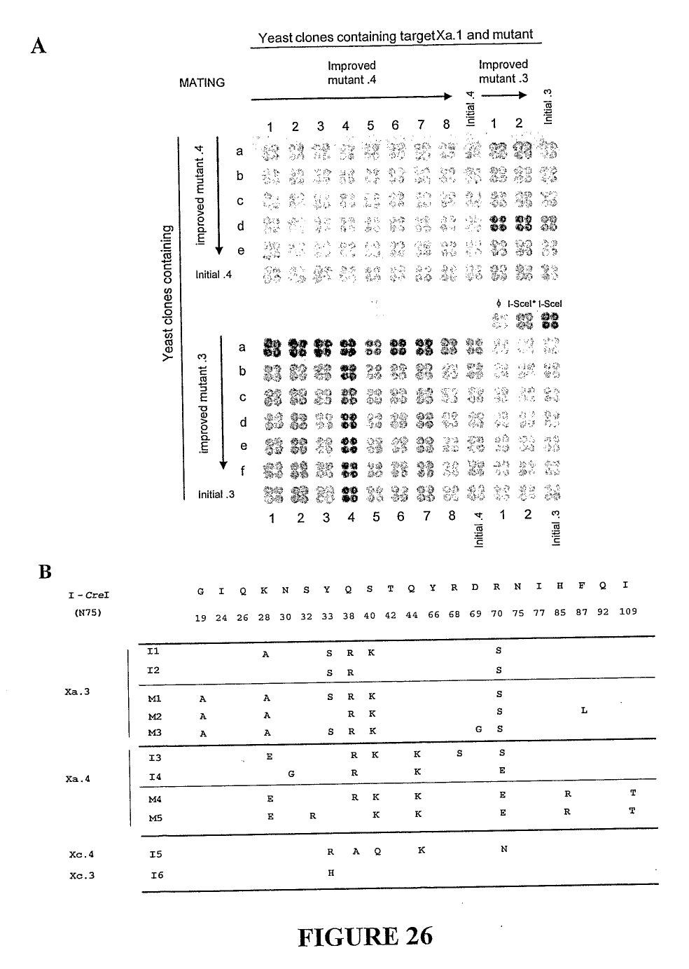

FIG. 26 illustrates the screening and characterisation of improved heterodimers cleaving Xb.1 target. A. Final screen of heterodimers from initials to improve versions. Two forms of I-SceI homing endonuclease are used as a control: I-SceI*, I-SceI variant with poor activity; I-SceI, original I-SceI ORF with strong activity. Ø: yeast strain transformed with empty vector. Initial: representative mutant activity before improvement. Yeast strains containing the improved mutant active either against Xa.3, or against Xa.4 were mated with yeast strain containing the improved mutants and the Xa.1 target. B. Protein sequences of the most active I-CreI variants. I: Protein sequence before activity improvement. M: Protein sequence after random mutagenesis by error-prone PCR.

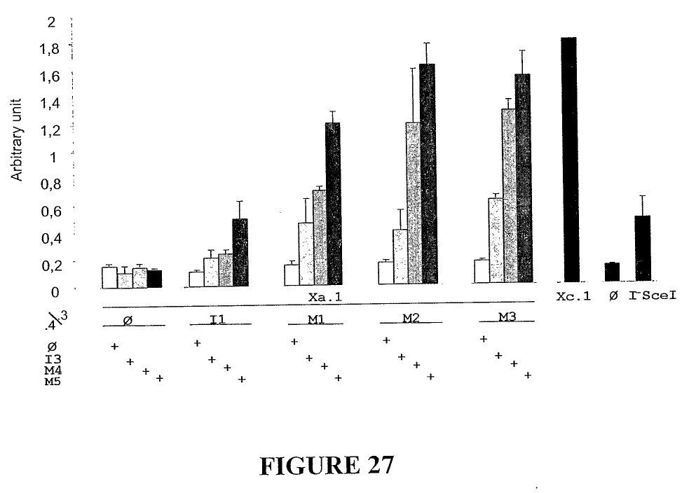

FIG. 27 illustrates the heterodimer activity in mammalian cells. Heterodimer activity was quantified by co-expression assay as described in materials and methods section. Ø: CHO transfected with empty vector. I-SceI: CHO transfected with I-SceI expressing vector. I: mutants before activity improvement. M: mutants after activity improvement. All the target vectors carry an extra sub-optimal 18 bp I-SceI site with poor cleavage efficiency. It is used as control experiment. The I-SceI activity represents the mean obtained with all target vectors.

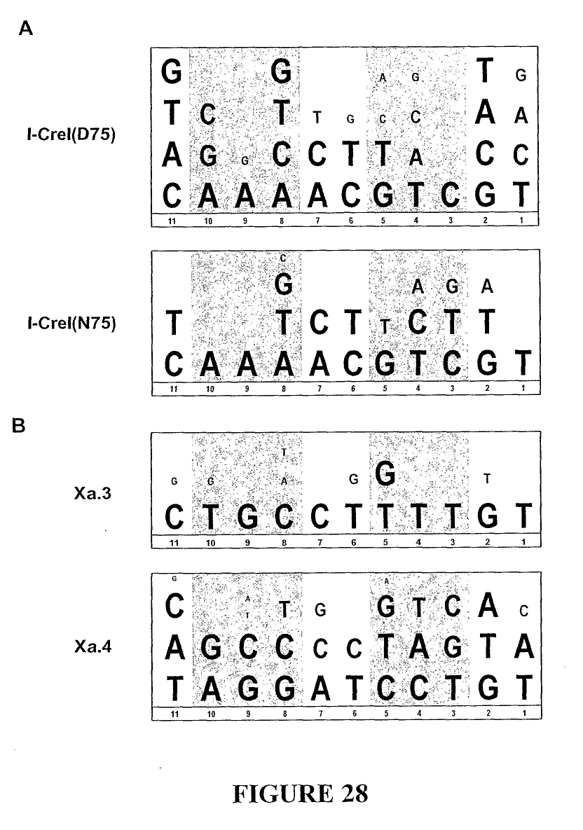

FIG. 28 represents the profiling of combinatorial homodimeric mutants. A. The half site of I-CreI C1221 palindromic target is indicated on the bottom line of each box. The individual nucleotide changes tolerated at each position are indicated. The size of the letters is proportional to the activity of the enyme I-CreI D75N and wild-type. B. The half site of Xa.3 and Xa.4 palindromic targets are indicated on the bottom line. The individual nucleotide changes tolerated at each position are indicated for a mutant.3 and a mutant.4, respectively. The size of the letter is proportional to the activity of the mutants.

### EXAMPLE 1

### Engineering of I-CreI Variants with Modified Specificity in Positions ±8 to ±10

The method for producing meganuclease variants and the assays based on cleavage-induced recombination in mammal or yeast cells, which are used for screening variants with altered specificity, are described in the International PCT Application WO 2004/067736, Arnould et al., J. Mol. Biol., 2006, 355, 443-458, Epinat et al., N.A.R., 2003, 31, 2952-2962 and Chames et al., Nucleic Acids Res., 2005, 33, e178). These assays result in a functional LacZ reporter gene which can be monitored by standard methods.

### A) Material and Methods

### a) Construction of the Ulib4, Ulib5 and Lib4 Libraries

I-CreI wt and I-CreI D75N open reading frames were synthesized, as described previously (Epinat et al., N.A.R., 2003, 31, 2952-2962). Mutation D75N was introduced by replacing codon 75 with aac. Three combinatorial libraries (Ulib4, Ulib5 and Lib4) were derived from the I-CreI D75N protein by replacing three different combinations of residues, potentially involved in the interactions with the bases in positions ±8 to 10 of one DNA target half-site. The diversity of the meganuclease libraries was generated by PCR using degenerated primers harboring a unique degenerated codon (coding for 10 or 12 different amino acids), at each of the selected positions.

The three codons at positions N30, Y33 and Q38 (Ulib4 library) or K28, N30 and Q38 (Ulib5 library) were replaced by a degenerated codon VVK (18 codons) coding for 12 different amino acids: A,D,E,G,H,K,N,P,Q,R,S,T). In consequence, the maximal (theoretical) diversity of these protein libraries was 12³ or 1728. However, in terms of nucleic acids, the diversity was 18³ or 5832. Fragments carrying combinations of the desired mutations were obtained by PCR, using a pair of degenerated primers (Ulib456for and Ulib4rev; Ulib456for and Ulib5rev, FIG. 5B) and as DNA template, the D75N open reading frame (ORF), (FIG. 5A). The corresponding PCR products were cloned back into the I-CreI N75 ORF in the yeast replicative expression vector pCLS0542 (Epinat et al., precited), carrying a LEU2 auxotrophic marker gene. In this 2 micron-based replicative vector, I-CreI variants are under the control of a galactose inducible promoter.

In Lib4, ordered from BIOMETHODES, an arginine in position 70 was first replaced with a serine (R70S). Then positions 28, 33, 38 and 40 were randomized. The regular amino acids (K28, Y33, Q38 and S40) were replaced with one out of 10 amino acids (A,D,E,K,N,Q,R,S,T,Y). The resulting library has a theoretical complexity of 10000 in terms of proteins.

### b) Construction of Target Clones

The C1221 twenty-four bp palindrome (tcaaaacgtcgtacgacgttttga, (SEQ ID NO: 25) is a repeat of the half-site of the nearly palindromic natural I-CreI target (tcaaaacgtcgtgagacagtttgg, SEQ ID NO: 38). C1221 is cleaved as efficiently as the I-CreI natural target in vitro and ex vivo in both yeast and mammalian cells. The 64 palindromic targets were derived from C1221 as follows: 64 pairs of oligonucleotides ((ggcatacaagtttcnnnacgtcgtacgacgtnnngacaatcgtctgtca (SEQ ID NO: 39) and reverse complementary sequences) were ordered form Sigma, annealed and cloned into pGEM-T Easy (PROMEGA) in the same orientation. Next, a 400 bp PvuII fragment was excised and cloned into the yeast vector pFL39-ADH-LACURAZ, also called pCLS0042, and the mammalian vector pcDNA3 derivative (pcDNA3.1-LAACZ), both described previously (Epinat et al., 2003, precited), resulting in 64 yeast reporter vectors (target plasmids).

Alternatively, double-stranded target DNA, generated by PCR amplification of the single stranded oligonucleotides, was cloned using the Gateway protocol (INVITROGEN) into yeast and mammalian reporter vectors.

### c) Yeast Strains

The library of meganuclease expression variants was transformed into the leu2 mutant haploid yeast strain FYC2-6A: alpha, trp1Δ63, leu2Δ1, his3Δ200. A classical chemical/heat choc protocol that routinely gives us 10⁶ independent transformants per μg of DNA derived from (Gietz and Woods, Methods Enzymol., 2002, 350, 87-96), was used for transformation. Individual transformant (Leu⁺) clones were individually picked in 96 wells microplates.13824 colonies were picked using a colony picker (QpixII, GENETIX), and grown in 144 microtiter plates.

The 64 target plasmids were transformed using the same protocol, into the haploid yeast strain FYBL2-7B: a, ura3Δ851, trp1Δ63, leu2Δ1, lys2Δ202, resulting in 64 tester strains.

### d) Mating of Meganuclease Expressing Clones and Screening in Yeast

Meganuclease expressing clones were mated with each of the 64 target strains, and diploids were tested for beta-galactosidase activity, by using the screening assay illustrated on FIG. 2 of Arnould et al., 2006, precited. I-CreI variant clones as well as yeast reporter strains were stocked in glycerol (20%) and replicated in novel microplates. Mating was performed using a colony gridder (QpixII, GENETIX). Mutants were gridded on nylon filters covering YPD plates, using a high gridding density (about 20 spots/cm²). A second gridding process was performed on the same filters to spot a second layer consisting of 64 different reporter-harboring yeast strains for each variant. Membranes were placed on solid agar YPD rich medium, and incubated at 30° C. for one night, to allow mating. Next, filters were transferred to synthetic medium, lacking leucine and tryptophan, with galactose (2%) as a carbon source (and with G418 for coexpression experiments), and incubated for five days at 37° C., to select for diploids carrying the expression and target vectors. After 5 days, filters were placed on solid agarose medium with 0.02% X-Gal in 0.5 M sodium phosphate buffer, pH 7.0, 0.1% SDS, 6% dimethyl formamide (DMF), 7 mM β-mercaptoethanol, 1% agarose, and incubated at 37° C., to monitor β-galactosidase activity. After two days of incubation, positive clones were identified by scanning. The β-galactosidase activity of the clones was quantified using appropriate software. The clones showing an activity against at least one target were isolated (first screening). The spotting density was then reduced to 4 spots/cm² and each positive clone was tested against the 64 reporter strains in quadruplicate, thereby creating complete profiles (secondary screening).

### e) Sequence

The open reading frame (ORF) of positive clones identified during the first and/or secondary screening in yeast was amplified by PCR on yeast colonies using primers: PCR-Gal10-F (gcaactttagtgctgacacatacagg, SEQ ID NO:40) and PCR-Gal10-R (acaaccttgattgcagacttgacc, SEQ ID NO:41) or 5′ggggacaagtttgtacaaaaaaggcaggcttcgaaggagatagaaccatggccaataccaaatataacaaagagttcc3′ (SEQ ID NO: 225) and 5′ggggaccactttgtacaagaaagctgggtttagtcggccgccggggaggatttcttcttctcgc 3′ (SEQ ID NO: 226), from PROLIGO. Briefly, yeast colony is picked and resuspended in 100 μl of LGlu liquid medium and cultures overnight. After centrifugation, yeast pellet is resuspended in 10 μl of sterile water and used to perform PCR reaction in a final volume of 50 μl containing 1.5 μl of each specific primers (100 pmol/μl). The PCR conditions were one cycle of denaturation for 10 minutes at 94° C., 35 cycles of denaturation for 30 s at 94° C., annealing for 1 min at 55° C., extension for 1.5 min at 72° C., and a final extension for 5 min. The resulting PCR products were then sequenced.

### f) Re-Cloning of Primary Hits

The open reading frames (ORFs) of positive clones identified during the primary screening were recloned using the Gateway protocol (Invitrogen). ORFs were amplified by PCR on yeast colonies, as described in e). PCR products were then cloned in: (i) yeast gateway expression vector harboring a galactose inducible promoter, LEU2 or KanR as selectable marker and a 2 micron origin of replication, (ii) a pET 24d(+) vector from NOVAGEN, and (iii) a CHO gateway expression vector pcDNA6.2 from INVITROGEN. Resulting clones were verified by sequencing (MILLEGEN).

### B) Results

I-CreI is a dimeric homing endonuclease that cleaves a 22 bp pseudo-palindromic target. Analysis of I-CreI structure bound to its natural target has shown that in each monomer, eight residues establish direct interactions with seven bases (Jurica et al., 1998, precited). According to these structural data, the bases of the nucleotides in positions +8 to 10 establish specific contacts with I-CreI amino-acids N30, Y33 and Q38 (FIG. 3). Thus, novel proteins with mutations in positions 30, 33 and 38 could display novel cleavage profiles with the 64 targets resulting from substitutions in positions ±8, ±9 and ±10 of a palindromic target cleaved by I-CreI. In addition, mutations might alter the number and positions of the residues involved in direct contact with the DNA bases. More specifically, positions other than 30, 33, 38, but located in the close vicinity on the folded protein, could be involved in the interaction with the same base pairs.

An exhaustive protein library vs. target library approach was undertaken to engineer locally this part of the DNA binding interface. First, the I-CreI scaffold was mutated from D75 to N. The D75N mutation did not affect the protein structure, but decreased the toxicity of I-CreI in overexpression experiments.

Next the Ulib4 library was constructed: residues 30, 33 and 38, were randomized, and the regular amino acids (N30, Y33, and Q38) replaced with one out of 12 amino acids (A,D,E,G,H,K,N,P,Q,R,S,T). The resulting library has a complexity of 1728 in terms of protein (5832 in terms of nucleic acids).

Then, two other libraries were constructed: Ulib5 and Lib4. In Ulib5, residues 28, 30 and 38 were randomized, and the regular amino acids (K28, N30, and Q38) replaced with one out of 12 amino acids (ADEGHKNPQRST). The resulting library has a complexity of 1728 in terms of protein (5832 in terms of nucleic acids). In Lib4, an Arginine in position 70 was first replaced with a Serine. Then, positions 28, 33, 38 and 40 were randomized, and the regular amino acids (8, Y33, Q38 and S40) replaced with one out of 10 amino acids (A,D,E,K,N,Q,R,S,T,Y). The resulting library has a complexity of 10000 in terms of proteins.

In a primary screening experiment, 20000 clones from Ulib4, 10000 clones from Ulib5 and 20000 clones from Lib4 were mated with each one of the 64 tester strains, and diploids were tested for beta-galactosidase activity. All clones displaying cleavage activity with at least one out of the 64 targets were tested in a second round of screening against the 64 targets, in quadriplate, and each cleavage profile was established. Then, meganuclease ORF were amplified from each strain by PCR, and sequenced, and 141 different meganuclease variants were identified.

The 141 validated clones showed very diverse patterns. Some of these new profiles shared some similarity with the wild type scaffold whereas many others were totally different. Results are summarized in FIG. 6. Homing endonucleases can usually accommodate some degeneracy in their target sequences, and the I-CreI N75 scaffold protein itself cleaves a series of 4 targets, corresponding to the aaa, aac, aag, an aat triplets in positions ±10 to ±8. A strong cleavage activity is observed with aaa, aag and aat, whereas AAC is only faintly cut (and sometimes not observed). Similar pattern is found with other proteins, such as I-CreI K28 N30 D33 Q38 S40 R70N75, I-CreI K28 N30 Y33 Q38 S40 R70N75. With several proteins, such as I-CreI R8N30 N33 Q38 D40 S70 N75 and I-CreI K28 N30 N33 Q38 S40 R70N75, aac is not cut anymore.

However, a lot of proteins display very different patterns. With a few variants, cleavage of a unique sequence is observed. For example, protein I-CreI K28 R30G33 T38 S40 R70N75 is active on the “ggg” target, which was not cleaved by wild type protein, while I-CreI Q28 N30 Y33 Q38 R40 S70 N75 cleaves AAT, one of the targets cleaved by I-CreI N75. Other proteins cleave efficiently a series of different targets: for example, I-CreI N28 N30 S33 R38K40 S70 N75 cleaves ggg, tgg and tgt, CreI K28 N30H33 Q38 S40 R70 N75 cleaves aag, aat, gac, gag, gat, gga, ggc, ggg, and ggt. The number of cleaved sequences ranges from 1 to 10. Altogether, 37 novel targets were cleaved by the mutants, including 34 targets which are not cleaved by I-CreI and 3 targets which are cleaved by I-CreI (aag, aat and aac, FIG. 6).

### EXAMPLE 2

### Strategy for Engineering Novel Meganucleases Cleaving a Target from the XPC Gene

A first series of I-CreI variants having at least one substitution in positions 44, 68 and/or 70 of I-CreI and being able to cleave mutant I-CreI sites having variation in positions ±3 to 5 was identified previously (Arnould et al., J. Mol. Biol., 2006, 355, 443-458). The cleavage pattern of the variants is presented in FIG. 7.

A second series of I-CreI variants having at least one substitution in positions 28, 30, 33 or 28, 33, 38 and 40 of I-CreI and being able to cleave mutant I-CreI sites having variation in positions ±8 to 10 was identified as described in example 1. The cleavage pattern of the variants is presented in FIG. 6.

Positions 28, 30, 33, 38 and 40 on one hand, and 44, 68 and 70, on another hand are on a same DNA-binding fold, and there is no structural evidence that they should behave independently. However, the two sets of mutations are clearly on two spatially distinct regions of this fold (FIG. 8) located around different regions of the DNA target. These data suggest that I-CreI comprises two independent functional subunits which could be combined to cleave novel chimeric targets. The chimeric target comprises the nucleotides in positions ±3 to 5 and ±8 to 10 which are bound by each subdomain.

This hypothesis was verified by using targets situated in a gene of interest, the XPC gene. The targets cleaved by the I-CreI variants are 24 bp derivatives of C1221, a palindromic sequence cleaved by I-CreI. However, the structure of I-CreI bound to its DNA target suggests that the two external base pairs of these targets (positions −12 and 12) have no impact on binding and cleavage (Chevalier et al., Nat. Struct. Biol., 2001, 8, 312-316; Chevalier, B. S, and B. L. Stoddard, Nucleic Acids Res., 2001, 29, 3757-3774; Chevalier et al., 2003, J. Mol. Biol., 329, 253-269) and in this study, only positions −11 to 11 were considered. Consequently, the series of targets identified in the XPC gene were defined as 22 bp sequences instead of 24 bp.

Xa.1, Xb.1 and Xc. 1 are 22 bp (non-palindromic) targets located at position 9119, 13521 and 20438, respectively of the human XPC gene (FIGS. 1A, 9 and 23). The meganucleases cleaving Xa.1, Xb.1 or Xc. 1 could be used to correct mutations in the vicinity of the cleavage site (FIG. 1B). Since the efficiency of gene correction decreases when the distance to the DSB increases (Elliott et al., Mol Cell Biol, 1998, 18, 93-101), this strategy would be most efficient with mutations located within 500 bp of the cleavage site. For example, meganucleases cleaving Xc. 1 could be used to correct mutations in Exon 9 (deletion DEL1132AA or insertion insVAL580, FIG. 1A). Alternatively, the same meganucleases could be used to knock-in exonic sequences that would restore a functional XPC gene at the XPC locus (FIG. 1C). This strategy could be used for any mutation downstream of the cleavage site.

Xa.1 is partly a patchwork of the 10TGC_P, 10AGG_P, 5TTT_P and 5CCT_P targets (FIGS. 9 and 23) which are cleaved by previously identified meganucleases (FIGS. 6 and 7). Thus, Xa.1 could be cleaved by combinatorial mutants resulting from these previously identified meganucleases.

Xb.1 is partly a patchwork of the 10GGG_P, 10TGT_P, 5GGG_P and 5TAC_P targets (FIG. 23) which are cleaved by previously identified mega-nucleases (FIGS. 6 and 7). Thus, Xb.1 could be cleaved by combinatorial mutants resulting from these previously identified meganucleases.

Xc.1 is partly a patchwork of the 10GAG_P, 10GTA_P and 5TCT_P targets (FIG. 23) which are cleaved by previously identified meganucleases (FIGS. 6 and 7), and 5GTC_P which is the sequence of C1221 cleaved by I-CreI. Thus, Xc.1 could be cleaved by combinatorial mutants resulting from these previously identified meganucleases.

Therefore, to verify this hypothesis, two palindromic targets, corresponding to the left (Xx.3) and right half (Xx.4) sequences of the identified targets (Xx. 1) were produced (FIGS. 9 and 23). These two derived palindromic targets keep the GTAC sequence from the C1221 palindromic I-CreI target at positions −2 to +2 (FIGS. 9 and 23). Since Xx.3 and Xx.4 are palindromic, they should be cleaved by homodimeric proteins. In a first step, proteins able to cleave the Xx.3 and Xx.4 sequences as homodimers were designed (examples 3 and 4), as illustrated in FIG. 24A. In a second step, the proteins obtained in examples 3 and 4 were co-expressed to obtain heterodimers cleaving Xx.2 and Xx.1, for some heterodimers (example 5), as illustrated in FIG. 24A.

### EXAMPLE 3

### Making of Meganucleases Cleaving Xx.3

This example shows that I-CreI mutants can cut the Xx.3 DNA target sequences derived from the left part of the Xx.2 targets in a palindromic form (FIGS. 9 and 23). Target sequences described in this example are 22 bp palindromic sequences. Therefore, they will be described only by the first 11 nucleotides, followed by the suffix_P; for example, target Xa.3 will be noted also ctgccttttgt_P.

Xa.3 is similar to 5TTT_P in positions ±1, ±2, ±3, ±4, ±5 and ±111 and to 10TGC_P in positions ±1, ±2, ±8, ±9, ±10 and ±11.

Xb.3 is similar to 5GGG_P in positions ±1, ±2, ±3, ±4, ±5 and ±11 and to 10GGG_P in positions ±1, ±2, ±8, ±9, ±10 and ±11

Xc.3 is similar to C1221 in positions ±1, ±2, ±3, ±4, ±5, ±7 and ±11 and to 10GAG_P in positions ±1, ±2, ±7, ±8, ±9, ±10 and ±11

The wild-type I-CreI is known to be tolerant for nucleotide substitutions at positions ±1, ±7 and ±6 (Chevalier et al., J. Mol. Biol., 2003, 329, 253-269; Jurica et al., Mol. Cell., 1998, 2, 469-476). Thus, it was hypothesized that positions +6 and +7 would have little effect on the binding and cleavage activity.

Mutants able to cleave the 5TTT_P and 5GGG_P targets were previously obtained by mutagenesis on I-CreI N75 at positions 44, 68 and 70 or I-CreI S70 at positions 44, 68, 75 and 77, as described in Arnould et al., J. Mol. Biol., 2006, 355, 443-458 (FIG. 7). Mutants able to cleave the 10TGC_P, 10GGG_P, and 10TGA_P targets were obtained by mutagenesis on I-CreI N75 at positions 28, 30, 38, or 30, 33, 38, on I-CreI S70 N75 at positions 28, 33, 38 and 40 and 70, or on I-CreI D75 or N75 at two positions chosen from 28, 30, 32, 33, 38 and 40 (example 1 and FIG. 6). Thus, combining such pairs of mutants would allow for the cleavage of the Xx.3 target.

Some sets of proteins are both mutated at position 70. However, it was hypothesized that two separable functional subdomains exist in I-CreI. That implies that this position has little impact on the specificity in bases 10 to 8 of the target.

Therefore, to check whether combined mutants could cleave the Xa.3 and Xb.3 targets, mutations at positions 44, 68, 70 and/or 75 from proteins cleaving the 5NNN region of the target (5TTT_P and 5GGG_P targets) were combined with the 28, 30, 32, 33, 38 and/or 40 mutations from proteins cleaving the 10NNN region of the targets (10TGC_P and 10GGG_P targets), as illustrated in FIG. 24A.

Xc.3 which is identical to C1221 in positions ±3 to 5 should be cleaved by previously identified mutants cleaving the 10 GAG_P target (no combination of mutations).

### A) Material and Methods

### a) Construction of Target Vector:

The C1221 derived target was cloned as follows: oligonucleotide corresponding to the target sequence flanked by gateway cloning sequence was ordered from Proligo (as example: 5′ tggcatacaagtttctgccttttgtacaaaaggcagacaatcgtctgtca 3′ (SEQ ID NO: 42, for the Xa.3 target). Double-stranded target DNA, generated by PCR amplification of the single stranded oligonucleotide, was cloned using the Gateway^{R} protocol (INVITROGEN) into yeast reporter vector (pCLS1055, FIG. 10). Yeast reporter vector was transformed into S. cerevisiae strain FYBL2-7B (MAT alpha, ura3Δ851, trp1Δ63, leu2Δ1, lys2Δ202).

### b) Construction of Combinatorial Mutants:

I-CreI mutants cleaving 10TGC_P, 10GGG_P, 5TTT_P or 5GGG_P were identified as described in example 1 and FIG. 6, and Arnould et al., J. Mol. Biol., 2006, 355, 443-458 and FIG. 7, respectively for the 10TGC_P, 10GGG_P and the 5TTT_P, 5GGG_P targets. In order to generate I-CreI derived coding sequence containing mutations from both series, separate overlapping PCR reactions were carried out that amplify the 5′ end (amino acid positions 1-43) or the 3′ end (positions 39-167) of the I-CreI coding sequence. For both the 5′ and 3′ end, PCR amplification is carried out using primers specific to the vector (pCLS0542, FIG. 11): Gall OF or Ga110R and primers specific to the I-CreI coding sequence for amino acids 39-43 (assF 5′-ctannnttgaccttt-3′ (SEQ ID NO: 43) or assR 5′-aaaggtcaannntag-3′ (SEQ ID NO: 44)) where nnn codes for residue 40. The resulting PCR products contain 1 Sbp of homology with each other and approximately 100-200 bp of homology with the 2 micron-based replicative vectors, pCLS0542, marked with the LEU2 gene and pCLS1107, containing a kanamycin resistant gene.

Thus, to generate an intact coding sequence by in vivo homologous recombination in yeast, approximately 25 ng of each of the two overlapping PCR fragments and 25 ng of the pCLS0542 vector DNA linearized by digestion with NcoI and EagI or 25 ng of the pCLS1107 vector DNA linearized by digestion with DraIII and NgoMIV were used to transform the yeast Saccharomyces cerevisiae strain FYC2-6A (MATα, trp1Δ63, leu2Δ1, his3Δ200) using a high efficiency LiAc transformation protocol (Gietz, R. D. and R. A. Woods, Methods Enzymol, 2002, 350, 87-96). Combinatorial mutants can advantageously be generated as libraries: PCR reactions were pooled in equimolar amounts and transformed into yeast together with the linearized plasmid. Transformants were selected on either synthetic medium lacking leucine (pCLS0542) or rich medium containing G418 (PCLS1107). Colonies were picked using a colony picker (QpixII, Genetix), and grown in 96 well microtiter plates.

### c) Mating of Meganuclease Expressing Clones and Screening in Yeast

The experimental procedure is as described in example 1, except that a low gridding density (about 4 spots/cm²) was used.

### c) Sequencing of Mutants

To recover the mutant expressing plasmids, yeast DNA was extracted using standard protocols and used to transform E. coli. Sequencing of mutant ORF was then performed on the plasmids by MILLEGEN SA. Alternatively, the ORFs of positive clones identified during the primary screening in yeast were amplified by PCR on yeast DNA extract from colonies (Akada et al., Biotechniques, 2000, 28(4): 668-70, 672, 674) using primers 5′ ggggacaagtttgtacaaaaaagcaggcttcgaaggagatagaaccatggccaataccaaatataacaaagagttcc 3′ (SEQ ID NO: 225) and 5′ggggaccactttgtacaagaaagctgggtttagtcggccgccgggaggatttcttcttctcgc 3′ (SEQ ID NO: 226) from PROLIGO and sequencing was performed directly on PCR product by MILLEGEN. PCR products were cloned either in (i) yeast gateway expression vectors harboring a galactose inducible promoter, LEU2 or KanR as selectable marker and a 2 micron origin of replication, (ii) CHO gateway expression vector pcDNA6.2 from INVITROGEN. Resulting clones were verified by sequencing (MILLEGEN).

### B) Results

I-CreI N75 mutants cutting the 10TGC_P (ctgcacgtcgt_P) target and I-CreI N75 (Q44, R68, R70) mutants cutting the 5TTT_P (caaaactttgt_P) target were combined, resulting in combinatorial mutants that were screened against the Xa.3 target (ctgccttttgt_P).

I-CreI N75 mutants cutting the 10GGG_P target and I-CreI N75 mutants cutting the 5GGG_P target were combined, resulting in combinatorial mutants that were screened against the Xb.3 target.

At least twice the diversity of each library was screened. No correlation was observed between the number of active mutants identified and the number of combinations tested.

The Xc.3 target sequence contained the wild type sequence GTC at positions ±5, ±4 and ±3; therefore the combinatorial approach was not necessary to generate specific variants towards this target. I-CreI variants previously identified with altered substrate specificity towards bases ±10, ±9 and ±8 were directly screened against the Xc.3 DNA target.

TABLE XVI
Mutants used for combinatorial construction for the palindromic target Xx.3

| | Number of I-CreI | | | | Number | |
|---|---|---|---|---|---|---|
| | mutants used for the | | | | of combined | |
| | combinatorial reaction | | | Tested clones | unique | |
| Targets | 10NNN_P X 5NNN_P | | diversity | (X diversity) | positive clones | |
| Xa.3 | 510 | na* | 1099 | 2 | 8 | (0.7%) |
| | 31 | 19 | | | | |
| Xb.3 | 45 | 32 | 1440 | 2 | 30 | (2%) |
| Xc.3 | 811 | na* | 811 | na* | 21 | (2%) |

na: non applicable
*only I-CreI variants with altered specificity towards nucleotides ±10, ±9 and ±8 were screened.

Eight combinatorial mutants were found cleave the Xa.3 target (Table XVI). Two of the mutants cleaving the Xa.3 target have the following sequence:
I-CreI K28, N30, S33, R38, S40, S70 and N75 (called KNSRSSN), obtained by combination of I-CreI K28, N30, S33, R38, S40 and I-CreI S70,N75, and
I-CreI A28, N30, S33, R38, K40, S70 and N75 (called ANSRKSN), obtained by combination of I-CreI K28, N30, S33, R38, K40 and I-CreI S70, N75.

Thirty combinatorial mutants were found to cleave the Xb.3 target (Table XVI).

Among the mutants cutting the 10GAG_P target which were tested, twenty one were found to cleave the Xc.3 target (Table XVI).

Results were confirmed in a secondary screen (FIG. 12) and the predicted amino acid primary structure was confirmed by sequencing.

### EXAMPLE 4

### Making of Meganucleases Cleaving Xx.4

This example shows that I-CreI variants can cleave the Xx.4 DNA target sequence derived from the right part of the Xx.2 target in a palindromic form (FIGS. 9 and 23). All target sequences described in this example are 22 bp palindromic sequences. Therefore, they will be described only by the first 11 nucleo-tides, followed by the suffix_P; for example, Xa.4 will be called taggatcctgt_P (SEQ ID NO: 37).

Xa.4 is similar to 5CCT_P in positions ±1, ±2, ±3, ±4, ±5 and ±7 and to 10AGG_P in positions ±1, ±2, ±7, ±8, ±9 and ±10. It was hypothesized that positions 16 and +11 would have little effect on the binding and cleavage activity.

Xb.4 is similar to 5TAC_P in positions ±1, ±2, ±3, ±4, ±5, ±6 and ±11 and to 10TGT_P in positions ±1, ±2, ±6, ±8, ±9, ±10, and ±11. It was hypothesized that positions ±7 would have little effect on the binding and cleavage activity.

Xc.4 is similar to 5TCT_P in positions ±1, ±2, ±3, ±4, ±5, ±7 and ±11 and to 10GTA_P in positions ±1, ±2, ±7, ±8, ±9, ±10, and ±11. It was hypothesized that positions +6 would have little effect on the binding and cleavage activity.

Mutants able to cleave the 5CCT_P, 5TAC_P and 5TCT_P targets were previously obtained by mutagenesis on I-CreI N75 at positions 44, 68 and 70 or I-CreI S70 at positions 44, 68, 75 and 77, as described in Arnould et al., J. Mol. Biol., 2006, 355, 443-458 (FIG. 7). Mutants able to cleave the 10AGG_P, 10TGT_P, and 10GTA_P targets were obtained by mutagenesis on I-CreI N75 at positions 28, 30, 38, or 30, 33, 38, on I-CreI S70 N75 at positions 28, 33, 38 and 40 and 70, or on I-CreI D75 or N75 at two positions chosen from 28, 30, 32, 33, 38 and 40 (example 1 and FIG. 6). Thus, combining such pairs of mutants would allow for the cleavage of the Xx.4 target.

Some sets of proteins are both mutated at position 70. However, it was hypothesized that I-CreI comprises two separable functional subdomains. That implies that this position has little impact on the specificity in base 10 to 8 of the target.

Therefore, to check whether combined mutants could cleave the Xx.4 target, mutations at positions 44, 68, 70 and/or 75 from proteins cleaving 5CCT_P, 5TAC_P and 5TCT_P targets were combined with the 28, 30, 32, 33, 38, and/or 40 mutations from proteins cleaving 10AGG_P, 10TGT_P, and 10GTA_P targets, as illustrated in FIG. 24A.

### A) Material and Methods

See example 3.

### B) Results

I-CreI combined mutants were constructed by associating mutations at positions 44, 68 and 70 with the 28, 30, 33, 38 and 40 mutations on the I-CreI N75 scaffold. Combined mutants were screened against the Xx.4 DNA targets At least twice the diversity of each library was screened. No correlation was observed between the number of active mutants identified and the number of combinations tested. Two percent of the hybrid mutants appear to be functional for the Xb.4 DNA targets while as many as 55% were active against Xa.4. After secondary screening and sequencing, 104, 8 and 4 different cleavers were identified, for the Xa.4, Xb.4 and Xc.4 target, respectively (Table XVII).

TABLE XVII
Mutants used for combinatorial construction for the palindromic target Xx.4

| | Number of I-CreI | | | | Number | |
|---|---|---|---|---|---|---|
| | mutants used for the | | | | of combined | |
| | combinatorial reaction | | | Tested clones | unique | |
| Targets | 10NNN_P X 5NNN_P | | diversity | (X diversity) | positive clones | |
| Xa.4 | 9 | 21 | 189 | 4 | 104 | (55%) |
| Xb.4 | 7 | 46 | 322 | 5 | 8 | (2%) |
| Xc.4 | 3 | 47 | 141 | 3 | 24 | (17%) |

na: non applicable
*only I-CreI variants with altered specificity towards nucleotides 10, 9 and 8 were screened.

I-CreI N75 mutants cutting the 10AGG_P (caggacgtcgt_P; SEQ ID NO: 32) target (amino acids at positions 28, 30, 33, 38 and 40 are indicated) and I-CreI N75 mutants cutting the 5CCT_P (caaaaccctgt_P; SEQ ID NO: 34) target (amino acids at positions 44, 68 and 70 are indicated) are listed in Table XVIII. 39 of the 104 positives are presented in FIG. 13 and Table XVIII.

TABLE XVIII
Cleavage of the Xa.4 target by the combined variants*

| | | Amino acids at positions 28, 30, 33, 38 and 40 (ex: ENYRKstands for E28, N30, Y33, R38 and K40) | | | | | | | | | |
|---|---|---|---|---|---|---|---|---|---|---|---|
| | | ENYRK | ENRRK | SNYRK | KGYGS | KGYHS | KGYRS | KGYTS | KDAHS | KDHKS | KDRGS |
| Amino | KGA | | | | | | | | | | |
| acids at | KSN | | | | | | | | | | |
| positions | KQD | | | | | | + | | | | |
| 44, 68 and | KRE | | + | | + | + | + | | | | |
| 70 (ex: | KNH | | | | | | | | | | |
| KGA stands | KSA | | | | | | | | | | |
| for K44, | KRN | | | | | + | | | | | |
| G68 and | KGS | | | | | + | + | | | | |
| A70) | KNN | | | | | + | | | | | |
| | KNG | | | | | | | | | | |
| | KGG | | | | | | + | | | | |
| | KTH | | | | | | + | | | | |
| | RRD | | | | | | | | | | |
| | KRS | | | | + | + | | | | | |
| | KRT | | | | | | | | | | |
| | KSD | | | | | + | + | | | | |
| | KSS | + | | + | | | + | | | | |
| | KHS | | | | | + | + | | | | |
| | KTS | | + | | | + | + | | | | |
| | KRD | | + | + | | + | + | | | | |
| | KAG | | | | | + | + | | | | |
| | KAS | | + | + | | + | | | | | |
| | KAQ | | | | | | + | | | | |
| | KAN | | | | | | + | | | | |
| | KQS | | | | | | + | | | | |
| | KPS | | | | | + | | | | | |
| | KNA | | | | | + | | | | | |
| | KHD | | | | | + | + | | | | |

+functional combination.
*all proteins have also a D75N mutation

### EXAMPLE 5

### Making of Meganucleases Cleaving Xx.2 and Xx.1.1

I-CreI mutants able to cleave each of the palindromic Xx.2 derived targets Xx.3 and Xx.4, were identified in examples 3 and 4. A subset of pairs of such mutants (one cutting Xx.3 and one cutting Xx.4), were co-expressed in yeast. Upon co-expression, there should be three active molecular species, two homodimers, and one heterodimer. It was assayed whether the heterodimers that should be formed could cleave the Xx.1 and Xx.2 targets as depicted in FIG. 24A.

### A) Material and Methods

### a) Cloning of Mutants in Kanamycin Resistant Vector:

In order to co-express two I-CreI mutants in yeast, mutants cutting the Xx.3 sequence were subcloned in a kanamycin resistant yeast expression vector (PCLS1107, FIG. 14).

Mutants were amplified by PCR reaction using primers common for leucine vector (pCLS0542) and kanamycin vector (pCLS1107) (Ga110F and Ga110R). Approximately 25 ng of PCR fragment and 25 ng of vector DNA (pCLS1107) linearized by digestion with DraIII and NgoMIV are used to transform the yeast Saccharomyces cerevisiae strain FYC2-6A (MATα, trp1Δ63, leu2Δ1, his3Δ200) using a high efficiency LiAc transformation protocol. An intact coding sequence for the I-CreI mutant is generated by in vivo homologous recombination in yeast.

### b) Mutants Coexpression:

Yeast strain expressing a mutant cutting the Xx.4 target was transformed with DNA coding for a mutant cutting the Xx.3 target in pCLS1107 expression vector. Transformants were selected on −L Glu+G418 medium.

### c) Mating of Meganucleases Coexpressing Clones and Screening in Yeast:

The experimental procedure is as described in example 1, except that a low gridding density (about 4 spots/cm²) was used.

### B) Results:

The Table XIX summarizes the number of total and active heterodimers tested by co-expression in yeast against the targets Xx.2 and Xx.1.

TABLE XIX
Mutants used for the heterodimeric assay for each XPC target

| | Number of XPC | | | |
|---|---|---|---|---|
| | mutants used for | | Number of positives | |
| | heterodimeric assay I | | heterodimer combinations | |
| Targets | target.3 × target.4 | | target.2 | target.1 |
| Xa | 2 | 104 | 156 | 15 |
| Xb | 12 | 8 | 43 | 0 |
| Xc | 7 | 8 | 56 | 41 |

In all cases, heterodimers with cleavage activity for the target.2 were identified (FIGS. 15a, 15b and 24B). All of the heterodimers tested were active against Xc.2 while 75% and 45% were able to cleave the Xa.2 and Xb.2 targets respectively. On the other hand, while the 4 central nucleotides sequences TTGA, GAAA, and ACAC found respectively in Xa.1, Xb.1 and Xc.1 targets did not impact the I-CreI cleavage activity when inserted in the I-CreI C1221 palindromic target, only a sub-fraction of the active heterodimers against Xa.2 and Xc.2 were able to cleave the Xa.1 and Xc.1 targets, respectively, while no cutters were found for the Xb.1 target (FIGS. 15a, 15b and 24B). The influence of the central sequence has been explained by its particular topology. Structure analysis revealed that this DNA region of the target is a region of maximal DNA bending with a curvature of around 50° resulting in base twisting and unstacking near the scissile phosphate groups (Chevalier et al., J. Mol. Biol., 2003329, 253-269). However, a precise mechanism explaining why certain sequences at the four central positions are compatible with cleavage activity and some others are not has still to be described.

Examples of functional combinations for the Xa.1 target are presented in Tables XX and XXI. As a general rule, functional heterodimers cutting Xx.1 sequence were always obtained when the two expressed proteins gave a strong signal as homodimer. Moreover, while many mutants are still very active against Xc.1, the mutants capable of cleaving the Xa.1 target displayed a weak activity

TABLE XX
Combinations that resulted in cleavage of the Xa.2 target (+) or Xa.2 and Xa.1(++) targets when expressed with KNSRSS

| | | Amino acids at positions 28, 30, 33, 38 and 40 (ex: ENYRKstands for E28, N30, Y33, R38 and K40) | | | | | | | | | |
|---|---|---|---|---|---|---|---|---|---|---|---|
| | | ENYRK | ENRRK | SNYRK | KGYGS | KGYHS | KGYRS | KGYTS | KDAHS | KDHKS | KDRGS |
| Amino | KGA | | | | | | | | | | |
| acids at | KSN | | | | | | | | | | |
| positions | KQD | | | | | | + | | | | |
| 44, 68 and | KRE | | + | | + | ++ | ++ | | | | |
| 70 (ex: | KNH | | | | | | | | | | |
| KGA stands | KSA | | | | | | | | | | |
| for K44, | KRN | | | | | + | | | | | |
| G68 and | KGS | | | | | + | + | | | | |
| A70) | KNN | | | | | + | | | | | |
| | KNG | | | | | | | | | | |
| | KGG | | | | | | + | | | | |
| | KTH | | | | | | ++ | | | | |
| | RRD | | | | | | | | | | |
| | KRS | | | | + | + | | | | | |
| | KRT | | | | | | | | | | |
| | KSD | | | | | + | + | | | | |
| | KSS | ++ | | ++ | | | ++ | | | | |
| | KHS | | | | | + | | | | | |
| | KTS | | | | | + | ++ | | | | |
| | KRD | | + | ++ | | + | + | | | | |
| | KAG | | | | | ++ | | | | | |
| | KAS | | + | ++ | | + | | | | | |
| | KAQ | | | | | | + | | | | |
| | KAN | | | | | | + | | | | |
| | KQS | | | | | | + | | | | |
| | KPS | | | | | + | | | | | |
| | KNA | | | | | + | | | | | |
| | KHD | | | | | + | + | | | | |

TABLE XXI
Combinations that resulted in cleavage of the Xa.2 target (+) or Xa.2 and Xa.1 (++) targets when expressed with ANSRKS

| | | Amino acids at positions 28, 30, 33, 38 and 40 (ex: ENYRKstands for E28, N30, Y33, R38 and K40) | | | | | | | | | |
|---|---|---|---|---|---|---|---|---|---|---|---|
| | | ENYRK | ENRRK | SNYRK | KGYGS | KGYHS | KGYRS | KGYTS | KDAHS | KDHKS | KDRGS |
| Amino | KGA | | | | | | | | | | |
| acids at | KSN | | | | | | | | | | |
| positions | KQD | | | | | | + | | | | |
| 44, 68 and | KRE | | + | | + | ++ | ++ | | | | |
| 70 (ex: | KNH | | | | | | | | | | |
| KGA stands | KSA | | | | | | | | | | |
| for K44, | KRN | | | | | | | | | | |
| G68 and | KGS | | | | | + | + | | | | |
| A70) | KNN | | | | | + | | | | | |
| | KNG | | | | | | | | | | |
| | KGG | | | | | | | | | | |
| | KTH | | | | | | ++ | | | | |
| | RRD | | | | | | | | | | |
| | KRS | | | | | + | | | | | |
| | KRT | | | | | | | | | | |
| | KSD | | | | | + | + | | | | |
| | KSS | ++ | | ++ | | | ++ | | | | |
| | KHS | | | | | + | + | | | | |
| | KTS | | + | | | + | ++ | | | | |
| | KRD | | + | ++ | | + | + | | | | |
| | KAG | | | | | ++ | + | | | | |
| | KAS | | + | ++ | | + | | | | | |
| | KAQ | | | | | | + | | | | |
| | KAN | | | | | | + | | | | |
| | KQS | | | | | | + | | | | |
| | KPS | | | | | + | | | | | |
| | KNA | | | | | + | | | | | |
| | KHD | | | | | + | + | | | | |

### EXAMPLE 6

### Optimization of the Cleavage Activity of the Meganucleases

### A) Material and Methods

### Activity Improvement

Error-prone PCR was used to introduce random mutations in a pool of 4 chosen mutants. Libraries were generated by PCR using either Mn²⁺, or by two-steps process using dNTPs derivatives 8-oxo-dGTP and dPTP as described in the protocol from JENA BIOSCIENCE GmbH for the JBS dNTP-Mutagenesis kit. Primers used are: preATGCreFor (5′-gcataaattactatacttctatagacacgcaaacacaaatacacagcggccttgccacc-3′, SEQ ID NO: 227) and ICreIpostRev (5′-ggctcgaggagctcgtctagaggatcgctcgagttatcagtcggccgc-3′, SEQ ID NO: 228). For the first round of activity improvement, the new libraries were cloned in vivo in yeast in the linearized kanamycin vector (pCLS1107, FIG. 14) harboring a galactose inducible promoter, a KanR as selectable marker and a 2 micron origin of replication. For the second round of activity improvement, a 2 micron-based replicative vector marked with the LEU2 gene is used. Positives resulting clones were verified by sequencing (MILLEGEN).

### B) Results

Since, the decrease in the cleavage activity between the target.2 and the target .1 (FIG. 24B) could be a consequence of a bias towards the 4 central nucleotides gtac introduced during the process of making meganucleases, it was hypothesized that a weak, activity could be subsequently improved by compensatory mutations. The FIG. 25 depicts the general workflow of the strategy, which was used to refine the proteins activity. The process of optimization of the cleavage activity is performed in two steps. First, after identification of the functional heterodimers for the target sequence Xa.1, a pool of 2 to 4 monomers initially identified by screening against Xa.4 target (mutant .4) is mutagenized by error-prone PCR, while its counterpart (e.g. the mutants identified by screening against the Xa.3 target) remain unmodified. The mutagenized library is then transformed in yeast and the screening is performed by mating with a yeast strain containing the target Xa.1 and the non-mutagenized mutant (active on Xa.3 in this case). About 2300 clones are usually tested, since this number of clones was sufficient to find mutants with improved activity. Once the combinations giving enhanced activity are identified, the same procedure is repeated to optimize the mutants interacting with the Xa.3 target (mutant .3). After error-prone PCR, the library is screened against Xa.1 target by mating with yeast containing the target and the improved mutants previously identified. At the end of this procedure, protein heterodimers are obtained, wherein both monomers have been optimized for a defined protein combination versus one target sequence. Finally, the improved variants are validated in a crossed experiment as shown in FIG. 26A (example of the cleavage activity in yeast of a subset of heterodimers against the target Xa.1 obtained after monomers improvements). Five improved mutants cleaving Xa.4 and 6 improved mutants cleaving Xa.3 were tested in combination with 2 and 8 improved mutants cleaving Xa.3 and Xa.4 respectively, against the Xa.1 target. The original mutants used for the error-prone PCR were incorporated in the experiment as controls. No activity could be detected against the target Xa.1 with homodimers alone as shown by the white colonies obtained after mating of yeast clones containing mutants cleaving only Xa.3 or Xa.4. In contrast, a strong cleavage activity can be visualized when mating occurred between yeast clones containing both mutant species indicating that the cleavage activity resulted from heterodimer formation. Furthermore, the strongest activity is achieved when both monomers have been optimized as compared to the activity achieved with heterodimers for which only one monomer activity has been improved.

The 6 combinations giving the strongest cleavage activity were selected and the ORFs were sequenced. Interestingly, the 6 most active heterodimers resulted from 6 different combinations of 3 independent mutants cleaving Xa.3 with 2 different mutants cleaving Xa.4. The FIG. 26B shows the sequence of the original proteins used for the improvement process (I1-I4)) and their respective optimized proteins (M1-M5). The 15 and 16 sequences in FIG. 26B are the protein sequences of the 2 monomers giving the best activity on Xc.1 target by co-expression assay. As the cleavage of this target was highly efficient, no further activity refinement was needed.

The protein sequence analysis of the best cutters does not reveal any particular protein domains affected by the mutation process. As compared with the original sequence, the error-prone PCR introduced mutations at positions 19, 69 and 87 in the ORF of active mutants cleaving Xa.3 target and mutations at positions 32, 85 and 109 in the coding sequence of the mutants cleaving Xa.4 target. The positions 33 and 38 were also reverted in the protein sequence of the M2 and M5 proteins. Interestingly, the amino acid at position 19 was mutated in all proteins with activity towards the Xa.3 target. This position is part of the catalytic site (Chevalier et al., Biochemistry, 2004, 43, 14015-14026) and with the adjacent Asp20, is involved in the metal cation binding. This is the only mutation which can be directly linked to improvement of the catalytic mechanism. The mutations in positions 32 and 69 affect the protein-DNA interface and the mutations in positions 85 and 87 affect the hydrophobic core. The other mutations affect mainly the core protein indicating that a mechanism of propagated conformational change is responsible for the improved activity. This long range effect could, for example, improve the binding affinity of the mutant and therefore increase its cleavage activity.

These results demonstrate that the combinatorial approach associated to random mutagenesis allows the rapid and efficient production of custom-designed endonucleases for specific DNA substrates.

### EXAMPLE 7

### The Heterodimeric Meganucleases are Functional in Mammalian Cells

### A) Material and Methods

### Mammalian Cells Assays

CHO cells were transfected with Polyfect® transfection reagent according to the supplier (QIAGEN) protocol. 72 hours after transfection, culture medium was removed and 150 μl of lysis/revelation buffer added for β-galactosidase liquid assay (1 liter of buffer contains 100 ml of lysis buffer (Tris-HCl 10 mM pH7.5, NaCl 150 mM, Triton X100 0.1%, BSA 0.1 mg/ml, protease inhibitors), 10 ml of Mg 100× buffer (MgCl₂ 100 mM, β-mercaptoethanol 35%), 110 ml ONPG (8 mg/ml) and 780 ml of sodium phosphate 0.1 M pH7.5). After incubation at 37° C., OD was measured at 42 0 nm. The entire process is performed on an automated Velocity11 BioCel platform.

### B) Results

The hybrid meganucleases active in yeast were tested in mammalian cells by transient co-transfection of CHO cells with a target vector and meganuclease expression vectors. For this purpose, subsets of mutants and their corresponding targets (Xa.1 and Xc.1) were cloned into appropriate vectors as described in example 1, and the meganuclease-induced recombination efficiency was measured by a standard, quantitative ONPG assay that monitors the restoration of a functional β-galactosidase gene, as described previously (International PCT Application WO 2006/097853; Arnould et al., J. Mol. Biol., 2006, 355, 443-458; Smith et al., Nucleic Acids Res., Epub 27 November 2006). FIG. 27 shows the cleavage activity in CHO-K1 cells for the most active hybrid proteins identified in yeast. All target plasmids carry a minimal I-SceI site of 18 bp as an internal control; therefore all mutants can be compared to the I-SceI activity in the same experimental conditions. This 18 bp I-SceI site is weakly cleaved by the I-SceI protein and was chosen in order to avoid saturation of the signal in CHO cells. No cleavage activity could be detected when a single protein is expressed as judged by their background level activity (FIG. 27; Ø/Ø, Ø/I1 to Ø/M3, and Ø/I3 to Ø/M5) indicating that activity is dependent on heterodimer formation. Furthermore, the co-expression of the initial combinatorial mutants (I1, I3) has a very weak activity which is virtually not distinguishable from the background level. The cleavage activity increases as soon as one of the protein partners has been improved. Finally, the most efficient cleavage activities for the Xa.1 target is achieved by co-expression of 2 improved proteins and reaches an activity level similar to the activity of the mutants identified for the Xc. 1 target. These results confirm the data obtained in yeast in which co-expression of the initial combinatorial mutants lead to efficient cleavage of the Xc.1 target without the need of improvement activity while the mutants directed towards Xa.1 had poor activity. However a high activity for the Xa.1 target could be generated after introduction of compensatory mutations. When monomers are expressed on their own, neither toxicity nor cleavage, as shown in FIG. 27, could be detected, showing that the mutants designed towards Xa and Xc keep the original specificity of the I-CreI homing endonuclease or at least a specificity compatible with cell survival.

### EXAMPLE 8

### Analysis of the Individually Mutated Target Cleavage Pattern

The degeneracy at individual positions of the I-CreI target has been previously assayed using in vitro site selection in which variant DNA targets cleavable by wtI-CreI could be recovered (Argast et al., J. Mol. Biol., 1998, 280, 345-353). It indicates that most nucleotide positions in the site can be mutated without loss of binding or cleavage. However no exhaustive study was done. In order to compare the improved mutants towards Xa.1 target with I-CreI homing endonuclease, we have generated all possible targets carrying individual mutation were generated for Xa.3, Xa.4 and the palindromic I-CreI target C1221. The protein scaffold used to generate all the mutants carries a D to N mutation at position 75. This mutation was introduced in order to decrease the energetic strains caused by the replacement of the basic residues at positions 68 and 70 in the libraries. It was shown previously that the D75N mutation decreased the toxicity of over-expressed I-CreI protein without affecting the protein basic folding properties and activity (Arnould et al., J. Mol. Biol., 2006, 355, 443-458). The extent of degeneracy of base-pair recognition of wild type I-CreI (wt I-CreI), the initial protein scaffold I-CreI(N75) and the combinatorial mutants was assayed by measuring their cleavage efficiency in yeast on their respective palindromic targets (C1221, Xa.3 and Xa.4) carrying individual site mutations. I-CreI D75 (wt) and I-CreI N75

As shown in FIG. 28A, the wild type protein (I-CreI-D75) can accommodate many individual mutations on its palindromic target (the font size of each nucleotide is proportional to the cleavage efficiency). The positions ±11, ±8, ±2, and ±1 are the most tolerant positions since none of the 4 nucleotides at these positions affect the cleavage efficiency. In contrast, positions ±9 and ±3 accept very few changes. In comparison, the I-CreI scaffold used to generate the mutants (I-CreI-N75) reveals a different pattern and seems to be less tolerant to point mutations in its target. The most stringent differences are seen at positions ±1, ±3, ±10, and ±1. The only bases allowing target cleavage are ±1T, ±9A and ±10A while G and A at position ±11 inhibit the cleavage of the target by I-CreI(N75) protein. For both wt I-CreI and I-CreI(N75), C or A and T or C at positions ±7 and ±6 respectively, allow maximum cleavage efficiency. Altogether wt I-CreI and I-CreI(N75) cleave respectively 26 and 14 targets carrying single mutation. Based on this data, and if it is assumed that each half target can have an independent impact on the global cleavage efficiency, wt I-CreI should be able to cleave a maximum of 676 (26×26) out of 1156 (34×34) non palindromic targets with individual mutations while I-CreN75 should cut only 196 (14×14). Probably, the restricted pattern of the I-Cre(N75) observed in this study could explain the absence of toxicity of this I-CreI variant. As suggested in early studies (Chevalier et al., J. Mol. Biol., 2003, 329, 253-269; Seligman et al., Genetics, 1997, 147, 1353-1664), the tolerance of individual nucleotide polymorphisms allows I-CreI(D75) to recognize a defined population of targets and facilitates the repeated horizontal transmission of the intron during evolution.

### Combinatorial Mutants Cleaving the Xa.3 and Xa.4 Targets

The FIG. 28B shows the results obtained with the best combinatorial mutants cleaving the Xa.3 and Xa.4 targets. The mutant cleaving the Xa.4 target is much more permissive than the mutant cleaving the Xa.3 target for individual mutation on their respective targets. The combinatorial Xa.4 mutant is able to cleave 24 palindromic targets of which 20 are strongly cleaved. The pattern of single-base substitutions tolerated by the Xa.4 mutants is very similar to that of wt I-CreI towards its own target. In contrast, custom-designed variant towards Xa.3 targets revealed to have the highest stringency regarding individual mutations as only 3 palindromic targets are highly cleavable. This mutant is capable of cleaving only 8 (of which 4 are efficiently cleaved) out of the 34 palindromic targets. Except at the position ±5 which can tolerate G and T, efficient cleavage is achieved only for the target on which it has been selected. As none of the mutants tested displayed degeneracy greater than the wt I-CreI natural protein, these data provide evidence that the combinatorial approach used to generate the mutants results in a change of substrate specificity instead of a simple relaxation of the protein specificity towards its target. Furthermore, the combinatorial mutants have been checked against the parental sequences and the original I-CreI target sequence and none of the proteins tested cleaves these targets. Altogether, the heterodimer designed to cleave the Xa.1 target should be able to cut a maximum of 192 (8×24) out of the 1156 non palindromic targets with individual mutations.

1. An I-CreI variant having at least two substitutions, one in each of the two functional subdomains of the LAGLIDADG (SEQ ID NO: 229) core domain situated from positions 26 to 40 and 44 to 77 of I-CreI, wherein said variant can cleave a DNA target sequence from a xeroderma pigmentosum gene, and wherein said variant is obtained by a method comprising:
(a) constructing a first series of I-CreI variants having at least one substitution in a first functional subdomain of the LAGLIDADG (SEQ ID NO: 229) core domain situated from positions 26 to 40 of I-CreI,
(b) constructing a second series of I-CreI variants having at least one substitution in a second functional subdomain of the LAGLIDADG (SEQ ID NO: 229) core domain situated from positions 44 to 77 of I-CreI,
(c) selecting and/or screening the variants from the first series of (a) which can cleave a mutant I-CreI site wherein (i) the nucleotide triplet in positions −10 to −8 of the I-CreI site has been replaced with the nucleotide triplet which is present in position −10 to −8 of a genomic DNA target which is present in a xeroderma pigmentosum gene and (ii) the nucleotide triplet in positions +8 to +10 has been replaced with the reverse complementary sequence of the nucleotide triplet which is present in position −10 to −8 of said genomic target,
(d) selecting and/or screening the variants from the second series of (b) which can cleave a mutant I-CreI site wherein (i) the nucleotide triplet in positions −5 to −3 of the I-CreI site has been replaced with the nucleotide triplet which is present in position −5 to −3 of said genomic target in (c) and (ii) the nucleotide triplet in positions +3 to +5 has been replaced with the reverse complementary sequence of the nucleotide triplet which is present in position −5 to −3 of said genomic target,
(e) selecting and/or screening the variants from the first series of (a) which can cleave a mutant I-CreI site wherein (i) the nucleotide triplet in positions +8 to +10 of the I-CreI site has been replaced with the nucleotide triplet which is present in positions +8 to +10 of said genomic target in (c) and (ii) the nucleotide triplet in positions −10 to −8 has been replaced with the reverse complementary sequence of the nucleotide triplet which is present in position +8 to +10 of said genomic target,
(f) selecting and/or screening the variants from the second series of (b) which can cleave a mutant I-CreI site wherein (i) the nucleotide triplet in positions +3 to +5 of the I-CreI site has been replaced with the nucleotide triplet which is present in positions +3 to +5 of said genomic target in (c) and (ii) the nucleotide triplet in positions −5 to −3 has been replaced with the reverse complementary sequence of the nucleotide triplet which is present in position +3 to +5 of said genomic target,
(g) combining in a single variant, the mutation(s) in positions 28 to 40 and 44 to 70 of two variants from (c) and (d), to obtain a novel homodimeric I-CreI variant which cleaves a sequence wherein (i) the nucleotide triplet in positions −10 to −8 is identical to the nucleotide triplet which is present in positions −10 to −8 of said genomic target in (c), (ii) the nucleotide triplet in positions +8 to +10 is identical to the reverse complementary sequence of the nucleotide triplet which is present in positions −10 to −8 of said genomic target, (iii) the nucleotide triplet in positions −5 to −3 is identical to the nucleotide triplet which is present in positions −5 to −3 of said genomic target and (iv) the nucleotide triplet in positions +3 to +5 is identical to the reverse complementary sequence of the nucleotide triplet which is present in positions −5 to −3 of said genomic target,
(h) combining in a single variant, the mutation(s) in positions 28 to 40 and 44 to 70 of two variants from (e) and (f), to obtain a novel homodimeric I-CreI variant which cleaves a sequence wherein (i) the nucleotide triplet in positions +3 to +5 is identical to the nucleotide triplet which is present in positions +3 to +5 of said genomic target in (c), (ii) the nucleotide triplet in positions −5 to −3 is identical to the reverse complementary sequence of the nucleotide triplet which is present in positions +3 to +5 of said genomic target, (iii) the nucleotide triplet in positions +8 to +10 of the I-CreI site has been replaced with the nucleotide triplet which is present in positions +8 to +10 of said genomic target and (iv) the nucleotide triplet in positions −10 to −8 is identical to the reverse complementary sequence of the nucleotide triplet in positions +8 to +10 of said genomic target,
(i) combining the variants obtained in (g) and (h) to form heterodimers, and
(j) selecting and/or screening the heterodimers from (i) which are able to cleave said DNA target sequence from a xeroderma pigmentosum gene.

2. The variant according to claim 1, wherein said substitution(s) in the subdomain situated from positions 44 to 77 of I-CreI are in positions 44, 68, 70, 75, 77, or a combination thereof.

3. The variant according to claim 1, wherein said substitution(s) in the subdomain situated from positions 44 to 77 of I-CreI are from positions 44 to 70.

4. The variant according to claim 1, wherein said substitution(s) in the subdomain situated from positions 26 to 40 of I-CreI are in positions 28, 30, 32, 33, 38, 40, or a combination thereof.

5. The variant according to claim 1, wherein said substitution(s) in the subdomain situated from positions 26 to 40 of I-CreI are from positions 28 to 40.

6. The variant according to claim 1, wherein said substitutions are replacement of the initial amino acids with amino acids selected in the group consisting of A, D, E, G, H, K, N, P, Q, R, S, T, Y, C, W, L and V.

7. The variant according to claim 1, which comprises one or more substitutions in positions: 19, 24, 42, 69, 80, 85, 87, 109, 133 and 161 of I-CreI.

8. The variant according to claim 1, which comprises a substitution of the aspartic acid in position 75 with an uncharged amino acid.

9. The variant according to claim 8, wherein said uncharged amino acid is an asparagine or a valine residue.

10. The variant according to claim 1, which is obtained by a method comprising (a) to (j), wherein the method further comprises: random mutagenesis on at least one monomer of the heterodimer formed in (i) or obtained in (j) and selection and/or screening of the heterodimers having improved activity towards said DNA target from a xeroderma pigmentosum gene.

11. The variant according to claim 1, which is an homodimer that can cleave a palindromic or pseudo-palindromic DNA target sequence from a xeroderma pigmentosum gene.

12. The variant according to claim 1, which is an heterodimer, resulting from the association of a first and a second monomer having different mutations in positions 26 to 40 and 44 to 77 of I-CreI, said heterodimer can cleave a non-palindromic DNA target sequence from a xeroderma pigmentosum gene.

13. The variant according to claim 1, wherein said DNA target sequence is from a human xeroderma pigmentosum gene.

14. The variant according to claim 12, wherein said DNA target is a sequence from the human XPA gene, selected from the group consisting of the sequences SEQ ID NO: 45 to 57.

15. The variant according to claim 12, wherein said DNA target is a sequence from the human XPB gene, selected from the group consisting of the sequences SEQ ID NO: 58 to 86.

16. The variant according to claim 12, wherein said DNA target is a sequence from the human XPC gene, selected from the group consisting of the sequences SEQ ID NO: 1 to 24.

17. The variant according to claim 12, wherein said DNA target is a sequence from the human XPD gene, selected from the group consisting of the sequences SEQ ID NO: 87 to 119.

18. The variant according to claim 12, wherein said DNA target is a sequence from the human APE gene, selected from the group consisting of the sequences SEQ ID NO: 120 to 166.

19. The variant according to claim 12, wherein said DNA target is a sequence from the human XPF gene, selected from the group consisting of the sequences SEQ ID NO: 167 to 188.

20. The variant according to claim 12, wherein said DNA target is a sequence from the human XPG gene, selected from the group consisting of the sequences SEQ ID NO: 189 to 216.

21. The variant according to claim 14, which is a heterodimer, wherein the first and the second monomers have amino acids in positions 24, 26, 28, 30, 33, 38, 40, 42, 44, 68, 70, 75, 77, 80, or a combination thereof of I-CreI, which are as indicated in Table XXII:

| First monomer | Second monomer |
|---|---|
| 28K33S38R40D44K68R70G75N | 28R30D38R44K68A70S75N77I |
| 28K30G38H44Q68R70Q75N | 28K30G38K44Q68R70S75R77T80K |
| 28K33T38A40Q44N68K70S75R77N | 30D33R38G44N68K70S75R77N |
| 28K30G38H44R68Y70S75E77Y | 28K33N38Q40Q44K68R70E75N |
| 28K30N38Q44Q68A70N75N | 28Q33Y38R40K42R44Q70S75N77N |
| 30N33H38Q44K68R70E75N | 28K33R38A40Q44Q68R70S75N |
| 28K30N38Q44K68H70E75N | 28K33R38A40Q44Q68R70S75N |
| 28K30G38G44Q68R70G75N | 30D33R38G44Q68R70S75N |
| 28K30G38H44Q68R70S75R77T80K | 28K33T38A40Q44Q68R70G75N |
| 30N33H38Q44Q68A70N75N | 24I26Q28K30N33Y38Q40S44K68R70E75N |
| 28K30N38Q44K68A70N75N | 28K33R38A40Q44A68R70G75N |
| 28K33R38E40R44K68S70N75N | 28K30G38H44R68Y70S75E77Y |
| 28K33R38A40Q44N68R70N75N | 30D33R38G44A68N70N75N |

22. The variant according to claim 15, which is a heterodimer wherein the first and the second monomers have amino acids in positions 24, 26, 28, 30, 33, 38, 40, 42, 44, 68, 70, 75, 77, 80, or a combination thereof of I-CreI which are as indicated in Table XXIII:

| First monomer | Second monomer |
|---|---|
| 30R33G38S44K68S70N75N | 30N33H38Q44Q68R70S75R77T80K |
| 30N33H38A44K68R70E75N | 30D33R38T44K68S70N75N |
| 28K33N38Q40Q44R68R70R75N | 28K33R38A40Q44R68R70R75N |
| 28R30D38Q44E68R70A75N | 28Q33S38R40K44K68T70T75N |
| 30N33H38A44A68R70G75N | 28K33T38A40Q68K44Q68Y70S75R77Q |
| 30N33H38A44A68N70N75N | 30N33H38Q44R68Y70S75E77Y |
| 28K33S38Q40Q44R68R70R75N | 28K33R38Q40S44K68R70G75N |
| 28K33T38A40A44Q68R70S75R77T80K | 30N33H38Q44K68Y70S75Q77N |
| 30N33T38A42R44Q70S75N77N | 28R33A38Y40Q44K68R70E75N |
| 30D33R38G44K68A70N75N | 28K30G38H44R68R70R75N |
| 28R33A38Y40Q44Q68R70G75N | 30N33H38A44A68S70R75N |
| 28Q33Y38Q40K44A68N70N75N | 30D33R38G44Q68R70S75R77T80K |
| 30N33H38Q44K68Y70S75D77T | 28Q33Y38Q40K44K68Y70S75D77T |
| 28K33S38Q40Q44Q68R70S75N | 28K30G38H44K68H70E75N |
| 30N33H38A44A68R70S75N | 28K30G38H44A68N70N75N |
| 30D33R38T44K68S70N75N | 30D33R38G44R68Y70S75E77Y |
| 24I28Q28K30N33Y38Q40S44K68H70E75N | 28K30N38Q44K68Y70S75D77T |
| 28K33T38A40A44K68Y70S75Q77N | 30N33H38Q44Q68R70G75N |
| 30N33H38A44N68K70S75R77N | 28R33A38Y40Q44R68Y70S75E77Y |
| 28Q33Y38Q40K44K68T70T75N | 24I26Q28K30N33Y38Q40S44E68R70A75N |
| 24I28Q28K30N33Y38Q40S44Q68R70N75N | 30D33R38T44A68R70S75Y77Y |
| 28K33T38A40Q44Q68R70Q75N | 28K30N38Q44Q68Y70S75R77Q |
| 28K33S38R40D44Y68D70S75R77T | 28K33S38R40D44K68T70T75N |
| 30N33H38A44R68Y70S75E77Y | 28R33A38Y40Q44Q68R70S75R77T80K |
| 28Q33Y38R40K44Q68R70S75R77T80K | 30R33G38S44R68R70R75N |
| 30R33G38S44Q68R70S75N | 28R30D38Q44K68A70N75N |
| 30D33R38T42R44Q70S77N | 30N33T38A44A68R70S75N |
| 30N33T38A44Q68R70S75N | 30N33H38Q44E68R70A75N |
| 30N33H38A44K68A70N75N | 30N33T38Q44A68S70R75N |

23. The variant according to claim 16, which is a heterodimer, wherein the first and the second monomers have amino acids in positions 28, 30, 33, 38, 40, 42, 44, 68, 70, 75, 77, 80, 133, or a combination thereof of I-CreI which are as indicated in Table XXIV:

| First monomer | Second monomer |
|---|---|
| 30D33R38G44R68Y70S75Y77T | 28K33R38E40R44R68Y70S75E77V |
| 30D33R38T44T68Y70S75R77T | 28K33R38N40Q44N68R70N75N |
| 28K33R38E40R44R68Y70S75E77I | 28Q33Y38R40K42R44Q70S77N |
| 28Q33S38R40K44Q68Y70S75N77Y | 28T33T38Q40R44T68E70S75R77R |
| 30D33R38T44N68R70S75Q77R | 28R33A38Y40Q44D68Y70S75S77R |
| 28K33R38Q40A44T68R70S75Y77T133V | 28K33R38E40R44K68Y70S75D77T |
| 28K33N38Q40Q44R68Y70S75E77I | 28K33T38A40Q44K68Q70S75N77R |
| 28E33R38R40K44Q68Y70S75N77Y | 28Q33S38R40K44A68R70S75R77L |
| 28K33T38A40Q44A68R70S75R77L | 28A33T38Q40R44R68S70S75E77R |
| 28K30N38Q44Q68R70S75R77T80K | 28T33T38Q40R44T68Y70S75R77V |
| 28K30N38Q44A68Y70S75Y77K | 28K33R38E40R44T68R70S75Y77T133V |
| 28K33R38Q40A44Q68R70N75N | 28K33R38A40Q44Q68R70S75N77K |
| 30N33H38Q44K68A70S75N77I | 28K33N38Q40Q44R68Y70S75E77V |
| 28Q33S38R40K44N68R70S75R77D | 28T33R38Q40R44Q68R70S75R77T80K |
| 28Q33R38R40K44T68Y70S75R77T | 28Q33Y38R40K44Y68D70S75R77V |
| 28K33T38A40A44T68E70S75R77R | 28K30N38Q44A68N70S75Y77R |
| 28Q33Y38Q40K44Q68R70S75R77T80K | 28K33R38Q40A44Q68R70N75N |
| 28K33R38A40Q44K68A70S75N77I | 28K33R38E40R44R68Y70S75Y77T |
| 28T33T38Q40R44Q68R70S75D77K | 28E33R38R40K44Q68R70S75R77T80K |
| 28R33A38Y40Q44Q68R70S75R77T80K | 28Q33Y38R40K44T68Y70S75R77T |
| 28Q33S38R40K42T44K70S75N77Y | 28R33A38Y40Q44A68R70S75E77R |
| 30D33R38T44A68Y70S75Y77K | 28Q33Y38R40K42R44Q70S75N77N |
| 28K33R38E40R44K68Q70S75N77R | 28Q33S38R40K44R68Y70S75E77V |
| 28K30N33H38Q40S44Q68R70R75N | 28K30N33R38A40Q44K68R70N75N |

24. The variant according to claim 17, which is a heterodimer wherein the first and the second monomers have amino acids in positions 28, 30, 33, 38, 40, 44, 68, 70, 75, 77, 80, or a combination thereof of I-CreI which are as indicated in Table XXV:

| First monomer | Second monomer |
|---|---|
| 30A33D38H44K68R70E75N | 28K30G38K44K68S70N75N |
| 30N33T38Q44Q68R70G75N | 30N33H38Q44A68R70S75E77R |
| 30N33H38A44N68R70N75N | 30N33H38Q44Q68R70S75R77T80K |
| 28K33R38E40R44E68R70A75N | 28R33A38Y40Q44A68S70R75N |
| 28K33T38R40Q44K68R70E75N | 28Q33S38R40K44K68R70G75N |
| 30D33R38T44K68R70E75N | 28K30G38G44Q68Y70S75R77Q |
| 30D33R38T44K68R70E75N | 30R33G38S44A68R70S75N |
| 28K30G38H44A68R70N75N | 28K33S38R40D44A68N70N75N |
| 30N33H38Q44Y68D70S75R77T | 28R30D38R44A68N70N75N |
| 28K33R38Q40S44K68T70T75N | 30D33R38G44K68R70E75N |
| 28R33A38Y40Q44Q68R70Q75N | 28K30G38H44R68Y70S75E77Y |
| 33R38E44Q68R70G75N | 28K33R38E40R44K68R70E75N |
| 30R33G38S44K68R70E75N | 30D33R38G44A68R70N75N |
| 30R33G38S44A68R70S75N | 30D33R38G44K68R70G75N |
| 30N33H38A44K68R70G75N | 28Q33Y38R40K44K68A70N75N |
| 30N33T38A44N68R70N75N | 28K30G38K44Q68R70S75N |
| 28R33A38Y40Q44K68R70G75N | 28Q33Y38R40K44T68Y70S75R77V |
| 28Q33Y38R40K44K68Y70S75Q77N | 33T44Q68R70S75N |
| 28K30G38G44A68N70N75N | 28K33R38E40R44E68R70A75N |
| 28K33R38A40Q44R68R70R75N | 28K33R38E40R44T68Y70S75R77V |
| 28K33R38Q40S44K68R70E75N | 30R33G38S44A68R70S75E77R |
| 28Q33Y38R40K44D68R70N75N | 28K33R38A40Q44R68R70R75N |
| 30N33H38A44Q68R70G75N | 28Q33Y38R40K44A68S70R75N |
| 30D33R38T44A68R70N75N | 28K33R38E40R44A68R70S75N |
| 28Q33Y38R40K44E68R70A75N | 33R38E44N68R70N75N |
| 28K30G38H44A68R70S75N | 30N33T38Q44R68Y70S75E77Y |
| 28K33R38E40R44Q68R70G75N | 30N33T38A44R68R70R75N |
| 28Q33Y38Q40K44Q68R70S75R77T80K | 30R33G38S44G68Q70T75N |
| 30N33H38Q44D68R70N75N | 28K30G38K44G68Q70T75N |
| 28K33N38Q40Q44A68N70N75N | 28K30G38H44Q68R70G75N |
| 28K33R38E40R44K68R70E75N | 28K30G38H44A68R70S75N |
| 28Q33S38R40K44K68R70E75N | 28K33T38A40A44A68R70S75N |
| 28K33N38Q40Q44K68T70T75N | 28K30G38H44Q68A70N75N |

25. The variant according to claim 18, which is a heterodimer wherein the first and the second monomers have amino acids in positions 28, 30, 33, 38, 40, 44, 68, 70, 75, 77, 80, 133, or a combination thereof of I-CreI which are as indicated in Table XXVI:

| First monomer | Second monomer |
|---|---|
| 28K30N38Q44K68R70E75N | 28Q33S38R40K44A68S70R75N |
| 28K30G38H44Q68R70N75N | 28K30G38K44Y68D70S75R77T |
| 28K30N38Q44D68Y70S75S77R | 28R33A38Y40Q44R68Y70S75E77Y |
| 30N33H38Q44Q68R70S75N | 30N33H38A44A68R70S75N |
| 28Q33S38R40K44K68T70G75N | 28K33R38Q40S44A68R70S75E77R |
| 28K30G38H44K68R70E75N | 30N33H38A44K68R70G75N |
| 28R33A38Y40Q44D68R70R75N | 28K33N38Q40Q44Q68R70S75R77T80K |
| 28K33R38E40R44R68R70R75N | 30Q33G38H44A68R70G75N |
| 30D33R38T44K68R70E75N | 28Q33Y38R40K44A68R70S75N |
| 44Q68R70Q75N | 33T44Q68Y70S75R77Q |
| 28K33T38R40Q44A68R70G75N | 28K33R38E40R44Q68R70S75R77T80K |
| 30D33R38G44K68S70N75N | 30D33R38T44A68S70R75N |
| 28Q33Y38Q40K44K68R70E75N | 28K30G38H44A68S70R75N |
| 30N33H38A44D68Y70S75S77R | 28K33R38E40R44Q68R70S75R77T80K |
| 28K33S38Q40Q44T68R70S75Y77T133V | 30N33T38Q44Q68R70S75N |
| 30D33R38T44K68R70E75N | 28Q33Y38R40K44A68S70R75N |
| 30D33R38T44Q68R70S75N | 28K33R38Q40S44K68Y70S75D77T |
| 28R33A38Y40Q44K68T70G75N | 44N68R70R75N |
| 28K33R38E40R44Q68R70G75N | 28K33T38R40Q44Q68Y70S75R77Q |
| 30N33H38Q44R68Y70S75E77Y | 28K33R38Q40S70S75N |
| 28R33A38Y40Q44T68Y70S75R77V | 30R33G38S44A68R70S75Y77Y |
| 28K33R38A40Q44K68R70E75N | 28K30G38H44N68R70R75N |
| 28K33R38Q40S44D68R70N75N | 28K33S38R40D44A68R70G75N |
| 30D33R38G44K68H70E75N | 28K30G38G44A68R70G75N |
| 30N33H38Q44R68R70R75N | 30N33T38A44K68R70E75N |
| 30D33R38T44K68T70G75N | 30R33G38S44Q68R70N75N |
| 28K30G38H44R68Y70S75E77Y | 28R33A38Y40Q44Q68R70S75R77T80K |
| 28R33A38Y40Q44A68R70S75N | 28K30G38H44Q68R70S75R77T80K |
| 30N33H38A44K68A70N75N | 28Q33Y38Q40K44Y68D70S75R77T |
| 28Q33Y38R40K44K68R70E75N | 28K33R38E40R44N68R70R75N |
| 28K33R38A40Q44K68Y70S75Q77N | 28K33S38Q40Q44K68R70E75N |
| 28Q33Y38R40K44D68R70R75N | 30N33H38A44A68R70N75N |
| 30D33R38G44A68N70N75N | 30D33R38T44K68R70E75N |
| 28K33N38Q40Q44D68R70N75N | 30D33R38T44A68R70S75N |
| 30N33H38A44Q68R70S75N | 30Q33G38H44A68N70N75N |
| 28R33A38Y40Q44K68R70E75N | 28K33R38Q40S44E68R70A75N |
| 28R30D38Q44K68R70E75N | 28K33S38R40D70S75N |
| 30A33D38H44K68H70E75N | 28K33T38A40A44N68R70R75N |
| 28K30N38Q44K68A70S75N77I | 28K33R38E40R44A68S70R75N |
| 28Q33Y38Q40K44K68R70E75N | 28Q33Y38Q40K44Q68R70S75R77T80K |
| 30D33R38G44N68R70A75N | 28Q33Y38Q40K44Q68R70S75R77T80K |
| 30N33H38Q44R68R70R75N | 28R33A38Y40Q44A68R70S75N |
| 28K33S38Q40Q44R68Y70S75E77I | 30N33T38A44A68R70S75N |
| 28K30G38K44N68R70N75N | 28K33R38E40R44D68Y70S75S77R |
| 28K33R38A40Q44K68R70E75N | 28K30N38Q44Q68Y70S75R77Q |
| 28K33T38R40Q44K68R70G75N | 28R33A38Y40Q44E68R70A75N |
| 28K33N38Q40Q44Q68Y70S75R77Q | 30D33R38T44Q68R70G75N |

26. The variant according to claim 19, which is a heterodimer wherein the first and the second monomers have amino acids in positions 28, 30, 33, 38, 40, 44, 68, 70, 75, 77, 80, or a combination thereof of I-CreI which are as indicated in Table XXVII:

| First monomer | Second monomer |
|---|---|
| 30N33H38Q44T68Y70S75R77V | 30D33R38T44Q68R70G75N |
| 28K33S38R40A44K68S70N75N | 28E33R38R40K44Q68R70G75N |
| 28K33T38A40A44Q68R70N75N | 30D33R38G44Q68R70S75N |
| 28Q33S38R40K44R68Y70S75E77Y | 28K33T38A40Q44T68Y70S75R77T |
| 28R30D38Q44Q68R70G75N | 28R33A38Y40Q44Q68R70S75R77T80K |
| 28K30N38Q44A68R70S75Y77Y | 28K33T38R40Q44A68R70S75Y77Y |
| 28K33T38R40Q44Q68Y70S75R77Q | 28K30N38Q44K68Y70S75Q77N |
| 28K33T38A40Q44Q68R70S75N | 28R30D38Q44A68N70S75Y77R |
| 28K33S38Q40Q44Q68R70S75N | 28K33N38Q40Q44A68R70S75Y77Y |
| 28K33R38A40Q44E68R70A75N | 28K33R38A40Q44Q68R70S75R77T80K |
| 28K33T38A40A42T44K70S75N77Y | 28K33T38A40A44T68Y70S75R77V |
| 30N33T38Q44K68R70E75N | 28T33R38Q40R44Q68R70N75N |
| 28Q33Y38Q40K44Q68R70G75N | 28R30D38Q44T68Y70S75R77V |
| 28K33S38Q40Q44Q68R70S75N | 28K30N38Q44K68R70G75N |
| 28K33T38A40Q44Q68R70G75N | 28Q33Y38R40K44Q68R70R75E77R |
| 28Q33Y38R40K44Q68R70S75R77T80K | 28A33T38Q40R44Q68R70S75R77T80K |
| 30D33R38T44A68R70G75N | 28K33R38E40R44Q68R70G75N |
| 28Q33S38R40K44R68Y70S75D77N | 30D33R38T44K68R70E75N |
| 28K30G38G44T68Y70S75R77V | 28R33A38Y40Q44K68A70S75N77I |
| 28Q33Y38R40K44D68Y70S75S77R | 30D33R38T44E68R70A75N |
| 30N33T38A44Q68R70G75N | 28K30G38H44R68Y70S75E77Y |
| 28K30G38H44A68N70N75N | 30N33H38A44R68Y70S75E77V |

27. The variant according to claim 20, which is a heterodimer wherein the first and the second monomers have amino acids in positions 28, 30, 33, 38, 40, 42, 44, 68, 70, 75, 77, 80, 133, or a combination thereof of I-CreI which are as indicated in Table XXVIII:

| First monomer | Second monomer |
|---|---|
| 30D33R38T44K68R70E75N | 30D33R38T44Q68N70R75N |
| 30D33R38T44E68R70A75N | 28T33T38Q40R44Q68Y70S75R77Q |
| 30N33Y38Q44Q68R70S75R77T80K | 28K33T38A40A44K68R70E75N |
| 28T33R38S40R44A68R70S75N | 30N33H38Q44N68R70S75R77D |
| 28K30G38H44Q68R70Q75N | 28T33R38Q40R44A68R70S75N |
| 28K33T38R40Q44N68R70A75N | 28T33R38Q40R44K68R70E75N |
| 28K30G38H44A68Q70N75N | 32T33C44Y68D70S75R77T |
| 28R30D38Q44T68R70S75Y77T133V | 30N33T38A44Q68R70S75N |
| 28Q33Y38Q40K44A68R70N75N | 28K30N38Q44Q68R70G75N |
| 28K33R38E40R44Q68R70S75N | 28K33R38Q40A44K68Q70S75N77R |
| 30A33D38H44K68G70T75N | 28K33T38A40Q44N68R70S75R77D |
| 32T33C44N68R70A75N | 28R33A38Y40Q44K68R70E75N |
| 28K30G38H44A68R70D75N | 30N33H38A44Q68R70S75N |
| 30R33G38S44K68T70S75N | 28R33A38Y40Q42T44K70S75N77Y |
| 28R33S38Y40Q44K68T70S75N | 28R33A38Y40Q44N68R70S75R77D |
| 30N33H38Q44Q68R70D75N | 28Q33S38R40K44K68H70E75N |
| 28R33S38Y40Q44Y68E70S75R77V | 28K30N38Q44K68R70E75N |
| 32T33C44A68R70S75N | 28T33T38Q40R44T68Y70S75R77T |
| 30N33H38A44D68Y70S75S77R | 28K33R38E40R44T68Y70S75R77T |
| 28R33A38Y40Q44N68R70N75N | 28R33A38Y40Q44Q68Y70S75N77Y |
| 30N33Y38Q44Q68S70K75N | 30D33R38G44A68N70S75Y77R |
| 28K33T38A40Q44E68R70A75N | 28R33A38Y40Q44S68Y70S75Y77V |
| 28A33T38Q40R44E68R70A75N | 28K30G38G44A68N70N75N |
| 28K33R38A40Q44N68R70N75N | 28K33S38R40D42T44K70S75N77Y |
| 32T33C44Q68R70D75N | 28K33T38A40A44A68R70N75N |
| 32T33C44K68A70S75N | 30D33R38G44N68R70N75N |
| 28Q33Y38R40K44A68R70S75N | 30D33R38T42T44K70S75N77Y |
| 30D33R38T44K68G70T75N | 30D33R38G44A68D70K75N |

28. The variant according to claim 12, wherein the first monomer has amino acids in positions 19, 28, 30, 33, 38, 40, 69, 70, 75, 87, or a combination thereof of I-CreI which are selected from the group consisting of: 28K30N33S38R40S70S75N, 28A30N33S38R40K70S75N, 19A28A30N33S38R40K70S75N, 19A28A30N33Y38R40K70S75N87L, and 19A28A30N33S38R40K69G70S75N, and the second monomer has amino acids in positions 28, 30, 33, 38, 40, 44, 68, 70, 75, 85, 109, 161, or a combination thereof of I-CreI which are a selected from the group consisting of: 28E30N33Y38R40K44K68S70S75N, 28K30G33Y38R40S44K68R70E75N, 28E30N33Y38R40K44K68R70E75N85R109T, 28E30N33Y38R40K44K68R70E75N85R109T161F 28E30N32R33Y38Q40K44K68R70E75N85R109T, 28S30N33Y38R40K44K68S70S75N, 28S30N33Y38R40K44K68R70D75N, 28S30N33Y38R40K44K68A70S75N, 28K30G33Y38H40S44K68R70E75N, 28K30G33Y38H40S44K68A70G75N, 28K30G33Y38R40S44K68R70E75N, 28K30G33Y38R40S44K68T70H75N, 28K30G33Y38R40S44K68S70S75N, and 28K30G33Y38R40S44K68T70S75N.

29. A single-chain chimeric meganuclease comprising two monomers or core domains of one or two I-CreI variants of claim 1, or a combination of both.

30. A polynucleotide fragment encoding a variant of claim 1.

31. An expression vector comprising at least one polynucleotide fragment of claim 30.

32. The expression vector according to claim 31, which comprises two different polynucleotide fragments, each encoding one of the monomers of an heterodimeric variant, said heterodimeric variant resulting from the association of a first and a second monomer having different mutations in positions 26 to 40 and 44 to 77 of I-CreI, said heterodimer which can cleave a non-palindromic DNA target sequence from a xeroderma pigmentosum gene.

33. A vector, which includes a targeting construct comprising a sequence to be introduced flanked by sequences sharing homologies with the regions surrounding the genomic DNA cleavage site of the variant as defined in claim 1.

34. The vector according to claim 31, which comprises a targeting construct comprising a sequence to be introduced flanked by sequences sharing homologies with the regions surrounding the genomic DNA cleavage site of the variant.

35. The vector according to claim 33, wherein said sequence to be introduced is a sequence which repairs a mutation in a xeroderma pigmentosum gene.

36. The vector according to claim 35, wherein the sequence which repairs said mutation is the correct sequence of said xeroderma pigmentosum gene.

37. The vector according to claim 35, wherein the sequence which repairs said mutation comprises the exons of said xeroderma pigmentosum gene downstream of the genomic cleavage site of the variant, fused in frame, and a polyadenylation site to stop transcription in 3′.

38. The vector according to claim 33, wherein said targeting construct comprises a sequence of the XPA gene which can repair a cleavage in exons 1 to 6 of the XPA gene and is selected from the group consisting of: positions 34 to 233, 201 to 400, 3493 to 3692, 7627 to 7826, 9994 to 10193, 10151 to 10350 12513 to 12712, 12531 to 12730, 21679 to 21878, 21844 to 22043, 21955 to 22154, 22228 to 22427 and 22234 to 22433.

39. The vector according to claim 33, wherein said targeting construct comprises a sequence of the XPB gene which can repair a cleavage in exons 1 to 15 of the XPB gene and is selected from the group consisting of positions: −40 to 159, 357 to 556, 1335 to 1534, 1336 to 1535, 1457 to 1656, 3624 to 3823, 4108 to 4307, 5015 to 5214, 5148 to 5347, 5284 to 5483, 5420 to 5619, 7377 to 7576, 13517 to 13716, 13552 to 13751, 13633 to 13832, 14697 to 14896, 14760 to 14959, 14881 to 15080, 21139 to 21338, 21207 to 21406, 22796 to 22995, 32378 to 32577, 33003 to 33202, 34481 to 34680, 34869 to 35068, 34891 to 35090, 36584 to 36783, 36634 to 36833, and 36639 to 36838.

40. The vector according to claim 33, wherein said targeting construct comprises a sequence of the XPC gene which can repair a cleavage in exons 1 to 16 of the XPC gene and is selected from the group consisting of positions: 105 to 304, 5704 to 5903, 7973 to 8172, 9887 to 10086, 10173 to 10372, 11263 to 11462, 13051 to 13250, 13432 to 13631, 18619 to 18818, 19580 to 19779, 20303 to 20502, 20349 to 20548, 20389 to 20588, 21985 to 22184, 21990 to 22189, 22028 to 22227, 22102 to 22301, 26017 to 26216, 29566 to 29765, 29726 to 29925, 30416 to 30615, 31166 to 31365 and 32317 to 32516.

41. The vector according to claim 33, wherein said targeting construct comprises a sequence of the XPD gene which can repair a cleavage in exons 1 to 23 of the XPD gene and is selected from the group consisting of positions: −87 to 112, 812 to 1011, 1319 to 1518, 1324 to 1523, 1426 to 1625, 1717 to 1916, 1867 to 2066, 5473 to 5672, 5585 to 5784, 5637 to 5836, 5920 to 6119, 6050 to 6249, 6290 to 6489, 6392 to 6591, 6472 to 6671, 6581 to 6780, 8830 to 9029, 8943 to 9142, 12661 to 12860, 12991 to 13190, 13084 to 13283, 14614 to 14813, 14817 to 15016, 15528 to 15727, 15878 to 16077, 15936 to 16135, 17023 to 17222, 17350 to 17549, 17365 to 17564, 17572 to 17771, 18347 to 18546, 18370 to 18569, and 18641 to 18840.

42. The vector according to claim 33, wherein said targeting construct comprises a sequence of the APE gene which can repair a cleavage in exons 1 to 27 of the APE gene and is selected from the group consisting of positions: 10-209, 1295-1494, 2899-3098, 3488-3687, 3616-3815, 6093-6292, 6194-6393, 7034-7233, 7653-7852, 8753-8952, 9781-9980, 9966-10165, 10511-10710, 10665-10864, 11534-11733, 16439-16638, 16667-16866, 18268-18647, 18757-18956, 18863-19062, 19179-19378, 19266-19465, 19596-19795, 20714-20913, 20938-21137, 21099-21298, 22568-22767, 22732-22931, 23173-23372, 23181-23380, 23954-24153, 24000-24199, 29205-29404, 29651-29850, 30280-30479, 30355-30554, 30661-30860, 30685-30884, 32150-32349, 32753-32592, 32770-32969, 32811-33010, 32836-33035, 32841-33040, 33230-33429, 33369-33568, and 33512-33711.

43. The vector according to claim 33, wherein said targeting construct comprises a sequence of the XPF gene which can repair a cleavage in exons 1 to 21 of the XPF gene and is selected from the group consisting of positions: 244-443, 1731-1930, 6429-6628, 6486-6685, 7918-8117, 10500-10699, 10676-10875, 11885-12084, 11886-12085, 12176-12375, 14073-14272, 14110-14309, 15336-15535, 15431-15630, 15574-15773, 17673-17872, 17677-17876, 24486, 24685, 27496-27695, 27822-28021, 27827-28026, and 27963-28162.

44. The vector according to claim 33, wherein said targeting construct comprises a sequence of the XPG gene which can repair a cleavage in exons 1 to 15 of the XPG gene and is selected from the group consisting of positions: 158-357, 5956-6155, 7961-7890, 8044-8243, 9977-10176, 12201-12400, 12289-12488, 15230-15429, 15803-16002, 16041-16240, 16146-16345, 16174-16373, 16174-16373, 16394-16593, 16553-16732, 16887-17086, 19487-19686, 19727-19926, 19782-19981, 20207-20406, 20500-20699, 21890-22089, 22117-22316, 25876-26075, 26450-26649, 26832-27031, 27258-27457, 29180-29379, and 29456-29655.

45. The vector according to claim 33, wherein the sequence which repairs said mutation is flanked by a sequence of the XPA gene which can repair a cleavage in exons 1 to 6 of the XPA gene and is selected from the group consisting of: positions 34 to 233, 201 to 400, 3493 to 3692, 7627 to 7826, 9994 to 10193, 10151 to 10350 12513 to 12712, 12531 to 12730, 21679 to 21878, 21844 to 22043, 21955 to 22154, 22228 to 22427 and 22234 to 22433.

46. A composition comprising at least one variant according to claim 1.

47. The composition according to claim 46, which comprises a targeting DNA construct comprising a sequence which repairs a mutation in the XP gene, flanked by sequences sharing homologies with the region surrounding the genomic DNA target cleavage site of said variant.

48. The composition according to claim 47, wherein said targeting DNA construct is included in a recombinant vector.

49. A product comprising the vector according to claim 31 and a vector which comprises a targeting construct comprising a sequence to be introduced flanked by sequences sharing homologies with the regions surrounding the genomic DNA cleavage site of the variant, as a combined preparation for simultaneous, separate or sequential use in Xeroderma pigmentosum.

50. The use of at least one variant according to claim 1 for the preparation of a medicament for preventing, improving or curing a disease associated with Xeroderma pigmentosum in an individual in need thereof.

51. A host cell which is modified by a polynucleotide according to claim 30.

52. A non-human transgenic animal comprising one or two polynucleotide fragments as defined in claim 30.

53. A transgenic plant comprising one or two polynucleotide fragments as defined in claim 30.

54. Use of at least one variant according to claim 1 for genome engineering, for non-therapeutic purposes.

55. The use according to claim 54, wherein said variant, single-chain chimeric meganuclease, vector is associated with a targeting DNA construct comprising a sequence to be introduced flanked by sequences sharing homologies with the regions surrounding the genomic DNA cleavage site of the variant.

56. A polynucleotide fragment encoding a single-chain chimeric meganuclease of claim 29.

57. An expression vector comprising at least one polynucleotide fragment of claim 56.

58. A composition comprising at least one single-chain chimeric meganuclease according to claim 29.

59. A composition comprising at least one expression vector according to claim 32.

60. The vector according to claim 32, which comprises a targeting construct comprising a sequence to be introduced flanked by sequences sharing homologies with the regions surrounding the genomic DNA cleavage site of the variant.

61. The vector according to claim 60, wherein said sequence to be introduced is a sequence which repairs a mutation in a xeroderma pigmentosum gene.

62. The vector according to claim 61, wherein the sequence which repairs said mutation is the correct sequence of said xeroderma pigmentosum gene.

63. The vector according to claim 61, wherein the sequence which repairs said mutation comprises the exons of said xeroderma pigmentosum gene downstream of the genomic cleavage site of the variant, fused in frame, and a polyadenylation site to stop transcription in 3′.

64. A host cell which is modified by a vector according to claim 31.

65. A non-human transgenic animal comprising one or two polynucleotide fragments as defined in claim 32.

66. A transgenic plant comprising one or two polynucleotide fragments as defined in claim 32.

67. The vector according to claim 33, wherein the sequence which repairs said mutation is flanked by a sequence of the XPB gene which can repair a cleavage in exons 1 to 15 of the APB gene and is selected from the group consisting of positions: −40 to 159, 357 to 556, 1335 to 1534, 1336 to 1535, 1457 to 1656, 3624 to 3823, 4108 to 4307, 5015 to 5214, 5148 to 5347, 5284 to 5483, 5420 to 5619, 7377 to 7576, 13517 to 13716, 13552 to 13751, 13633 to 13832, 14697 to 14896, 14760 to 14959, 14881 to 15080, 21139 to 21338, 21207 to 21406, 22796 to 22995, 32378 to 32577, 33003 to 33202, 34481 to 34680, 34869 to 35068, 34891 to 35090, 36584 to 36783, 36634 to 36833, and 36639 to 36838.

68. The vector according to claim 33, wherein the sequence which repairs said mutation is flanked by a sequence of the XPC gene which can repair a cleavage in exons 1 to 16 of the XPC gene and is selected from the group consisting of positions: 105 to 304, 5704 to 5903, 7973 to 8172, 9887 to 10086, 10173 to 10372, 11263 to 11462, 13051 to 13250, 13432 to 13631, 18619 to 18818, 19580 to 19779, 20303 to 20502, 20349 to 20548, 20389 to 20588, 21985 to 22184, 21990 to 22189, 22028 to 22227, 22102 to 22301, 26017 to 26216, 29566 to 29765, 29726 to 29925, 30416 to 30615, 31166 to 31365 and 32317 to 32516.

69. The vector according to claim 33, wherein the sequence which repairs said mutation is flanked by a sequence of the XPD gene which can repair a cleavage in exons 1 to 23 of the APD gene and is selected from the group consisting of positions: −87 to 112, 812 to 1011, 1319 to 1518, 1324 to 1523, 1426 to 1625, 1717 to 1916, 1867 to 2066, 5473 to 5672, 5585 to 5784, 5637 to 5836, 5920 to 6119, 6050 to 6249, 6290 to 6489, 6392 to 6591, 6472 to 6671, 6581 to 6780, 8830 to 9029, 8943 to 9142, 12661 to 12860, 12991 to 13190, 13084 to 13283, 14614 to 14813, 14817 to 15016, 15528 to 15727, 15878 to 16077, 15936 to 16135, 17023 to 17222, 17350 to 17549, 17365 to 17564, 17572 to 17771, 18347 to 18546, 18370 to 18569, and 18641 to 18840.

70. The vector according to claim 33, wherein the sequence which repairs said mutation is flanked by a sequence of the XPE gene which can repair a cleavage in exons 1 to 27 of the APE gene and is selected from the group consisting of positions: 10-209, 1295-1494, 2899-3098, 3488-3687, 3616-3815, 6093-6292, 6194-6393, 7034-7233, 7653-7852, 8753-8952, 9781-9980, 9966-10165, 10511-10710, 10665-10864, 11534-11733, 16439-16638, 16667-16866, 18268-18647, 18757-18956, 18863-19062, 19179-19378, 19266-19465, 19596-19795, 20714-20913, 20938-21137, 21099-21298, 22568-22767, 22732-22931, 23173-23372, 23181-23380, 23954-24153, 24000-24199, 29205-29404, 29651-29850, 30280-30479, 30355-30554, 30661-30860, 30685-30884, 32150-32349, 32753-32592, 32770-32969, 32811-33010, 32836-33035, 32841-33040, 33230-33429, 33369-33568, and 33512-33711.

71. The vector according to claim 33, wherein the sequence which repairs said mutation is flanked by a sequence of the XPF gene which can repair a cleavage in exons 1 to 21 of the XPF gene and is selected from the group consisting of positions: 244-443, 1731-1930, 6429-6628, 6486-6685, 7918-8117, 10500-10699, 10676-10875, 11885-12084, 11886-12085, 12176-12375, 14073-14272, 14110-14309, 15336-15535, 15431-15630, 15574-15773, 17673-17872, 17677-17876, 24486, 24685, 27496-27695, 27822-28021, 27827-28026, and 27963-28162.

72. The vector according to claim 33, wherein the sequence which repairs said mutation is flanked by a sequence of the XPG gene which can repair a cleavage in exons 1 to 15 of the XPG gene and is selected from the group consisting of positions: 158-357, 5956-6155, 7961-7890, 8044-8243, 9977-10176, 12201-12400, 12289-12488, 15230-15429, 15803-16002, 16041-16240, 16146-16345, 16174-16373, 16174-16373, 16394-16593, 16553-16732, 16887-17086, 19487-19686, 19727-19926, 19782-19981, 20207-20406, 20500-20699, 21890-22089, 22117-22316, 25876-26075, 26450-26649, 26832-27031, 27258-27457, 29180-29379, and 29456-29655.

73. The variant according to claim 2, wherein said substitution(s) in the subdomain situated from positions 44 to 77 of I-CreI are from positions 44 to 70.

74. The variant according to claim 4, wherein said substitution(s) in the subdomain situated from positions 26 to 40 of I-CreI are from positions 28 to 40.


SEQUENCE LISTING (234 sequences)

SEQ ID NO: 1 - DNA, 22 bp, Artificial Sequence
  Description of Artificial Sequence Synthetic oligonucleotide
ccagacgcggaggcctggtgga

SEQ ID NO: 2 - DNA, 22 bp, Artificial Sequence
  Description of Artificial Sequence Synthetic oligonucleotide
tcttacttgtcactggggtcca

SEQ ID NO: 3 - DNA, 22 bp, Artificial Sequence
  Description of Artificial Sequence Synthetic oligonucleotide
caccatctgaagagaggggcta

SEQ ID NO: 4 - DNA, 22 bp, Artificial Sequence
  Description of Artificial Sequence Synthetic oligonucleotide
ctgtctgggttgtgtgggttaa

SEQ ID NO: 5 - DNA, 22 bp, Artificial Sequence
  Description of Artificial Sequence Synthetic oligonucleotide
tctgcctgtgaagccagtggag

SEQ ID NO: 6 - DNA, 22 bp, Artificial Sequence
  Description of Artificial Sequence Synthetic oligonucleotide
tgagacatatcttcggagggcg

SEQ ID NO: 7 - DNA, 22 bp, Artificial Sequence
  Description of Artificial Sequence Synthetic oligonucleotide
tcaaacctggtgaagtggtaag

SEQ ID NO: 8 - DNA, 22 bp, Artificial Sequence
  Description of Artificial Sequence Synthetic oligonucleotide
cgggctggggaaagtaggacag

SEQ ID NO: 9 - DNA, 22 bp, Artificial Sequence
  Description of Artificial Sequence Synthetic oligonucleotide
tttaattactttcttaggataa

SEQ ID NO: 10 - DNA, 22 bp, Artificial Sequence
  Description of Artificial Sequence Synthetic oligonucleotide
caaaatttcttattctgtttaa

SEQ ID NO: 11 - DNA, 22 bp, Artificial Sequence
  Description of Artificial Sequence Synthetic oligonucleotide
caagcccatgacctatgtggtg

SEQ ID NO: 12 - DNA, 22 bp, Artificial Sequence
  Description of Artificial Sequence Synthetic oligonucleotide
cgagatgtcacacagaggtacg

SEQ ID NO: 13 - DNA, 22 bp, Artificial Sequence
  Description of Artificial Sequence Synthetic oligonucleotide
tgacccgcaagtgccgggttga

SEQ ID NO: 14 - DNA, 22 bp, Artificial Sequence
  Description of Artificial Sequence Synthetic oligonucleotide
ctgtctggcctttgcagtttca

SEQ ID NO: 15 - DNA, 22 bp, Artificial Sequence
  Description of Artificial Sequence Synthetic oligonucleotide
tggcctttgcagtttcaggcta

SEQ ID NO: 16 - DNA, 22 bp, Artificial Sequence
  Description of Artificial Sequence Synthetic oligonucleotide
tttgcccactgccattggctta

SEQ ID NO: 17 - DNA, 22 bp, Artificial Sequence
  Description of Artificial Sequence Synthetic oligonucleotide
ccatctatcccgagacagctcg

SEQ ID NO: 18 - DNA, 22 bp, Artificial Sequence
  Description of Artificial Sequence Synthetic oligonucleotide
tgtcccgcattcccgcagtggg

SEQ ID NO: 19 - DNA, 22 bp, Artificial Sequence
  Description of Artificial Sequence Synthetic oligonucleotide
ctaaccgtgctcggaaagcccg

SEQ ID NO: 20 - DNA, 22 bp, Artificial Sequence
  Description of Artificial Sequence Synthetic oligonucleotide
ttcccctgctggccaaatgctg

SEQ ID NO: 21 - DNA, 22 bp, Artificial Sequence
  Description of Artificial Sequence Synthetic oligonucleotide
ctgtcttccacaaactggggag

SEQ ID NO: 22 - DNA, 22 bp, Artificial Sequence
  Description of Artificial Sequence Synthetic oligonucleotide
tctgctcatcagggagaggctg

SEQ ID NO: 23 - DNA, 22 bp, Artificial Sequence
  Description of Artificial Sequence Synthetic oligonucleotide
cccaccactgccacctgtccag

SEQ ID NO: 24 - DNA, 22 bp, Artificial Sequence
  Description of Artificial Sequence Synthetic oligonucleotide
ctgccttttttgaaggatccta

SEQ ID NO: 25 - DNA, 24 bp, Artificial Sequence
  Description of Artificial Sequence Synthetic oligonucleotide
tcaaaacgtcgtacgacgttttga

SEQ ID NO: 26 - DNA, 501 bp, Artificial Sequence
  Description of Artificial Sequence Synthetic polynucleotide
atggccaataccaaatataacaaagagttcctgctgtacctggccggctttgtggacggtgacggtagcatcatcgctcagattaaaccaaaccagtcttataagtttaaacatcagctaagcttgacctttcaggtgactcaaaagacccagcgccgttggtttctggacaaactagtggatgaaattggcgttggttacgtacgtgatcgcggatccgtttccaactacatcttaagcgaaatcaagccgctgcacaacttcctgactcaactgcagccgtttctgaaactgaaacagaaacaggcaaacctggttctgaaaattatcgaacagctgccgtctgcaaaagaatccccggacaaattcctggaagtttgtacctgggtggatcagattgcagctctgaacgattctaagacgcgtaaaaccacttctgaaaccgttcgtgctgtgctggacagcctgagcgagaagaagaaatcctccccggcggccgac

SEQ ID NO: 27 - DNA, 68 bp, Artificial Sequence
  Description of Artificial Sequence Synthetic primer
ctgaaaggtcaagcttagmbbatgtttaaacttmbbagactgmbbtggtttaatctgagcgatgatgc

SEQ ID NO: 28 - DNA, 68 bp, Artificial Sequence
  Description of Artificial Sequence Synthetic primer
ctgaaaggtcaagcttagmbbatgtttaaacttataagactgmbbtggmbbaatctgagcgatgatgc

SEQ ID NO: 29 - DNA, 18 bp, Artificial Sequence
  Description of Artificial Sequence Synthetic primer
ctaagcttgacctttcag

SEQ ID NO: 30 - DNA, 24 bp, Artificial Sequence
  Description of Artificial Sequence Synthetic oligonucleotide
tcgggacgtcgtacgacgtcccga

SEQ ID NO: 31 - DNA, 24 bp, Artificial Sequence
  Description of Artificial Sequence Synthetic oligonucleotide
tctgcacgtcgtacgacgtgcaga

SEQ ID NO: 32 - DNA, 24 bp, Artificial Sequence
  Description of Artificial Sequence Synthetic oligonucleotide
tcaggacgtcgtacgacgtcctga

SEQ ID NO: 33 - DNA, 24 bp, Artificial Sequence
  Description of Artificial Sequence Synthetic oligonucleotide
tcaaaactttgtacaaagttttga

SEQ ID NO: 34 - DNA, 24 bp, Artificial Sequence
  Description of Artificial Sequence Synthetic oligonucleotide
tcaaaaccctgtacagggttttga

SEQ ID NO: 35 - DNA, 22 bp, Artificial Sequence
  Description of Artificial Sequence Synthetic oligonucleotide
ctgccttttgtacaggatccta

SEQ ID NO: 36 - DNA, 22 bp, Artificial Sequence
  Description of Artificial Sequence Synthetic oligonucleotide
ctgccttttgtacaaaaggcag

SEQ ID NO: 37 - DNA, 22 bp, Artificial Sequence
  Description of Artificial Sequence Synthetic oligonucleotide
taggatcctgtacaggatccta

SEQ ID NO: 38 - DNA, 24 bp, Artificial Sequence
  Description of Artificial Sequence Synthetic oligonucleotide
tcaaaacgtcgtgagacagtttgg

SEQ ID NO: 39 - DNA, 49 bp, Artificial Sequence
  Description of Artificial Sequence Synthetic oligonucleotide
ggcatacaagtttcnnnacgtcgtacgacgtnnngacaatcgtctgtca

SEQ ID NO: 40 - DNA, 26 bp, Artificial Sequence
  Description of Artificial Sequence Synthetic primer
gcaactttagtgctgacacatacagg

SEQ ID NO: 41 - DNA, 24 bp, Artificial Sequence
  Description of Artificial Sequence Synthetic primer
acaaccttgattgcagacttgacc

SEQ ID NO: 42 - DNA, 50 bp, Artificial Sequence
  Description of Artificial Sequence Synthetic oligonucleotide
tggcatacaagtttctgccttttgtacaaaaggcagacaatcgtctgtca

SEQ ID NO: 43 - DNA, 15 bp, Artificial Sequence
  Description of Artificial Sequence Synthetic primer
ctannnttgaccttt

SEQ ID NO: 44 - DNA, 15 bp, Artificial Sequence
  Description of Artificial Sequence Synthetic primer
aaaggtcaannntag

SEQ ID NO: 45 - DNA, 22 bp, Artificial Sequence
  Description of Artificial Sequence Synthetic oligonucleotide
ctgcctgcctcggtgcgggcga

SEQ ID NO: 46 - DNA, 22 bp, Artificial Sequence
  Description of Artificial Sequence Synthetic oligonucleotide
cagactggctcgtgcaagccga

SEQ ID NO: 47 - DNA, 22 bp, Artificial Sequence
  Description of Artificial Sequence Synthetic oligonucleotide
tttacttacttttgtaggcatg

SEQ ID NO: 48 - DNA, 22 bp, Artificial Sequence
  Description of Artificial Sequence Synthetic oligonucleotide
taggacctgttatggaatttga

SEQ ID NO: 49 - DNA, 22 bp, Artificial Sequence
  Description of Artificial Sequence Synthetic oligonucleotide
taatctgttttcagagatgctg

SEQ ID NO: 50 - DNA, 22 bp, Artificial Sequence
  Description of Artificial Sequence Synthetic oligonucleotide
tgaaactctacttaaagttaca

SEQ ID NO: 51 - DNA, 22 bp, Artificial Sequence
  Description of Artificial Sequence Synthetic oligonucleotide
taagctcaaacctcacagtacg

SEQ ID NO: 52 - DNA, 22 bp, Artificial Sequence
  Description of Artificial Sequence Synthetic oligonucleotide
tacgacatttctgaaaagcatg

SEQ ID NO: 53 - DNA, 22 bp, Artificial Sequence
  Description of Artificial Sequence Synthetic oligonucleotide
cagaattgcggcgagcagtaag

SEQ ID NO: 54 - DNA, 22 bp, Artificial Sequence
  Description of Artificial Sequence Synthetic oligonucleotide
tgacctgttttatagaatttta

SEQ ID NO: 55 - DNA, 22 bp, Artificial Sequence
  Description of Artificial Sequence Synthetic oligonucleotide
taatacttcagtaataattatg

SEQ ID NO: 56 - DNA, 22 bp, Artificial Sequence
  Description of Artificial Sequence Synthetic oligonucleotide
caccactgtaccccaggttcta

SEQ ID NO: 57 - DNA, 22 bp, Artificial Sequence
  Description of Artificial Sequence Synthetic oligonucleotide
tgtaccccaggttctagtcatg

SEQ ID NO: 58 - DNA, 22 bp, Artificial Sequence
  Description of Artificial Sequence Synthetic oligonucleotide
cgggcctgtgggagcggggtca

SEQ ID NO: 59 - DNA, 22 bp, Artificial Sequence
  Description of Artificial Sequence Synthetic oligonucleotide
tggtatcttgcagacaagaaga

SEQ ID NO: 60 - DNA, 22 bp, Artificial Sequence
  Description of Artificial Sequence Synthetic oligonucleotide
ccaacccatgtgcatgagtaca

SEQ ID NO: 61 - DNA, 22 bp, Artificial Sequence
  Description of Artificial Sequence Synthetic oligonucleotide
caacccatgtgcatgagtacaa

SEQ ID NO: 62 - DNA, 22 bp, Artificial Sequence
  Description of Artificial Sequence Synthetic oligonucleotide
tggaattatgcagtttattaag

SEQ ID NO: 63 - DNA, 22 bp, Artificial Sequence
  Description of Artificial Sequence Synthetic oligonucleotide
tgttctcagaaagcagaggcca

SEQ ID NO: 64 - DNA, 22 bp, Artificial Sequence
  Description of Artificial Sequence Synthetic oligonucleotide
ttaaatcatggccggcagctca

SEQ ID NO: 65 - DNA, 22 bp, Artificial Sequence
  Description of Artificial Sequence Synthetic oligonucleotide
tttgcctgctgattagatttca

SEQ ID NO: 66 - DNA, 22 bp, Artificial Sequence
  Description of Artificial Sequence Synthetic oligonucleotide
ttggacctcttagaagatgtaa

SEQ ID NO: 67 - DNA, 22 bp, Artificial Sequence
  Description of Artificial Sequence Synthetic oligonucleotide
tatgacttccggaatgattctg

SEQ ID NO: 68 - DNA, 22 bp, Artificial Sequence
  Description of Artificial Sequence Synthetic oligonucleotide
ttccctgcggtaagtggtacca

SEQ ID NO: 69 - DNA, 22 bp, Artificial Sequence
  Description of Artificial Sequence Synthetic oligonucleotide
ccataccaggtaagcaggctgg

SEQ ID NO: 70 - DNA, 22 bp, Artificial Sequence
  Description of Artificial Sequence Synthetic oligonucleotide
cgaccctcgtccgcgaagatga

SEQ ID NO: 71 - DNA, 22 bp, Artificial Sequence
  Description of Artificial Sequence Synthetic oligonucleotide
ttaaattttctgattgggccta

SEQ ID NO: 72 - DNA, 22 bp, Artificial Sequence
  Description of Artificial Sequence Synthetic oligonucleotide
tgtgctgaggtagctgggcctg

SEQ ID NO: 73 - DNA, 22 bp, Artificial Sequence
  Description of Artificial Sequence Synthetic oligonucleotide
tcttactgtattacaggtctgg

SEQ ID NO: 74 - DNA, 22 bp, Artificial Sequence
  Description of Artificial Sequence Synthetic oligonucleotide
caaaaccaagaaacgaatcttg

SEQ ID NO: 75 - DNA, 22 bp, Artificial Sequence
  Description of Artificial Sequence Synthetic oligonucleotide
tttgccctaaaggaatatgcca

SEQ ID NO: 76 - DNA, 22 bp, Artificial Sequence
  Description of Artificial Sequence Synthetic oligonucleotide
cggacctacgtctcagggggaa

SEQ ID NO: 77 - DNA, 22 bp, Artificial Sequence
  Description of Artificial Sequence Synthetic oligonucleotide
ccatcttcatatccaaggtttg

SEQ ID NO: 78 - DNA, 22 bp, Artificial Sequence
  Description of Artificial Sequence Synthetic oligonucleotide
taaaccactttagttaggaaga

SEQ ID NO: 79 - DNA, 22 bp, Artificial Sequence
  Description of Artificial Sequence Synthetic oligonucleotide
cttactggctatttttatattg

SEQ ID NO: 80 - DNA, 22 bp, Artificial Sequence
  Description of Artificial Sequence Synthetic oligonucleotide
ctgcatgaatctctgagggcag

SEQ ID NO: 81 - DNA, 22 bp, Artificial Sequence
  Description of Artificial Sequence Synthetic oligonucleotide
tggtctctggaatgctagggag

SEQ ID NO: 82 - DNA, 22 bp, Artificial Sequence
  Description of Artificial Sequence Synthetic oligonucleotide
tagtcctgcctggatgggccca

SEQ ID NO: 83 - DNA, 22 bp, Artificial Sequence
  Description of Artificial Sequence Synthetic oligonucleotide
tgggatttttttggaaatgttg

SEQ ID NO: 84 - DNA, 22 bp, Artificial Sequence
  Description of Artificial Sequence Synthetic oligonucleotide
ccttccctccagcgttggccaa

SEQ ID NO: 85 - DNA, 22 bp, Artificial Sequence
  Description of Artificial Sequence Synthetic oligonucleotide
ttggctgtgccttcataggtca

SEQ ID NO: 86 - DNA, 22 bp, Artificial Sequence
  Description of Artificial Sequence Synthetic oligonucleotide
tgtgccttcataggtcatctag

SEQ ID NO: 87 - DNA, 22 bp, Artificial Sequence
  Description of Artificial Sequence Synthetic oligonucleotide
tcgaccccgctgcacagtccgg

SEQ ID NO: 88 - DNA, 22 bp, Artificial Sequence
  Description of Artificial Sequence Synthetic oligonucleotide
ctatatatcagctactgtgcca

SEQ ID NO: 89 - DNA, 22 bp, Artificial Sequence
  Description of Artificial Sequence Synthetic oligonucleotide
tggtccccaacatgcagggtca

SEQ ID NO: 90 - DNA, 22 bp, Artificial Sequence
  Description of Artificial Sequence Synthetic oligonucleotide
cccaacatgcagggtcatggag

SEQ ID NO: 91 - DNA, 22 bp, Artificial Sequence
  Description of Artificial Sequence Synthetic oligonucleotide
ctgaacccgtaaaggcagacaa

SEQ ID NO: 92 - DNA, 22 bp, Artificial Sequence
  Description of Artificial Sequence Synthetic oligonucleotide
cctgccccccaactttggagta

SEQ ID NO: 93 - DNA, 22 bp, Artificial Sequence
  Description of Artificial Sequence Synthetic oligonucleotide
ccttctccttgcccttagccca

SEQ ID NO: 94 - DNA, 22 bp, Artificial Sequence
  Description of Artificial Sequence Synthetic oligonucleotide
caggacaggtagcctggggcag

SEQ ID NO: 95 - DNA, 22 bp, Artificial Sequence
  Description of Artificial Sequence Synthetic oligonucleotide
tgacctgaaggccctggggcgg

SEQ ID NO: 96 - DNA, 22 bp, Artificial Sequence
  Description of Artificial Sequence Synthetic oligonucleotide
cgatactcagtgaggaggctgg

SEQ ID NO: 97 - DNA, 22 bp, Artificial Sequence
  Description of Artificial Sequence Synthetic oligonucleotide
ctccccggccccccagatcctg

SEQ ID NO: 98 - DNA, 22 bp, Artificial Sequence
  Description of Artificial Sequence Synthetic oligonucleotide
cacaacattggtgaggggggcg

SEQ ID NO: 99 - DNA, 22 bp, Artificial Sequence
  Description of Artificial Sequence Synthetic oligonucleotide
tgggaccctggcccctgtctga

SEQ ID NO: 100 - DNA, 22 bp, Artificial Sequence
  Description of Artificial Sequence Synthetic oligonucleotide
cgggacgagtaccggcgtctgg

SEQ ID NO: 101 - DNA, 22 bp, Artificial Sequence
  Description of Artificial Sequence Synthetic oligonucleotide
cgtgctgcccgacgaagtgctg

SEQ ID NO: 102 - DNA, 22 bp, Artificial Sequence
  Description of Artificial Sequence Synthetic oligonucleotide
ctggctccatccgcacggccga

SEQ ID NO: 103 - DNA, 22 bp, Artificial Sequence
  Description of Artificial Sequence Synthetic oligonucleotide
ctccccgccagattctgtgctg

SEQ ID NO: 104 - DNA, 22 bp, Artificial Sequence
  Description of Artificial Sequence Synthetic oligonucleotide
cagcacctacgccaaaggtaag

SEQ ID NO: 105 - DNA, 22 bp, Artificial Sequence
  Description of Artificial Sequence Synthetic oligonucleotide
tactcccaggcagtacggggtg

SEQ ID NO: 106 - DNA, 22 bp, Artificial Sequence
  Description of Artificial Sequence Synthetic oligonucleotide
tgtcatcatcacatctggggta

SEQ ID NO: 107 - DNA, 22 bp, Artificial Sequence
  Description of Artificial Sequence Synthetic oligonucleotide
tgagatccctcccactgtcccg

SEQ ID NO: 108 - DNA, 22 bp, Artificial Sequence
  Description of Artificial Sequence Synthetic oligonucleotide
cagcctgggcaacatggtgaca

SEQ ID NO: 109 - DNA, 22 bp, Artificial Sequence
  Description of Artificial Sequence Synthetic oligonucleotide
tggtatgctgccagtggggctg

SEQ ID NO: 110 - DNA, 22 bp, Artificial Sequence
  Description of Artificial Sequence Synthetic oligonucleotide
cctgacaggcagcctcagtggg

SEQ ID NO: 111 - DNA, 22 bp, Artificial Sequence
  Description of Artificial Sequence Synthetic oligonucleotide
cagcatgtaggaatggggtgta

SEQ ID NO: 112 - DNA, 22 bp, Artificial Sequence
  Description of Artificial Sequence Synthetic oligonucleotide
caggctgagaatgcagatataa

SEQ ID NO: 113 - DNA, 22 bp, Artificial Sequence
  Description of Artificial Sequence Synthetic oligonucleotide
caccacatctcagatgagccag

SEQ ID NO: 114 - DNA, 22 bp, Artificial Sequence
  Description of Artificial Sequence Synthetic oligonucleotide
ccatcctgctgtcagtggcccg

SEQ ID NO: 115 - DNA, 22 bp, Artificial Sequence
  Description of Artificial Sequence Synthetic oligonucleotide
tggcccggggcaaagtgtccga

SEQ ID NO: 116 - DNA, 22 bp, Artificial Sequence
  Description of Artificial Sequence Synthetic oligonucleotide
ccaactcagacacagcatcctg

SEQ ID NO: 117 - DNA, 22 bp, Artificial Sequence
  Description of Artificial Sequence Synthetic oligonucleotide
cccactccccaccctcagcctg

SEQ ID NO: 118 - DNA, 22 bp, Artificial Sequence
  Description of Artificial Sequence Synthetic oligonucleotide
ctgtcctcctgccctcagcaaa

SEQ ID NO: 119 - DNA, 22 bp, Artificial Sequence
  Description of Artificial Sequence Synthetic oligonucleotide
ccaaattcattcaaacatcctg

SEQ ID NO: 120 - DNA, 22 bp, Artificial Sequence
  Description of Artificial Sequence Synthetic oligonucleotide
caagcctcgacatgtcgtacaa

SEQ ID NO: 121 - DNA, 22 bp, Artificial Sequence
  Description of Artificial Sequence Synthetic oligonucleotide
caggacactttacttcggccga

SEQ ID NO: 122 - DNA, 22 bp, Artificial Sequence
  Description of Artificial Sequence Synthetic oligonucleotide
taatattggtttccagggggag

SEQ ID NO: 123 - DNA, 22 bp, Artificial Sequence
  Description of Artificial Sequence Synthetic oligonucleotide
tggccttttcaaggttattcca

SEQ ID NO: 124 - DNA, 22 bp, Artificial Sequence
  Description of Artificial Sequence Synthetic oligonucleotide
ttgtctaccaggtactggatca

SEQ ID NO: 125 - DNA, 22 bp, Artificial Sequence
  Description of Artificial Sequence Synthetic oligonucleotide
caggaccctcaggggcggcacg

SEQ ID NO: 126 - DNA, 22 bp, Artificial Sequence
  Description of Artificial Sequence Synthetic oligonucleotide
ttccatggtgatcgcaggttgg

SEQ ID NO: 127 - DNA, 22 bp, Artificial Sequence
  Description of Artificial Sequence Synthetic oligonucleotide
cccacccataggaatagtcgaa

SEQ ID NO: 128 - DNA, 22 bp, Artificial Sequence
  Description of Artificial Sequence Synthetic oligonucleotide
tcttactctgtcgctcaggctg

SEQ ID NO: 129 - DNA, 22 bp, Artificial Sequence
  Description of Artificial Sequence Synthetic oligonucleotide
taaaatggctgggtttggtaaa

SEQ ID NO: 130 - DNA, 22 bp, Artificial Sequence
  Description of Artificial Sequence Synthetic oligonucleotide
ctgaattatggctgcagttggg

SEQ ID NO: 131 - DNA, 22 bp, Artificial Sequence
  Description of Artificial Sequence Synthetic oligonucleotide
ccagcttgtgaaggtgagaaga

SEQ ID NO: 132 - DNA, 22 bp, Artificial Sequence
  Description of Artificial Sequence Synthetic oligonucleotide
ccatctcttttgggtgggtctg

SEQ ID NO: 133 - DNA, 22 bp, Artificial Sequence
  Description of Artificial Sequence Synthetic oligonucleotide
tggacctggagaggcaggggca

SEQ ID NO: 134 - DNA, 22 bp, Artificial Sequence
  Description of Artificial Sequence Synthetic oligonucleotide
ttaaacatatctgaaagtataa

SEQ ID NO: 135 - DNA, 22 bp, Artificial Sequence
  Description of Artificial Sequence Synthetic oligonucleotide
tctgaccctaatcgtgagactg

SEQ ID NO: 136 - DNA, 22 bp, Artificial Sequence
  Description of Artificial Sequence Synthetic oligonucleotide
tcttctgtggcaacgtggctca

SEQ ID NO: 137 - DNA, 22 bp, Artificial Sequence
  Description of Artificial Sequence Synthetic oligonucleotide
ttccccaccagctgtaggtttg

SEQ ID NO: 138 - DNA, 22 bp, Artificial Sequence
  Description of Artificial Sequence Synthetic oligonucleotide
tgccatattcttctttgttcag

SEQ ID NO: 139 - DNA, 22 bp, Artificial Sequence
  Description of Artificial Sequence Synthetic oligonucleotide
tggcctctggacggacatctcg

SEQ ID NO: 140 - DNA, 22 bp, Artificial Sequence
  Description of Artificial Sequence Synthetic oligonucleotide
ttacacagaattcttagtccca

SEQ ID NO: 141 - DNA, 22 bp, Artificial Sequence
  Description of Artificial Sequence Synthetic oligonucleotide
tgtcattctctctgtaggtctg

SEQ ID NO: 142 - DNA, 22 bp, Artificial Sequence
  Description of Artificial Sequence Synthetic oligonucleotide
tgagatgggaagtatagtgcag

SEQ ID NO: 143 - DNA, 22 bp, Artificial Sequence
  Description of Artificial Sequence Synthetic oligonucleotide
ccagaccaatgaaataagagta

SEQ ID NO: 144 - DNA, 22 bp, Artificial Sequence
  Description of Artificial Sequence Synthetic oligonucleotide
tggcaccatcgatgagatccag

SEQ ID NO: 145 - DNA, 22 bp, Artificial Sequence
  Description of Artificial Sequence Synthetic oligonucleotide
tctgctaccaggaagtgtccca

SEQ ID NO: 146 - DNA, 22 bp, Artificial Sequence
  Description of Artificial Sequence Synthetic oligonucleotide
caggctctgtccagcagtgtaa

SEQ ID NO: 147 - DNA, 22 bp, Artificial Sequence
  Description of Artificial Sequence Synthetic oligonucleotide
ctccctaatccaggctgttctg

SEQ ID NO: 148 - DNA, 22 bp, Artificial Sequence
  Description of Artificial Sequence Synthetic oligonucleotide
tggtctttcagtattcggatgg

SEQ ID NO: 149 - DNA, 22 bp, Artificial Sequence
  Description of Artificial Sequence Synthetic oligonucleotide
cagtattcggatggtaagtggg

SEQ ID NO: 150 - DNA, 22 bp, Artificial Sequence
  Description of Artificial Sequence Synthetic oligonucleotide
tgtactctatggtggaatttaa

SEQ ID NO: 151 - DNA, 22 bp, Artificial Sequence
  Description of Artificial Sequence Synthetic oligonucleotide
tagcacggtgaggcctggacca

SEQ ID NO: 152 - DNA, 22 bp, Artificial Sequence
  Description of Artificial Sequence Synthetic oligonucleotide
ccagcccagtattaggggcaga

SEQ ID NO: 153 - DNA, 22 bp, Artificial Sequence
  Description of Artificial Sequence Synthetic oligonucleotide
ccaaatgggctgcgttggcaga

SEQ ID NO: 154 - DNA, 22 bp, Artificial Sequence
  Description of Artificial Sequence Synthetic oligonucleotide
tggtcttttccacctgggcgag

SEQ ID NO: 155 - DNA, 22 bp, Artificial Sequence
  Description of Artificial Sequence Synthetic oligonucleotide
ttccacccccacacaaggctcg

SEQ ID NO: 156 - DNA, 22 bp, Artificial Sequence
  Description of Artificial Sequence Synthetic oligonucleotide
caacctcctgctggacatgcag

SEQ ID NO: 157 - DNA, 22 bp, Artificial Sequence
  Description of Artificial Sequence Synthetic oligonucleotide
tcgactcaataaagtcatcaaa

SEQ ID NO: 158 - DNA, 22 bp, Artificial Sequence
  Description of Artificial Sequence Synthetic oligonucleotide
caagatgcaggaggtggtggca

SEQ ID NO: 159 - DNA, 22 bp, Artificial Sequence
  Description of Artificial Sequence Synthetic oligonucleotide
ccatcttctcattgcagtatga

SEQ ID NO: 160 - DNA, 22 bp, Artificial Sequence
  Description of Artificial Sequence Synthetic oligonucleotide
tatgacgatggcagcggtatga

SEQ ID NO: 161 - DNA, 22 bp, Artificial Sequence
  Description of Artificial Sequence Synthetic oligonucleotide
cgacctcatcaaggttgtggag

SEQ ID NO: 162 - DNA, 22 bp, Artificial Sequence
  Description of Artificial Sequence Synthetic oligonucleotide
ctaactcggatccattagccaa

SEQ ID NO: 163 - DNA, 22 bp, Artificial Sequence
  Description of Artificial Sequence Synthetic oligonucleotide
tcggatccattagccaagggca

SEQ ID NO: 164 - DNA, 22 bp, Artificial Sequence
  Description of Artificial Sequence Synthetic oligonucleotide
tgtcctctttttatttagattg

SEQ ID NO: 165 - DNA, 22 bp, Artificial Sequence
  Description of Artificial Sequence Synthetic oligonucleotide
ctgactgccaagccatgggtag

SEQ ID NO: 166 - DNA, 22 bp, Artificial Sequence
  Description of Artificial Sequence Synthetic oligonucleotide
ccaaataaagtagaatataaga

SEQ ID NO: 167 - DNA, 22 bp, Artificial Sequence
  Description of Artificial Sequence Synthetic oligonucleotide
tgacacagagaaggatggcagg

SEQ ID NO: 168 - DNA, 22 bp, Artificial Sequence
  Description of Artificial Sequence Synthetic oligonucleotide
ctgccctgtattaaatagccta

SEQ ID NO: 169 - DNA, 22 bp, Artificial Sequence
  Description of Artificial Sequence Synthetic oligonucleotide
tttgatactggtttttgtcatg

SEQ ID NO: 170 - DNA, 22 bp, Artificial Sequence
  Description of Artificial Sequence Synthetic oligonucleotide
ctgtatctgtggccaaggtaaa

SEQ ID NO: 171 - DNA, 22 bp, Artificial Sequence
  Description of Artificial Sequence Synthetic oligonucleotide
taacccatcgcttgaagtggaa

SEQ ID NO: 172 - DNA, 22 bp, Artificial Sequence
  Description of Artificial Sequence Synthetic oligonucleotide
caagactaaatccttagttcag

SEQ ID NO: 173 - DNA, 22 bp, Artificial Sequence
  Description of Artificial Sequence Synthetic oligonucleotide
ttgaataaagtgttaggtttta

SEQ ID NO: 174 - DNA, 22 bp, Artificial Sequence
  Description of Artificial Sequence Synthetic oligonucleotide
tttaacttttcgtattaggttg

SEQ ID NO: 175 - DNA, 22 bp, Artificial Sequence
  Description of Artificial Sequence Synthetic oligonucleotide
ttaacttttcgtattaggttgg

SEQ ID NO: 176 - DNA, 22 bp, Artificial Sequence
  Description of Artificial Sequence Synthetic oligonucleotide
tgtaatgtatgttgaaagtata

SEQ ID NO: 177 - DNA, 22 bp, Artificial Sequence
  Description of Artificial Sequence Synthetic oligonucleotide
tttgcttccaaaatctatcaaa

SEQ ID NO: 178 - DNA, 22 bp, Artificial Sequence
  Description of Artificial Sequence Synthetic oligonucleotide
ttagctctttaaaagtagttca

SEQ ID NO: 179 - DNA, 22 bp, Artificial Sequence
  Description of Artificial Sequence Synthetic oligonucleotide
tcatccatccgcttctgggttg

SEQ ID NO: 180 - DNA, 22 bp, Artificial Sequence
  Description of Artificial Sequence Synthetic oligonucleotide
ctaacctttgttcggcagcttg

SEQ ID NO: 181 - DNA, 22 bp, Artificial Sequence
  Description of Artificial Sequence Synthetic oligonucleotide
tttaatatccgttacgatgctg

SEQ ID NO: 182 - DNA, 22 bp, Artificial Sequence
  Description of Artificial Sequence Synthetic oligonucleotide
tagtcctgctcaggaaggatag

SEQ ID NO: 183 - DNA, 22 bp, Artificial Sequence
  Description of Artificial Sequence Synthetic oligonucleotide
cctgctcaggaaggatagggca

SEQ ID NO: 184 - DNA, 22 bp, Artificial Sequence
  Description of Artificial Sequence Synthetic oligonucleotide
ttgtccctgaagaaagagaagg

SEQ ID NO: 185 - DNA, 22 bp, Artificial Sequence
  Description of Artificial Sequence Synthetic oligonucleotide
cactccagaaatgtgcgtggag

SEQ ID NO: 186 - DNA, 22 bp, Artificial Sequence
  Description of Artificial Sequence Synthetic oligonucleotide
cagcactggccattacagcaga

SEQ ID NO: 187 - DNA, 22 bp, Artificial Sequence
  Description of Artificial Sequence Synthetic oligonucleotide
ctggccattacagcagattctg

SEQ ID NO: 188 - DNA, 22 bp, Artificial Sequence
  Description of Artificial Sequence Synthetic oligonucleotide
cagaattagcagccctgtcaca

SEQ ID NO: 189 - DNA, 22 bp, Artificial Sequence
  Description of Artificial Sequence Synthetic oligonucleotide
cgggcctgtgggagcggggtca

SEQ ID NO: 190 - DNA, 22 bp, Artificial Sequence
  Description of Artificial Sequence Synthetic oligonucleotide
tggtatcttgcagacaagaaga

SEQ ID NO: 191 - DNA, 22 bp, Artificial Sequence
  Description of Artificial Sequence Synthetic oligonucleotide
ccaacccatgtgcatgagtaca

SEQ ID NO: 192 - DNA, 22 bp, Artificial Sequence
  Description of Artificial Sequence Synthetic oligonucleotide
caacccatgtgcatgagtacaa

SEQ ID NO: 193 - DNA, 22 bp, Artificial Sequence
  Description of Artificial Sequence Synthetic oligonucleotide
tggaattatgcagtttattaag

SEQ ID NO: 194 - DNA, 22 bp, Artificial Sequence
  Description of Artificial Sequence Synthetic oligonucleotide
tgttctcagaaagcagaggcca

SEQ ID NO: 195 - DNA, 22 bp, Artificial Sequence
  Description of Artificial Sequence Synthetic oligonucleotide
ttaaatcatggccggcagctca

SEQ ID NO: 196 - DNA, 22 bp, Artificial Sequence
  Description of Artificial Sequence Synthetic oligonucleotide
tttgcctgctgattagatttca

SEQ ID NO: 197 - DNA, 22 bp, Artificial Sequence
  Description of Artificial Sequence Synthetic oligonucleotide
ttggacctcttagaagatgtaa

SEQ ID NO: 198 - DNA, 22 bp, Artificial Sequence
  Description of Artificial Sequence Synthetic oligonucleotide
tatgacttccggaatgattctg

SEQ ID NO: 199 - DNA, 22 bp, Artificial Sequence
  Description of Artificial Sequence Synthetic oligonucleotide
ttccctgcggtaagtggtacca

SEQ ID NO: 200 - DNA, 22 bp, Artificial Sequence
  Description of Artificial Sequence Synthetic oligonucleotide
ccataccaggtaagcaggctgg

SEQ ID NO: 201 - DNA, 22 bp, Artificial Sequence
  Description of Artificial Sequence Synthetic oligonucleotide
cgaccctcgtccgcgaagatga

SEQ ID NO: 202 - DNA, 22 bp, Artificial Sequence
  Description of Artificial Sequence Synthetic oligonucleotide
ttaaattttctgattgggccta

SEQ ID NO: 203 - DNA, 22 bp, Artificial Sequence
  Description of Artificial Sequence Synthetic oligonucleotide
tgtgctgaggtagctgggcctg

SEQ ID NO: 204 - DNA, 22 bp, Artificial Sequence
  Description of Artificial Sequence Synthetic oligonucleotide
tcttactgtattacaggtctgg

SEQ ID NO: 205 - DNA, 22 bp, Artificial Sequence
  Description of Artificial Sequence Synthetic oligonucleotide
caaaaccaagaaacgaatcttg

SEQ ID NO: 206 - DNA, 22 bp, Artificial Sequence
  Description of Artificial Sequence Synthetic oligonucleotide
tttgccctaaaggaatatgcca

SEQ ID NO: 207 - DNA, 22 bp, Artificial Sequence
  Description of Artificial Sequence Synthetic oligonucleotide
cggacctacgtctcagggggaa

SEQ ID NO: 208 - DNA, 22 bp, Artificial Sequence
  Description of Artificial Sequence Synthetic oligonucleotide
ccatcttcatatccaaggtttg

SEQ ID NO: 209 - DNA, 22 bp, Artificial Sequence
  Description of Artificial Sequence Synthetic oligonucleotide
taaaccactttagttaggaaga

SEQ ID NO: 210 - DNA, 22 bp, Artificial Sequence
  Description of Artificial Sequence Synthetic oligonucleotide
cttactggctatttttatattg

SEQ ID NO: 211 - DNA, 22 bp, Artificial Sequence
  Description of Artificial Sequence Synthetic oligonucleotide
ctgcatgaatctctgagggcag

SEQ ID NO: 212 - DNA, 22 bp, Artificial Sequence
  Description of Artificial Sequence Synthetic oligonucleotide
tggtctctggaatgctagggag

SEQ ID NO: 213 - DNA, 22 bp, Artificial Sequence
  Description of Artificial Sequence Synthetic oligonucleotide
tagtcctgcctggatgggccca

SEQ ID NO: 214 - DNA, 22 bp, Artificial Sequence
  Description of Artificial Sequence Synthetic oligonucleotide
tgggatttttttggaaatgttg

SEQ ID NO: 215 - DNA, 22 bp, Artificial Sequence
  Description of Artificial Sequence Synthetic oligonucleotide
ccttccctccagcgttggccaa

SEQ ID NO: 216 - DNA, 22 bp, Artificial Sequence
  Description of Artificial Sequence Synthetic oligonucleotide
ttggctgtgccttcataggtca

SEQ ID NO: 217 - protein, 163 aa, Chlamydomonas reinhardtii
MNTKYNKEFLLYLAGFVDGDGSIIAQIKPNQSYKFKHQLSLAFQVTQKTQRRWFLDKLVDEIGVGYVRDRGSVSDYILSEIKPLHNFLTQLQPFLKLKQKQANLVLKIIWRLPSAKESPDKFLEVCTWVDQIAALNDSKTRKTTSETVRAVLDSLSEKKKSSP

SEQ ID NO: 218 - protein, 167 aa, Artificial Sequence
  Description of Artificial Sequence Synthetic polypeptide
MANTKYNKEFLLYLAGFVDGDGSIIAQIKPNQSYKFKHQLSLTFQVTQKTQRRWFLDKLVDEIGVGYVRDRGSVSNYILSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 219 - DNA, 22 bp, Artificial Sequence
  Description of Artificial Sequence Synthetic oligonucleotide
cgggctggggtacgtaggacag

SEQ ID NO: 220 - DNA, 22 bp, Artificial Sequence
  Description of Artificial Sequence Synthetic oligonucleotide
cgggctggggtaccccagcccg

SEQ ID NO: 221 - DNA, 22 bp, Artificial Sequence
  Description of Artificial Sequence Synthetic oligonucleotide
ctgtcctacgtacgtaggacag

SEQ ID NO: 222 - DNA, 22 bp, Artificial Sequence
  Description of Artificial Sequence Synthetic oligonucleotide
cgagatgtcgtacagaggtacg

SEQ ID NO: 223 - DNA, 22 bp, Artificial Sequence
  Description of Artificial Sequence Synthetic oligonucleotide
cgagatgtcgtacgacatctcg

SEQ ID NO: 224 - DNA, 22 bp, Artificial Sequence
  Description of Artificial Sequence Synthetic oligonucleotide
cgtacctctgtacagaggtacg

SEQ ID NO: 225 - DNA, 77 bp, Artificial Sequence
  Description of Artificial Sequence Synthetic primer
ggggacaagtttgtacaaaaaagcaggcttcgaaggagatagaaccatggccaataccaaatataacaaagagttcc

SEQ ID NO: 226 - DNA, 64 bp, Artificial Sequence
  Description of Artificial Sequence Synthetic primer
ggggaccactttgtacaagaaagctgggtttagtcggccgccggggaggatttcttcttctcgc

SEQ ID NO: 227 - DNA, 59 bp, Artificial Sequence
  Description of Artificial Sequence Synthetic primer
gcataaattactatacttctatagacacgcaaacacaaatacacagcggccttgccacc

SEQ ID NO: 228 - DNA, 48 bp, Artificial Sequence
  Description of Artificial Sequence Synthetic primer
ggctcgaggagctcgtctagaggatcgctcgagttatcagtcggccgc

SEQ ID NO: 229 - protein, 9 aa, Artificial Sequence
  Description of Artificial Sequence Synthetic peptide
LAGLIDADG

SEQ ID NO: 230 - DNA, 21 bp, Artificial Sequence
  Description of Artificial Sequence Synthetic oligonucleotide
ctgccttttgwcaaaaggcag

SEQ ID NO: 231 - DNA, 21 bp, Artificial Sequence
  Description of Artificial Sequence Synthetic oligonucleotide
ctgcctttttkaaggatccta

SEQ ID NO: 232 - DNA, 22 bp, Artificial Sequence
  Description of Artificial Sequence Synthetic oligonucleotide
taggatcctgwcaggatcctaa

SEQ ID NO: 233 - DNA, 21 bp, Artificial Sequence
  Description of Artificial Sequence Synthetic oligonucleotide
ctgccttttgwcaggatccta

SEQ ID NO: 234 - DNA, 11 bp, Artificial Sequence
  Description of Artificial Sequence Synthetic oligonucleotide
skghckkttkt